SEQUENCE LISTING (108 sequences)

SEQ ID NO: 1 - protein, 233 aa, synthetic construct
  source 1..233: note = anti-K11 LC
MGWSCIILFLVATATGVHSDIQMTQSPSSLSASVGDRVTITCRASQIVGTFVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 214 aa, synthetic construct
  source 1..214: note = anti-K11 mature LC
DIQMTQSPSSLSASVGDRVTITCRASQIVGTFVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 469 aa, synthetic construct
  source 1..469: note = anti-K11 knob HC
MGWSCIILFLVATATGAYAEVQLVESGGGLVQPGGSLRLSCAASGFTFSNSYISWVRQAPGKGLEWVAAINPAGGYTYYADSVKGRFTISADTSKNTAYLQMNSLRAEDTAVYYCAREWYFGGYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 450 aa, synthetic construct
  source 1..450: note = anti-K11 mature knob HC
EVQLVESGGGLVQPGGSLRLSCAASGFTFSNSYISWVRQAPGKGLEWVAAINPAGGYTYYADSVKGRFTISADTSKNTAYLQMNSLRAEDTAVYYCAREWYFGGYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 469 aa, synthetic construct
  source 1..469: note = anti-K11 hole HC
MGWSCIILFLVATATGAYAEVQLVESGGGLVQPGGSLRLSCAASGFTFSNSYISWVRQAPGKGLEWVAAINPAGGYTYYADSVKGRFTISADTSKNTAYLQMNSLRAEDTAVYYCAREWYFGGYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 450 aa, synthetic construct
  source 1..450: note = anti-K11 mature hole HC
EVQLVESGGGLVQPGGSLRLSCAASGFTFSNSYISWVRQAPGKGLEWVAAINPAGGYTYYADSVKGRFTISADTSKNTAYLQMNSLRAEDTAVYYCAREWYFGGYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 107 aa, synthetic construct
  source 1..107: note = anti-K11 LCVR
DIQMTQSPSSLSASVGDRVTITCRASQIVGTFVAWYQQKPGKAPKLLIYSASFLYSGVPSRFSGSGSGTDFTLTISSLQPEDFATYYCQQSYTTPPTFGQGTKVEIK

SEQ ID NO: 8 - protein, 120 aa, synthetic construct
  source 1..120: note = anti-K11 HCVR
EVQLVESGGGLVQPGGSLRLSCAASGFTFSNSYISWVRQAPGKGLEWVAAINPAGGYTYYADSVKGRFTISADTSKNTAYLQMNSLRAEDTAVYYCAREWYFGGYVMDYWGQGTLVTVSS

SEQ ID NO: 9 - protein, 11 aa, synthetic construct
  source 1..11: note = anti-K11 HVR-L1
RASQIVGTFVA

SEQ ID NO: 10 - protein, 7 aa, synthetic construct
  source 1..7: note = anti-K11 HVR-L2
SASFLYS

SEQ ID NO: 11 - protein, 9 aa, synthetic construct
  source 1..9: note = anti-K11 HVR-L3
QQSYTTPPT

SEQ ID NO: 12 - protein, 10 aa, synthetic construct
  source 1..10: note = anti-K11 HVR-H1
GFTFSNSYIS

SEQ ID NO: 13 - protein, 17 aa, synthetic construct
  source 1..17: note = anti-K11 HVR-H2
AINPAGGYTYYADSVKG

SEQ ID NO: 14 - protein, 13 aa, synthetic construct
  source 1..13: note = anti-K11 HVR-H3
AREWYFGGYVMDY

SEQ ID NO: 15 - protein, 234 aa, synthetic construct
  source 1..234: note = anti-K48 LC
MGWSCIILFLVATATGVHSDIQMTQSPSSLSASVGDRVTITCRASQSVSSAVAWYQQKPGKAPKLLIYSASSLYSGVPSRFSGSRSGTDFTLTISSLQPEDFATYYCQQSSYSSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 215 aa, synthetic construct
  source 1..215: note = anti-K48 mature LC
DIQMTQSPSSLSASVGDRVTITCRASQSVSSAVAWYQQKPGKAPKLLIYSASSLYSGVPSRFSGSRSGTDFTLTISSLQPEDFATYYCQQSSYSSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 468 aa, synthetic construct
  source 1..468: note = anti-K48 knob HC
MGWSCIILFLVATATGAYAEVQLVESGGGLVQPGGSLRLSCAASGFNISYSSMHWVRQAPGKGLEWVASIYSYYSYTSYADSVKGRFTISADTSKNTAYLQMNSLRAEDTAVYYCARSYSYHLGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 449 aa, synthetic construct
  source 1..449: note = anti-K48 mature knob HC
EVQLVESGGGLVQPGGSLRLSCAASGFNISYSSMHWVRQAPGKGLEWVASIYSYYSYTSYADSVKGRFTISADTSKNTAYLQMNSLRAEDTAVYYCARSYSYHLGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 468 aa, synthetic construct
  source 1..468: note = anti-K48 hole HC
MGWSCIILFLVATATGAYAEVQLVESGGGLVQPGGSLRLSCAASGFNISYSSMHWVRQAPGKGLEWVASIYSYYSYTSYADSVKGRFTISADTSKNTAYLQMNSLRAEDTAVYYCARSYSYHLGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 449 aa, synthetic construct
  source 1..449: note = anti-K48 mature hole HC
EVQLVESGGGLVQPGGSLRLSCAASGFNISYSSMHWVRQAPGKGLEWVASIYSYYSYTSYADSVKGRFTISADTSKNTAYLQMNSLRAEDTAVYYCARSYSYHLGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 108 aa, synthetic construct
  source 1..108: note = anti-K48 LCVR
DIQMTQSPSSLSASVGDRVTITCRASQSVSSAVAWYQQKPGKAPKLLIYSASSLYSGVPSRFSGSRSGTDFTLTISSLQPEDFATYYCQQSSYSSLITFGQGTKVEIK

SEQ ID NO: 22 - protein, 119 aa, synthetic construct
  source 1..119: note = anti-K48 HCVR
EVQLVESGGGLVQPGGSLRLSCAASGFNISYSSMHWVRQAPGKGLEWVASIYSYYSYTSYADSVKGRFTISADTSKNTAYLQMNSLRAEDTAVYYCARSYSYHLGMDYWGQGTLVTVSS

SEQ ID NO: 23 - protein, 11 aa, synthetic construct
  source 1..11: note = anti-K48 HVR-L1
RASQSVSSAVA

SEQ ID NO: 24 - protein, 7 aa, synthetic construct
  source 1..7: note = anti-K48 HVR-L2
SASSLYS

SEQ ID NO: 25 - protein, 10 aa, synthetic construct
  source 1..10: note = anti-K48 HVR-L3
QQSSYSSLIT

SEQ ID NO: 26 - protein, 10 aa, synthetic construct
  source 1..10: note = anti-K48 HVR-H1
GFNISYSSMH

SEQ ID NO: 27 - protein, 17 aa, synthetic construct
  source 1..17: note = anti-K48 HVR-H2
SIYSYYSYTSYADSVKG

SEQ ID NO: 28 - protein, 12 aa, synthetic construct
  source 1..12: note = anti-K48 HVR-H3
ARSYSYHLGMDY

SEQ ID NO: 29 - protein, 235 aa, synthetic construct
  source 1..235: note = anti-K63 LC
MGWSCIILFLVATATGVHSDIQMTQSPSSLSASVGDRVTITCRASQSVSSAVAWYQQKPGEAPKLLIYSARSLYSGVPSRFSGSRSGTDFTLTISSLQPEDFATYYCQQYSSYSSLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 216 aa, synthetic construct
  source 1..216: note = anti-K63 mature LC
DIQMTQSPSSLSASVGDRVTITCRASQSVSSAVAWYQQKPGEAPKLLIYSARSLYSGVPSRFSGSRSGTDFTLTISSLQPEDFATYYCQQYSSYSSLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 469 aa, synthetic construct
  source 1..469: note = anti-K63 knob HC
MGWSCIILFLVATATGAYAEVQLVESGGGLVQPGGSLRLSCAASGFNVKTGLIHWVRQAPGKGLEWVAYITPYYGSTSYADSVKGRFTISADTSKNTAYLQMNSLRAEDTAVYYCAREYYRWYTAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 450 aa, synthetic construct
  source 1..450: note = anti-K63 mature knob HC
EVQLVESGGGLVQPGGSLRLSCAASGFNVKTGLIHWVRQAPGKGLEWVAYITPYYGSTSYADSVKGRFTISADTSKNTAYLQMNSLRAEDTAVYYCAREYYRWYTAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 469 aa, synthetic construct
  source 1..469: note = anti-K63 hole HC
MGWSCIILFLVATATGAYAEVQLVESGGGLVQPGGSLRLSCAASGFNVKTGLIHWVRQAPGKGLEWVAYITPYYGSTSYADSVKGRFTISADTSKNTAYLQMNSLRAEDTAVYYCAREYYRWYTAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 450 aa, synthetic construct
  source 1..450: note = anti-K63 mature hole HC
EVQLVESGGGLVQPGGSLRLSCAASGFNVKTGLIHWVRQAPGKGLEWVAYITPYYGSTSYADSVKGRFTISADTSKNTAYLQMNSLRAEDTAVYYCAREYYRWYTAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 109 aa, synthetic construct
  source 1..109: note = anti-K63 LCVR
DIQMTQSPSSLSASVGDRVTITCRASQSVSSAVAWYQQKPGEAPKLLIYSARSLYSGVPSRFSGSRSGTDFTLTISSLQPEDFATYYCQQYSSYSSLFTFGQGTKVEIK

SEQ ID NO: 36 - protein, 120 aa, synthetic construct
  source 1..120: note = anti-K63 HCVR
EVQLVESGGGLVQPGGSLRLSCAASGFNVKTGLIHWVRQAPGKGLEWVAYITPYYGSTSYADSVKGRFTISADTSKNTAYLQMNSLRAEDTAVYYCAREYYRWYTAIDYWGQGTLVTVSS

SEQ ID NO: 37 - protein, 11 aa, synthetic construct
  source 1..11: note = anti-K63 HVR-L1
RASQSVSSAVA

SEQ ID NO: 38 - protein, 7 aa, synthetic construct
  source 1..7: note = anti-K63 HVR-L2
SARSLYS

SEQ ID NO: 39 - protein, 11 aa, synthetic construct
  source 1..11: note = anti-K63 HVR-L3
QQYSSYSSLFT

SEQ ID NO: 40 - protein, 10 aa, synthetic construct
  source 1..10: note = anti-K63 HVR-H1
GFNVKTGLIH

SEQ ID NO: 41 - protein, 17 aa, synthetic construct
  source 1..17: note = anti-K63 HVR-H2
YITPYYGSTSYADSVKG

SEQ ID NO: 42 - protein, 13 aa, synthetic construct
  source 1..13: note = anti-K63 HVR-H3
AREYYRWYTAIDY

SEQ ID NO: 43 - protein, 233 aa, synthetic construct
  source 1..233: note = anti-M1 LC
MGWSCIILFLVATATGVHSDIQMTQSPSSLSASVGDRVTITCRASQDVSTAVAWYQQKPGKAPKLLIYSAKFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 214 aa, synthetic construct
  source 1..214: note = anti-M1 mature LC
DIQMTQSPSSLSASVGDRVTITCRASQDVSTAVAWYQQKPGKAPKLLIYSAKFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 468 aa, synthetic construct
  source 1..468: note = anti-M1 knob HC
MGWSCIILFLVATATGAYAEVQLVESGGGLVQPGGSLRLSCAASGFTFSNTYISWVRQAPGKGLEWVASITPSSGQTDYADSVKGRFTISADTSKNTAYLQMNSLRAEDTAVYYCARTWLLRWVMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 449 aa, synthetic construct
  source 1..449: note = anti-M1 mature knob HC
EVQLVESGGGLVQPGGSLRLSCAASGFTFSNTYISWVRQAPGKGLEWVASITPSSGQTDYADSVKGRFTISADTSKNTAYLQMNSLRAEDTAVYYCARTWLLRWVMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 468 aa, synthetic construct
  source 1..468: note = anti-M1 hole HC
MGWSCIILFLVATATGAYAEVQLVESGGGLVQPGGSLRLSCAASGFTFSNTYISWVRQAPGKGLEWVASITPSSGQTDYADSVKGRFTISADTSKNTAYLQMNSLRAEDTAVYYCARTWLLRWVMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 449 aa, synthetic construct
  source 1..449: note = anti-M1 mature hole HC
EVQLVESGGGLVQPGGSLRLSCAASGFTFSNTYISWVRQAPGKGLEWVASITPSSGQTDYADSVKGRFTISADTSKNTAYLQMNSLRAEDTAVYYCARTWLLRWVMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 107 aa, synthetic construct
  source 1..107: note = anti-M1 LCVR
DIQMTQSPSSLSASVGDRVTITCRASQDVSTAVAWYQQKPGKAPKLLIYSAKFLYSGVPSRFSGSGSGTDFTLTISSLQPEDFATYYCQQSYTTPPTFGQGTKVEIK

SEQ ID NO: 50 - protein, 119 aa, synthetic construct
  source 1..119: note = anti-M1 HCVR
EVQLVESGGGLVQPGGSLRLSCAASGFTFSNTYISWVRQAPGKGLEWVASITPSSGQTDYADSVKGRFTISADTSKNTAYLQMNSLRAEDTAVYYCARTWLLRWVMDLWGQGTLVTVSS

SEQ ID NO: 51 - protein, 11 aa, synthetic construct
  source 1..11: note = anti-M1 HVR-L1
RASQDVSTAVA

SEQ ID NO: 52 - protein, 7 aa, synthetic construct
  source 1..7: note = anti-M1 HVR-L2
SAKFLYS

SEQ ID NO: 53 - protein, 9 aa, synthetic construct
  source 1..9: note = anti-M1 HVR-L3
QQSYTTPPT

SEQ ID NO: 54 - protein, 10 aa, synthetic construct
  source 1..10: note = anti-M1 HVR-H1
GFTFSNTYIS

SEQ ID NO: 55 - protein, 17 aa, synthetic construct
  source 1..17: note = anti-M1 HVR-H2
SITPSSGQTDYADSVKG

SEQ ID NO: 56 - protein, 12 aa, synthetic construct
  source 1..12: note = anti-M1 HVR-H3
ARTWLLRWVMDL

SEQ ID NO: 57 - protein, 238 aa, synthetic construct
  source 1..238: note = anti-RIP1 LC1
MGWSCIILFLVATATGVHSNIMMTQSPSSLAVSAGQKVTMNCKSSQNVIYTSNQRNYLAWYQQKPGQSPKLLIYWASTRVSGVPDRFTGSGSGTDFTLTISSVEVEDLAVYYCHQYLSSWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 58 - protein, 219 aa, synthetic construct
  source 1..219: note = anti-RIP1 mature LC1
NIMMTQSPSSLAVSAGQKVTMNCKSSQNVIYTSNQRNYLAWYQQKPGQSPKLLIYWASTRVSGVPDRFTGSGSGTDFTLTISSVEVEDLAVYYCHQYLSSWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 59 - protein, 238 aa, synthetic construct
  source 1..238: note = anti-RIP1 LC1 having one amino acid difference L104V
MGWSCIILFLVATATGVHSNIMMTQSPSSLAVSAGQKVTMNCKSSQNVIYTSNQRNYLAWYQQKPGQSPKLLIYWASTRVSGVPDRFTGSGSGTDFTLTISSVEVEDLAVYYCHQYLSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 219 aa, synthetic construct
  source 1..219: note = anti-RIP1 mature LC1 having one amino acid difference L104V
NIMMTQSPSSLAVSAGQKVTMNCKSSQNVIYTSNQRNYLAWYQQKPGQSPKLLIYWASTRVSGVPDRFTGSGSGTDFTLTISSVEVEDLAVYYCHQYLSSWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 238 aa, synthetic construct
  source 1..238: note = anti-RIP1 LC2
MGWSCIILFLVATATGVHSNIMMTQSPSSLAVSAGQKVTMNCKSSQNVIYTSNQRNYLAWYQQKPGQSPKLLIYGASNRYTGVPDRFTGSGSGTDFTLTISSVEVEDLAVYYCHQYLSSWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 62 - protein, 219 aa, synthetic construct
  source 1..219: note = anti-RIP1 mature LC2
NIMMTQSPSSLAVSAGQKVTMNCKSSQNVIYTSNQRNYLAWYQQKPGQSPKLLIYGASNRYTGVPDRFTGSGSGTDFTLTISSVEVEDLAVYYCHQYLSSWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 63 - protein, 467 aa, synthetic construct
  source 1..467: note = anti-RIP1 HC
MGWSCIILFLVATATGAYADVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTDNGKTELNYKNTEPVLDSDGSYFMYSKLRVEKKNWVERNSYSCSVVHEGLHNHHTTKSFSRTPG

SEQ ID NO: 64 - protein, 448 aa, synthetic construct
  source 1..448: note = anti-RIP1 mature HC
DVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTDNGKTELNYKNTEPVLDSDGSYFMYSKLRVEKKNWVERNSYSCSVVHEGLHNHHTTKSFSRTPG

SEQ ID NO: 65 - protein, 468 aa, synthetic construct
  source 1..468: note = anti-RIP1 HC having two amino acid differences in constant region
MGWSCIILFLVATATGAYADVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 66 - protein, 449 aa, synthetic construct
  source 1..449: note = anti-RIP1 mature HC having two amino acid differences in constant region
DVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTGPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 67 - protein, 468 aa, synthetic construct
  source 1..468: note = anti-RIP1 knob HC
MGWSCIILFLVATATGAYADVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 449 aa, synthetic construct
  source 1..449: note = anti-RIP1 mature knob HC
DVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 468 aa, synthetic construct
  source 1..468: note = anti-RIP1 hole HC
MGWSCIILFLVATATGAYADVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - protein, 449 aa, synthetic construct
  source 1..449: note = anti-RIP1 mature hole HC
DVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 71 - protein, 112 aa, synthetic construct
  source 1..112: note = anti-RIP1 LC1 LCVR having one amino acid difference L104V
NIMMTQSPSSLAVSAGQKVTMNCKSSQNVIYTSNQRNYLAWYQQKPGQSPKLLIYWASTRVSGVPDRFTGSGSGTDFTLTISSVEVEDLAVYYCHQYLSSWTFGGGTKVEIK

SEQ ID NO: 72 - protein, 112 aa, synthetic construct
  source 1..112: note = anti-RIP1 LC2 LCVR
NIMMTQSPSSLAVSAGQKVTMNCKSSQNVIYTSNQRNYLAWYQQKPGQSPKLLIYGASNRYTGVPDRFTGSGSGTDFTLTISSVEVEDLAVYYCHQYLSSWTFGGGTKLEIK

SEQ ID NO: 73 - protein, 119 aa, synthetic construct
  source 1..119: note = anti-RIP1 HCVR
DVQLVESGGGLVQPGGSRKLSCAASGFTFGSFGMHWVRQTPEKRLEWIAYISSGGSTIYYADTVKGRFTISRDTPKNTLFLQMTSLRSEDTAMYFCARSIMISTEFDYWGQGTTLTVSS

SEQ ID NO: 74 - protein, 17 aa, synthetic construct
  source 1..17: note = anti-RIP1 LC1 HVR-L1
KSSQNVIYTSNQRNYLA

SEQ ID NO: 75 - protein, 7 aa, synthetic construct
  source 1..7: note = anti-RIP1 LC1 HVR-L2
WASTRVS

SEQ ID NO: 76 - protein, 8 aa, synthetic construct
  source 1..8: note = anti-RIP1 LC1 HVR-L3
HQYLSSWT

SEQ ID NO: 77 - protein, 7 aa, synthetic construct
  source 1..7: note = anti-RIP1 LC2 HVR-L2
GASNRYT

SEQ ID NO: 78 - protein, 10 aa, synthetic construct
  source 1..10: note = anti-RIP1 HVR-H1
GFTFGSFGMH

SEQ ID NO: 79 - protein, 17 aa, synthetic construct
  source 1..17: note = anti-RIP1 HVR-H2
YISSGGSTIYYADTVKG

SEQ ID NO: 80 - protein, 12 aa, synthetic construct
  source 1..12: note = anti-RIP1 HVR-H3
ARSIMISTEFDY

SEQ ID NO: 81 - protein, 238 aa, synthetic construct
  source 1..238: note = anti-RIP2 LC
MGWSCIILFLVATATGVHSNIMMTQSPSSLAVSAGEKVTMNCKSSQSVLYSSNQRNYLAWYQQKPGQSPKLLIYWASIRESGVPDRFTGSGSGTDFTLTISSVQAEDLAVYYCHQYLSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 82 - protein, 219 aa, synthetic construct
  source 1..219: note = anti-RIP2 mature LC
NIMMTQSPSSLAVSAGEKVTMNCKSSQSVLYSSNQRNYLAWYQQKPGQSPKLLIYWASIRESGVPDRFTGSGSGTDFTLTISSVQAEDLAVYYCHQYLSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 83 - protein, 238 aa, synthetic construct
  source 1..238: note = anti-RIP2 LC having one amino acid difference L104V
MGWSCIILFLVATATGVHSNIMMTQSPSSLAVSAGEKVTMNCKSSQSVLYSSNQRNYLAWYQQKPGQSPKLLIYWASIRESGVPDRFTGSGSGTDFTLTISSVQAEDLAVYYCHQYLSSYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 219 aa, synthetic construct
  source 1..219: note = anti-RIP2 mature LC having one amino acid difference L104V
NIMMTQSPSSLAVSAGEKVTMNCKSSQSVLYSSNQRNYLAWYQQKPGQSPKLLIYWASIRESGVPDRFTGSGSGTDFTLTISSVQAEDLAVYYCHQYLSSYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 466 aa, synthetic construct
  source 1..466: note = anti-RIP2 HC
MGWSCIILFLVATATGAYAEVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 86 - protein, 447 aa, synthetic construct
  source 1..447: note = anti-RIP2 mature HC
EVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 87 - protein, 466 aa, synthetic construct
  source 1..466: note = anti-RIP2 HC having one amino acid difference A113S
MGWSCIILFLVATATGAYAEVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 88 - protein, 447 aa, synthetic construct
  source 1..447: note = anti-RIP2 mature HC having one amino acid difference A113S
EVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 89 - protein, 472 aa, synthetic construct
  source 1..472: note = anti-RIP2 knob HC having one amino acid difference A113S
MGWSCIILFLVATATGAYAEVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 453 aa, synthetic construct
  source 1..453: note = anti-RIP2 mature knob HC having one amino acid difference A113S
EVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 472 aa, synthetic construct
  source 1..472: note = anti-RIP2 hole HC having one amino acid difference A113S
MGWSCIILFLVATATGAYAEVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 453 aa, synthetic construct
  source 1..453: note = anti-RIP2 mature hole HC having one amino acid difference A113S
EVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 93 - protein, 112 aa, synthetic construct
  source 1..112: note = anti-RIP2 LCVR having one amino acid difference L104V
NIMMTQSPSSLAVSAGEKVTMNCKSSQSVLYSSNQRNYLAWYQQKPGQSPKLLIYWASIRESGVPDRFTGSGSGTDFTLTISSVQAEDLAVYYCHQYLSSYTFGGGTKVEIK

SEQ ID NO: 94 - protein, 123 aa, synthetic construct
  source 1..123: note = anti-RIP2 HCVR having one amino acid difference A113S
EVQLQQSGPELEKPGASVKISCKASGYSFTAYNMNWVKQSNGKSLEWIGNIDPFYGDLNYSQKFKGKATLTVDKSSSTAYMQLMSLTSEDSAVYYCVRSNYYASVYGAWFAHWGQGTLVTVSS

SEQ ID NO: 95 - protein, 17 aa, synthetic construct
  source 1..17: note = anti-RIP2 HVR-L1
KSSQSVLYSSNQRNYLA

SEQ ID NO: 96 - protein, 7 aa, synthetic construct
  source 1..7: note = anti-RIP2 HVR-L2
WASIRES

SEQ ID NO: 97 - protein, 8 aa, synthetic construct
  source 1..8: note = anti-RIP2 HVR-L3
HQYLSSYT

SEQ ID NO: 98 - protein, 10 aa, synthetic construct
  source 1..10: note = anti-RIP2 HVR-H1
GYSFTAYNMN

SEQ ID NO: 99 - protein, 17 aa, synthetic construct
  source 1..17: note = anti-RIP2 HVR-H2
NIDPFYGDLNYSQKFKG

SEQ ID NO: 100 - protein, 16 aa, synthetic construct
  source 1..16: note = anti-RIP2 HVR-H3
VRSNYYASVYGAWFAH

SEQ ID NO: 101 - protein, 540 aa, Homo sapiens
  source 1..540: note = RIP2 polypeptide sequence (UniProt O43353)
MNGEAICSALPTIPYHKLADLRYLSRGASGTVSSARHADWRVQVAVKHLHIHTPLLDSERKDVLREAEILHKARFSYILPILGICNEPEFLGIVTEYMPNGSLNELLHRKTEYPDVAWPLRFRILHEIALGVNYLHNMTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEEITFLEAVIQLKKTKLQSVSSAIHLCDKKKMELSLNIPVNHGPQEESCGSSQLHENSGSPETSRSLPAPQDNDFLSRKAQDCYFMKLHHCPGNHSWDSTISGSQRAAFCDHKTTPCSSAIINPLSTAGNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

SEQ ID NO: 102 - protein, 12 aa, Homo sapiens
  source 1..12: note = C-terminal residues 529-540 of RIP2 polypeptide
SPSLNLLQNKSM

SEQ ID NO: 103 - protein, 671 aa, Homo sapiens
  source 1..671: note = RIP1 polypeptide (UniProt Q13546)
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 104 - protein, 10 aa, Homo sapiens
  source 1..10: note = TRADD: 125-134
LDALLADEER

SEQ ID NO: 105 - protein, 9 aa, Homo sapiens
  source 1..9: note = TNFR1: 415-423
EATLELLGR

SEQ ID NO: 106 - protein, 10 aa, Homo sapiens
  source 1..10: note = RIP1: 154-163
IADLGLASFK

SEQ ID NO: 107 - protein, 16 aa, Homo sapiens
  source 1..16: note = NOD2: 107-118
LIAAAQEAQADSQSPK

SEQ ID NO: 108 - protein, 9 aa, Homo sapiens
  source 1..9: note = IRAK1: 343-351
SSNVLLDER

(19) United States
(12) Patent Application Publication
(10) Pub. No.: US 2024/0369575 A1
(43) Pub. Date: Nov. 7, 2024

(54) ANTI-POLYUBIQUITIN MULTISPECIFIC ANTIBODIES

(71) Applicant: Genentech, Inc., South San Francisco, CA (US)

(72) Inventors: Marissa Lynn MATSUMOTO, South San Francisco, CA (US); Domagoj VUCIC, South San Francisco, CA (US); Tatiana GONCHAROV, South San Francisco, CA (US)

(73) Assignee: Genentech, Inc., South San Francisco, CA (US)

(21) Appl. No.: 18/587,851

(22) Filed: Feb. 26, 2024

Related U.S. Application Data
(63) Continuation of application No. PCT/US2022/075595, filed on Aug. 29, 2022.
(60) Provisional application No. 63/247,776, filed on Sep. 23, 2021.
(60) Provisional application No. 63/238,749, filed on Aug. 30, 2021.

(51) Int. Cl. G01N 33/68; C07K 16/40; G01N 33/53

(52) U.S. Cl. CPC G01N 33/6893; C07K 16/40; G01N 33/53; C07K 2317/31; C07K 2317/56; G01N 2333/4704; G01N 2333/7151; G01N 2333/912; G01N 2800/065

(57) ABSTRACT

Multispecific antibodies comprising a first half antibody comprising a first antigen binding site that binds to a polyubiquitin; and a second half antibody comprising a second antigen binding site that binds a pro-inflammatory protein, such as receptor-interacting protein kinase 1 (RIP1) or receptor-interacting protein kinase 2 (RIP2), as well as methods for using the antibodies, are provided.

### CROSS-REFERENCE TO RELATED APPLICATIONS

This application is a continuation of International Application No. PCT/US2022/075595 having an international filing date of Aug. 29, 2022, which claims benefit of priority of U.S. Provisional Patent Application No. 63/238,749, filed Aug. 30, 2021 and U.S. Provisional Patent Application No. 63/247,776, filed Sep. 23, 2021, each of which is incorporated by herein by reference in their entirety.

### SEQUENCE LISTING

This application contains a Sequence Listing which has been submitted electronically in XML format and is hereby incorporated by reference in its entirety. Said XML copy, created on Feb. 3, 2024, is named P36650-US-2_Sequence_Listing.xml and is 135,622 bytes in size.

### FIELD OF THE INVENTION

The present invention relates to anti-polyubiquitin multispecific antibodies and methods of making and using the same.

### INTRODUCTION AND SUMMARY

Ubiquitin is a small protein that has important regulatory roles in a wide variety of cellular pathways. The best known of these is ubiquitin's role in protein degradation, where covalent attachment of ubiquitin to a target protein enables that targeted protein to be recognized and destroyed by the 26S proteasome (see Wilkinson, Semin. Cell Devel. Biol. 11(3): 141-148 (2000)). The covalent attachment of ubiquitin, a 76 amino acid protein, to a target protein is a three-step enzymatic process (Pickart, Annu. Rev. Biochem. 70: 503-533 (2001)). First, ubiquitin-activating enzyme E1 forms an ubiquitin-E1 thioester in an ATP-dependent reaction. The ubiquitin is transferred from the ubiquitin-E1 thioester to a member of the ubiquitin-conjugating enzyme (E2) family in the second step. In the third step, with the assistance of a ubiquitin-protein ligase (E3), an isopeptide bond is formed between the carboxyl terminus of ubiquitin and the ε-amino group of a lysine residue on the target protein. Enzymes termed deubiquitinases remove ubiquitin moieties from target proteins (Guterman and Glickman, Curr. Prot. Pep. Sci. 5: 201-210 (2004)).

Ubiquitin contains seven lysine residues (Lys6, Lys 11, Lys27, Lys33, Lys29, Lys48, and Lys63), and thus ubiquitin itself may serve as a target protein for ubiquitination (Peng et al., Nat. Biotechnol. 21: 921-926 (2003); Pickart and Fushman, Curr. Opin. Chem. Biol. 8:610-616 (2004)). The molecule produced upon ubiquitination of a ubiquitin protein is termed a polyubiquitin molecule and may comprise two or more ubiquitin moieties.

Additionally, linear polyubiquitin linkages (also referred to as M1 linkages) form in which the C-terminal glycine of ubiquitin is conjugated to the α-amino group of the N-terminal methionine of another ubiquitin molecule. Iwai and Tokunaga, EMBO Reports 10:706-713 (2009). Linear polyubiquitin is formed via the linear ubiquitin chain assembly complex (LUBAC) which is composed of two ring finger proteins, HOIL-1L and HOIP. Tokunaga et al., Nat. Cell Biol. 11:123-132 (2009). It is believed that genetically encoded, unanchored linear polyubiquitin does not exist in cells as its C-terminus is vulnerable to cleavage by isopeptidase T. Iwai and Tokunaga, EMBO Reports 10:706-713 (2009). This observation suggests that linear polyubiquitin is assembled onto a substrate protein post-translationally and that conjugated linear polyubiquitin molecules are potential modulators of protein activity and function. Id. For example, linear polyubiquitination of the NF-κB essential modulator (NEMO) has been shown to play a role in NF-κB activation. Id. Different ubiquitin chains can transmit specific and distinct biochemical and biological messages leading to activation or abrogation of cellular signaling, or modulation of protein stability (Ikeda, F., et al., Cell 143:677-681 (2010); Rape, M., Nat Rev Mol Cell Biol 19:59-70 (2018)).

Receptor-interacting protein-1 kinase (“RIP1” or “RIPK1”) is a serine/threonine protein kinase. RIP1 is a regulator of cell signaling that is involved, among other things, in the mediation of programmed cell death pathways, e.g., necroptosis. The best studied form of necroptotic cell death is initiated by TNFa (tumor necrosis factor), but necroptosis can also be induced by other members of the TNFa death ligand family (Fas and TRAIL/Apo2L), interferons, Toll-like receptors (TLRs) signaling and viral infection via the DNA sensor DAI (DNA-dependent activator of interferon regulatory factor). Van den Berghe et al. (2014) Nature Reviews. Molecular cell biology 15:135-147 (2014); Newton, K. Trends in Cell Biology 25:347-353 (2015); de Almagro, M. C. and Vucic, D. Semin Cell Dev Biol. 39:56-62 (2015). Binding of TNFa to the TNFRl (TNF receptor 1) prompts TNFRl trimerization and formation of an intracellular complex, Complex-I. TRADD (TNF receptor associated death domain protein) binds to the intracellular death domain of TNFRl and recruits the protein kinase RIP1 (receptor-interacting protein 1) through the death domain present in both proteins. Chen, Z. J. Immunological reviews 246:95-106 (2012).

Receptor-interacting serine/threonine-protein kinase 2 (“RIP2” or “RIPK2”) is a serine/threonine/tyrosine kinase and plays an essential role in modulation of adaptive and innate immune responses. Once recruited through CARD-CARD domains by activated NOD1 and NOD2, RIPK2 autophosphorylates and undergoes K63-linked polyubiquitination by the ubiquitin ligases XIAP, BIRC2 and BIRC3. The polyubiquitinated protein induces K63-linked polyubiquitination of IKBKG/NEMO and subsequent activation of IKBKB/IKKB. Subsequently, NF-kappa-B is released and translocates into the nucleus where it activates the transcription of hundreds of genes involved in immune response, growth control, or protection against apoptosis.

It would be beneficial to provide compositions and methods that can recognize and distinguish between modifications of RIP1 and RIP2 proteins, and to provide compositions and methods that are effective in targeting and modulating polyubiquitin-mediated pathways. The present disclosure aims to meet one or more of these needs or provide other benefits.

The following non-limiting embodiments are provided.
Embodiment 1. A method of determining the presence of a polyubiquitinated protein in a sample suspected of containing a polyubiquitinated protein, wherein the polyubiquitinated protein is a pro-inflammatory protein and comprises a polyubiquitin, comprising exposing the sample to at least one multispecific antibody comprising a first half antibody comprising a first antigen binding site that binds to a polyubiquitin; and a second half antibody comprising a second antigen binding site that binds the pro-inflammatory protein, and
   determining the binding of the at least one antibody to a polyubiquitinated protein in the sample.
Embodiment 2. The method of Embodiment 1, wherein the polyubiquitinated protein comprises a M1-linked polyubiquitin and/or a K63-linked polyubiquitin.
Embodiment 3. The method of any one of the preceding Embodiments, wherein the pro-inflammatory protein is a component of one or more signaling complexes.
Embodiment 4. The method of any one of the preceding Embodiments, wherein the pro-inflammatory protein is receptor-interacting protein kinase 1 (RIP1), receptor-interacting protein kinase 2 (RIP2), cellular inhibitors of apoptosis 1 and 2 (c-IAP1/2), Tumor Necrosis Factor Receptor 1 (TNFR1), linear ubiquitin chain assembly complex (LUBAC), and/or nuclear factor-kappa B (NF-κB) essential modulator (NEMO).
Embodiment 5. The method of any one of the preceding Embodiments, wherein the pro-inflammatory protein is RIP1.
Embodiment 6. The method of any one of Embodiments 1-4, wherein the pro-inflammatory protein is RIP2.
Embodiment 7. The method of any one of the preceding Embodiments, wherein the pro-inflammatory protein has an elevated level of ubiquitination in the inflammatory state relative to the level of ubiquitination when not in the inflammatory state.
Embodiment 8. The method of any one of the preceding Embodiments, wherein the pro-inflammatory protein has a level of ubiquitination of at least 1-fold, 2-fold, 3-fold, 4-fold, 5-fold, 6-fold, 7-fold, 8-fold, 9-fold, 10-fold, 11-fold, 12-fold, 1-fold to 12-fold, 2-fold to 12-fold, 3-fold to 12-fold, 4-fold to 12-fold, 5-fold to 12-fold, 6-fold to 12-fold, 7-fold to 12-fold, 8-fold to 12-fold, 9-fold to 12-fold, 10-fold to 12-fold, or 11-fold to 12-fold in the inflammatory state relative to the level of ubiquitination when not in the inflammatory state.
Embodiment 9. The method of any one of the preceding Embodiments, wherein an elevated level of ubiquitination correlates to an increase in severity of an inflammatory disease state.
Embodiment 10. The method of any one of the preceding Embodiments, wherein the pro-inflammatory protein is associated with an inflammatory disease, such as inflammatory bowel disease, Crohn's disease, diverticulitis, and ulcerative colitis.
Embodiment 11. The method of any one of the preceding Embodiments, wherein the pro-inflammatory protein is associated with Crohn's disease.
Embodiment 12. The method of any one of the preceding Embodiments, wherein the pro-inflammatory protein is associated with ulcerative colitis.
Embodiment 13. A multispecific antibody comprising a first half antibody comprising a first antigen binding site that binds to a polyubiquitin; and a second half antibody comprising a second antigen binding site that binds receptor-interacting protein kinase 1 (RIP1).
Embodiment 14. The antibody of Embodiment 13, which selectively recognizes polyubiquitinated RIP1.
Embodiment 15. The antibody of Embodiment 13 or 14, which does not recognize receptor-interacting protein kinase 2 (RIP2) and/or does not recognize non-ubiquitinated RIP1.
Embodiment 16. A multispecific antibody comprising a first half antibody comprising a first antigen binding site that binds to a polyubiquitin; and a second half antibody comprising a second antigen binding site that binds receptor-interacting protein kinase 2 (RIP2).
Embodiment 17. The antibody of Embodiment 16, which selectively recognizes polyubiquitinated RIP2.
Embodiment 18. The antibody of Embodiment 16 or 17, which does not recognize RIP1, XIAP, and/or c-IAP1, and/or does not recognize non-ubiquitinated RIP2.
Embodiment 19. The antibody of any one of Embodiments 13 to 18, wherein the polyubiquitin has homogeneous topology.
Embodiment 20. The antibody of any one of Embodiments 13 to 19, wherein the polyubiquitin comprises a K11 linkage.
Embodiment 21. The antibody of any one of Embodiments 13 to 20, wherein the polyubiquitin comprises a K48 linkage.
Embodiment 22. The antibody of any one of Embodiments 13 to 21, wherein the polyubiquitin comprises a K63 linkage.
Embodiment 23. The antibody of any one of Embodiments 13 to 22, wherein the polyubiquitin comprises a M1 linkage.
Embodiment 24. The antibody of any one of Embodiments 13 to 23, comprising a first half antibody that comprises:
   a. (i) HVR-L1 comprising the amino acid sequence of SEQ ID NO: 9,
      (ii) HVR-L2 comprising the amino acid sequence of SEQ ID NO: 10,
      (iii) HVR-L3 comprising the amino acid sequence of SEQ ID NO: 11,
      (iv) HVR-H1 comprising the amino acid sequence of SEQ ID NO: 12,
      (v) HVR-H2 comprising the amino acid sequence of SEQ ID NO: 13, and
      (vi) HVR-H3 comprising the amino acid sequence of SEQ ID NO: 14;
   b. (i) HVR-L1 comprising the amino acid sequence of SEQ ID NO: 23,
      (ii) HVR-L2 comprising the amino acid sequence of SEQ ID NO: 24,
      (iii) HVR-L3 comprising the amino acid sequence of SEQ ID NO: 25,
      (iv) HVR-H1 comprising the amino acid sequence of SEQ ID NO: 26,
      (v) HVR-H2 comprising the amino acid sequence of SEQ ID NO: 27, and
      (vi) HVR-H3 comprising the amino acid sequence of SEQ ID NO: 28;
   c. (i) HVR-L1 comprising the amino acid sequence of SEQ ID NO: 37,
      (ii) HVR-L2 comprising the amino acid sequence of SEQ ID NO: 38,
      (iii) HVR-L3 comprising the amino acid sequence of SEQ ID NO: 39,
      (iv) HVR-H1 comprising the amino acid sequence of SEQ ID NO: 40,
      (v) HVR-H2 comprising the amino acid sequence of SEQ ID NO: 41, and
      (vi) HVR-H3 comprising the amino acid sequence of SEQ ID NO: 42; or
   d. (i) HVR-L1 comprising the amino acid sequence of SEQ ID NO: 51,
      (ii) HVR-L2 comprising the amino acid sequence of SEQ ID NO: 52,
      (iii) HVR-L3 comprising the amino acid sequence of SEQ ID NO: 53,
      (iv) HVR-H1 comprising the amino acid sequence of SEQ ID NO: 54,
      (v) HVR-H2 comprising the amino acid sequence of SEQ ID NO: 55, and
      (vi) HVR-H3 comprising the amino acid sequence of SEQ ID NO: 56.
Embodiment 25. The antibody of any one of Embodiments 13 to 15 or 19 to 24, comprising a second half antibody that comprises:
   a. (i) HVR-L1 comprising the amino acid sequence of SEQ ID NO: 74,
      (ii) HVR-L2 comprising the amino acid sequence of SEQ ID NO: 75,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80; or
   b. (i) HVR-L1 comprising the amino acid sequence of SEQ ID NO: 74,
      (ii) HVR-L2 comprising the amino acid sequence of SEQ ID NO: 77,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80.
Embodiment 26. The antibody of any one of Embodiments 16 to 24, comprising a second half antibody that comprises:
   (i) HVR-L1 comprising the amino acid sequence of SEQ ID NO: 95,
      (ii) HVR-L2 comprising the amino acid sequence of SEQ ID NO: 96,
      (iii) HVR-L3 comprising the amino acid sequence of SEQ ID NO: 97,
      (iv) HVR-H1 comprising the amino acid sequence of SEQ ID NO: 98,
      (v) HVR-H2 comprising the amino acid sequence of SEQ ID NO: 99, and
      (vi) HVR-H3 comprising the amino acid sequence of SEQ ID NO: 100.
Embodiment 27. The antibody of any one of Embodiments 13 to 26, comprising first and second half antibodies, wherein
   a. the first half antibody comprises
      (i) HVR-L1 comprising the amino acid sequence of SEQ ID NO: 9,
      (ii) HVR-L2 comprising the amino acid sequence of SEQ ID NO: 10,
      (iii) HVR-L3 comprising the amino acid sequence of SEQ ID NO: 11,
      (iv) HVR-H1 comprising the amino acid sequence of SEQ ID NO: 12,
      (v) HVR-H2 comprising the amino acid sequence of SEQ ID NO: 13, and
      (vi) HVR-H3 comprising the amino acid sequence of SEQ ID NO: 14, and the second half antibody comprises
      (i) HVR-L1 comprising the amino acid sequence of SEQ ID NO: 74,
      (ii) HVR-L2 comprising the amino acid sequence of SEQ ID NO: 75,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   b. the first half antibody comprises
      (i) HVR-L1 comprising the amino acid sequence of SEQ ID NO: 9,
      (ii) HVR-L2 comprising the amino acid sequence of SEQ ID NO: 10,
      (iii) HVR-L3 comprising the amino acid sequence of SEQ ID NO: 11,
      (iv) HVR-H1 comprising the amino acid sequence of SEQ ID NO: 12,
      (v) HVR-H2 comprising the amino acid sequence of SEQ ID NO: 13, and
      (vi) HVR-H3 comprising the amino acid sequence of SEQ ID NO: 14, and the second half antibody comprises
      (i) HVR-L1 comprising the amino acid sequence of SEQ ID NO: 74,
      (ii) HVR-L2 comprising the amino acid sequence of SEQ ID NO: 77,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   c. the first half antibody comprises
      (i) HVR-L1 comprising the amino acid sequence of SEQ ID NO: 9,
      (ii) HVR-L2 comprising the amino acid sequence of SEQ ID NO: 10,
      (iii) HVR-L3 comprising the amino acid sequence of SEQ ID NO: 11,
      (iv) HVR-H1 comprising the amino acid sequence of SEQ ID NO: 12,
      (v) HVR-H2 comprising the amino acid sequence of SEQ ID NO: 13, and
      (vi) HVR-H3 comprising the amino acid sequence of SEQ ID NO: 14, and the second half antibody comprises
      (i) HVR-L1 comprising the amino acid sequence of SEQ ID NO: 95,
      (ii) HVR-L2 comprising the amino acid sequence of SEQ ID NO: 96,
      (iii) HVR-L3 comprising the amino acid sequence of SEQ ID NO: 97,
      (iv) HVR-H1 comprising the amino acid sequence of SEQ ID NO: 98,
      (v) HVR-H2 comprising the amino acid sequence of SEQ ID NO: 99, and
      (vi) HVR-H3 comprising the amino acid sequence of SEQ ID NO: 100;
   d. the first half antibody comprises
      (i) HVR-L1 comprising the amino acid sequence of SEQ ID NO: 23,
      (ii) HVR-L2 comprising the amino acid sequence of SEQ ID NO: 24,
      (iii) HVR-L3 comprising the amino acid sequence of SEQ ID NO: 25,
      (iv) HVR-H1 comprising the amino acid sequence of SEQ ID NO: 26,
      (v) HVR-H2 comprising the amino acid sequence of SEQ ID NO: 27, and
      (vi) HVR-H3 comprising the amino acid sequence of SEQ ID NO: 28, and the second half antibody comprises
      (i) HVR-L1 comprising the amino acid sequence of SEQ ID NO: 74,
      (ii) HVR-L2 comprising the amino acid sequence of SEQ ID NO: 75,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   e. the first half antibody comprises
      (i) HVR-L1 comprising the amino acid sequence of SEQ ID NO: 23,
      (ii) HVR-L2 comprising the amino acid sequence of SEQ ID NO: 24,
      (iii) HVR-L3 comprising the amino acid sequence of SEQ ID NO: 25,
      (iv) HVR-H1 comprising the amino acid sequence of SEQ ID NO: 26,
      (v) HVR-H2 comprising the amino acid sequence of SEQ ID NO: 27, and
      (vi) HVR-H3 comprising the amino acid sequence of SEQ ID NO: 28, and the second half antibody comprises
      (i) HVR-L1 comprising the amino acid sequence of SEQ ID NO: 74,
      (ii) HVR-L2 comprising the amino acid sequence of SEQ ID NO: 77,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   f. the first half antibody comprises
      (i) HVR-L1 comprising the amino acid sequence of SEQ ID NO: 23,
      (ii) HVR-L2 comprising the amino acid sequence of SEQ ID NO: 24,
      (iii) HVR-L3 comprising the amino acid sequence of SEQ ID NO: 25,
      (iv) HVR-H1 comprising the amino acid sequence of SEQ ID NO: 26,
      (v) HVR-H2 comprising the amino acid sequence of SEQ ID NO: 27, and
      (vi) HVR-H3 comprising the amino acid sequence of SEQ ID NO: 28, and the second half antibody comprises
      (i) HVR-L1 comprising the amino acid sequence of SEQ ID NO: 95,
      (ii) HVR-L2 comprising the amino acid sequence of SEQ ID NO: 96,
      (iii) HVR-L3 comprising the amino acid sequence of SEQ ID NO: 97,
      (iv) HVR-H1 comprising the amino acid sequence of SEQ ID NO: 98, HVR-H2 comprising the amino acid sequence of SEQ ID NO: 99, and
      (v) HVR-H3 comprising the amino acid sequence of SEQ ID NO: 100;
   g. the first half antibody comprises
      (i) HVR-L1 comprising the amino acid sequence of SEQ ID NO: 37,
      (ii) HVR-L2 comprising the amino acid sequence of SEQ ID NO: 38,
      (iii) HVR-L3 comprising the amino acid sequence of SEQ ID NO: 39,
      (iv) HVR-H1 comprising the amino acid sequence of SEQ ID NO: 40,
      (v) HVR-H2 comprising the amino acid sequence of SEQ ID NO: 41, and
      (vi) HVR-H3 comprising the amino acid sequence of SEQ ID NO: 42, and the second half antibody comprises
      (i) HVR-L1 comprising the amino acid sequence of SEQ ID NO: 74,
      (ii) HVR-L2 comprising the amino acid sequence of SEQ ID NO: 75,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   h. the first half antibody comprises
      (i) HVR-L1 comprising the amino acid sequence of SEQ ID NO: 37,
      (ii) HVR-L2 comprising the amino acid sequence of SEQ ID NO: 38,
      (iii) HVR-L3 comprising the amino acid sequence of SEQ ID NO: 39,
      (iv) HVR-H1 comprising the amino acid sequence of SEQ ID NO: 40,
      (v) HVR-H2 comprising the amino acid sequence of SEQ ID NO: 41, and
      (vi) HVR-H3 comprising the amino acid sequence of SEQ ID NO: 42, and the second half antibody comprises
      (i) HVR-L1 comprising the amino acid sequence of SEQ ID NO: 74,
      (ii) HVR-L2 comprising the amino acid sequence of SEQ ID NO: 77,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   i. the first half antibodies comprises
      (i) HVR-L1 comprising the amino acid sequence of SEQ ID NO: 37,
      (ii) HVR-L2 comprising the amino acid sequence of SEQ ID NO: 38,
      (iii) HVR-L3 comprising the amino acid sequence of SEQ ID NO: 39,
      (iv) HVR-H1 comprising the amino acid sequence of SEQ ID NO: 40,
      (v) HVR-H2 comprising the amino acid sequence of SEQ ID NO: 41, and
      (vi) HVR-H3 comprising the amino acid sequence of SEQ ID NO: 42, and the second half antibody comprises
      (i) HVR-L1 comprising the amino acid sequence of SEQ ID NO: 95,
      (ii) HVR-L2 comprising the amino acid sequence of SEQ ID NO: 96,
      (iii) HVR-L3 comprising the amino acid sequence of SEQ ID NO: 97,
      (iv) HVR-H1 comprising the amino acid sequence of SEQ ID NO: 98,
      (v) HVR-H2 comprising the amino acid sequence of SEQ ID NO: 99, and
      (vi) HVR-H3 comprising the amino acid sequence of SEQ ID NO: 100;
   j. the first half antibody comprises
      (i) HVR-L1 comprising the amino acid sequence of SEQ ID NO: 51,
      (ii) HVR-L2 comprising the amino acid sequence of SEQ ID NO: 52,
      (iii) HVR-L3 comprising the amino acid sequence of SEQ ID NO: 53,
      (iv) HVR-H1 comprising the amino acid sequence of SEQ ID NO: 54,
      (v) HVR-H2 comprising the amino acid sequence of SEQ ID NO: 55, and
      (vi) HVR-H3 comprising the amino acid sequence of SEQ ID NO: 56, and the second half antibody comprises
      (i) HVR-L1 comprising the amino acid sequence of SEQ ID NO: 74,
      (ii) HVR-L2 comprising the amino acid sequence of SEQ ID NO: 75,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   k. the first half antibody comprises
      (i) HVR-L1 comprising the amino acid sequence of SEQ ID NO: 51,
      (ii) HVR-L2 comprising the amino acid sequence of SEQ ID NO: 52,
      (iii) HVR-L3 comprising the amino acid sequence of SEQ ID NO: 53,
      (iv) HVR-H1 comprising the amino acid sequence of SEQ ID NO: 54,
      (v) HVR-H2 comprising the amino acid sequence of SEQ ID NO: 55, and
      (vi) HVR-H3 comprising the amino acid sequence of SEQ ID NO: 56, and the second half antibody comprises
      (i) HVR-L1 comprising the amino acid sequence of SEQ ID NO: 74,
      (ii) HVR-L2 comprising the amino acid sequence of SEQ ID NO: 77,
      (iii) HVR-L3 comprising the amino acid sequence of SEQ ID NO: 76,
      (iv) HVR-H1 comprising the amino acid sequence of SEQ ID NO: 78,
      (v) HVR-H2 comprising the amino acid sequence of SEQ ID NO: 79, and
      (vi) HVR-H3 comprising the amino acid sequence of SEQ ID NO: 80;
   l. the first half antibody comprises
      (i) HVR-L1 comprising the amino acid sequence of SEQ ID NO: 51,
      (ii) HVR-L2 comprising the amino acid sequence of SEQ ID NO: 52,
      (iii) HVR-L3 comprising the amino acid sequence of SEQ ID NO: 53,
      (iv) HVR-H1 comprising the amino acid sequence of SEQ ID NO: 54,
      (v) HVR-H2 comprising the amino acid sequence of SEQ ID NO: 55, and
      (vi) HVR-H3 comprising the amino acid sequence of SEQ ID NO: 56, and the other second half antibody comprises
      (i) HVR-L1 comprising the amino acid sequence of SEQ ID NO: 95,
      (ii) HVR-L2 comprising the amino acid sequence of SEQ ID NO: 96,
      (iii) HVR-L3 comprising the amino acid sequence of SEQ ID NO: 97,
      (iv) HVR-H1 comprising the amino acid sequence of SEQ ID NO: 98,
      (v) HVR-H2 comprising the amino acid sequence of SEQ ID NO: 99, and
      (vi) HVR-H3 comprising the amino acid sequence of SEQ ID NO: 100.
Embodiment 28. The antibody of any one of Embodiments 13 to 27, wherein the first half antibody comprises
   a. a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8;
   b. a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22;
   c. a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36; or
   d. a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50.
Embodiment 29. The antibody of any one of Embodiments 13 to 15, 19 to 25 or 27 to 28, wherein the second half antibody comprises
   a. a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73; or
   b. a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73.
Embodiment 30. The antibody of any one of Embodiments 16 to 25 or 26 to 28, wherein the second half antibody comprises
   (i) a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94.
Embodiment 31. The antibody of any one of Embodiments 10 to 27, wherein:
   a. one of the first and second half antibodies comprises a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8,
      (i) and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73;
   b. one of the first and second half antibodies comprises a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8,
      (i) and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73;
   c. one of the first and second half antibodies comprises a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8,
      (i) and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94;
   d. one of the first and second half antibodies comprises a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22,
      (i) and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73;
   e. one of the first and second half antibodies comprises a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22,
      (i) and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73;
   f. one of the first and second half antibodies comprises a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22,
      (i) and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94;
   g. one of the first and second half antibodies comprises a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36,
      (i) and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73;
   h. one of the first and second half antibodies comprises a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36,
      (i) and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73;
   i. one of the first and second half antibodies comprises a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36,
      (i) and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94;
   j. one of the first and second half antibodies comprises a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50,
      (i) and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73;
   k. one of the first and second half antibodies comprises a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50,
      (i) and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73; or
   l. one of the first and second half antibodies comprises a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50,
      (i) and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94.
Embodiment 32. The antibody of any one of Embodiments 13 to 31, wherein the first half antibody comprises
   a. a VL sequence of SEQ ID NO: 7 and a VH sequence of SEQ ID NO: 8;
   b. a VL sequence of SEQ ID NO: 21 and a VH sequence of SEQ ID NO: 22;
   c. a VL sequence of SEQ ID NO: 35 and a VH sequence of SEQ ID NO: 36; or
   d. a VL sequence of SEQ ID NO: 49 and a VH sequence of SEQ ID NO: 50.
Embodiment 33. The antibody of any one of Embodiments 13 to 15, 19 to 25, 27 to 29, or 31 to 32, wherein the second half antibody comprises
   a. a VL sequence of SEQ ID NO: 71 and a VH sequence of SEQ ID NO: 73; or
   b. a VL sequence of SEQ ID NO: 72 and a VH sequence of SEQ ID NO: 73.
Embodiment 34. The antibody of any one of Embodiments 16 to 24, 26 to 28, or 30 to 32, wherein the second half antibody comprises
   (i) a VL sequence of SEQ ID NO: 93 and a VH sequence of SEQ ID NO: 94.
Embodiment 35. The antibody of any one of Embodiments 13 to 34, wherein:
   a. one of the first and second half antibodies comprises a VL sequence of SEQ ID NO: 7 and a VH sequence of SEQ ID NO: 8,
      (i) and the other of the first and second half antibodies comprises a VL sequence of SEQ ID NO: 71 and a VH sequence of SEQ ID NO: 73;
   b. one of the first and second half antibodies comprises a VL sequence of SEQ ID NO: 7 and a VH sequence of SEQ ID NO: 8,
      (i) and the other of the first and second half antibodies comprises a VL sequence of SEQ ID NO: 72 and a VH sequence of SEQ ID NO: 73;
   c. one of the first and second half antibodies comprises a VL sequence of SEQ ID NO: 7 and a VH sequence of SEQ ID NO: 8,
      (i) and the other of the first and second half antibodies comprises a VL sequence of SEQ ID NO: 93 and a VH sequence of SEQ ID NO: 94;
   d. one of the first and second half antibodies comprises a VL sequence of SEQ ID NO: 21 and a VH sequence of SEQ ID NO: 22,
      (i) and the other of the first and second half antibodies comprises a VL sequence of SEQ ID NO: 71 and a VH sequence of SEQ ID NO: 73;
   e. one of the first and second half antibodies comprises a VL sequence of SEQ ID NO: 21 and a VH sequence of SEQ ID NO: 22,
      (i) and the other of the first and second half antibodies comprises a VL sequence of SEQ ID NO: 72 and a VH sequence of SEQ ID NO: 73;
   f. one of the first and second half antibodies comprises a VL sequence of SEQ ID NO: 21 and a VH sequence of SEQ ID NO: 22,
      (i) and the other of the first and second half antibodies comprises a VL sequence of SEQ ID NO: 93 and a VH sequence of SEQ ID NO: 94;
   g. one of the first and second half antibodies comprises a VL sequence of SEQ ID NO: 35 and a VH sequence of SEQ ID NO: 36,
      (i) and the other of the first and second half antibodies comprises a VL sequence of SEQ ID NO: 71 and a VH sequence of SEQ ID NO: 73;
   h. one of the first and second half antibodies comprises a VL sequence of SEQ ID NO: 35 and a VH sequence of SEQ ID NO: 36,
      (i) and the other of the first and second half antibodies comprises a VL sequence of SEQ ID NO: 72 and a VH sequence of SEQ ID NO: 73;
   i. one of the first and second half antibodies comprises a VL sequence of SEQ ID NO: 35 and a VH sequence of SEQ ID NO: 36,
      (i) and the other of the first and second half antibodies comprises a VL sequence of SEQ ID NO: 93 and a VH sequence of SEQ ID NO: 94;
   j. one of the first and second half antibodies comprises a VL sequence of SEQ ID NO: 49 and a VH sequence of SEQ ID NO: 50,
      (i) and the other of the first and second half antibodies comprises a VL sequence of SEQ ID NO: 71 and a VH sequence of SEQ ID NO: 73;
   k. one of the first and second half antibodies comprises a VL sequence of SEQ ID NO: 49 and a VH sequence of SEQ ID NO: 50,
      (i) and the other of the first and second half antibodies comprises a VL sequence of SEQ ID NO: 72 and a VH sequence of SEQ ID NO: 73; or
   l. one of the first and second half antibodies comprises a VL sequence of SEQ ID NO: 49 and a VH sequence of SEQ ID NO: 50,
      (i) and the other of the first and second half antibodies comprises a VL sequence of SEQ ID NO: 93 and a VH sequence of SEQ ID NO: 94.
Embodiment 36. The antibody of any one of Embodiments 13 to 35, which is a monoclonal antibody.
Embodiment 37. The antibody of any one of Embodiments 13 to 36, which is a mouse, rabbit, human, humanized, or chimeric antibody.
Embodiment 38. The antibody of any one of Embodiments 13 to 37, wherein the first antigen binding site is human or humanized.
Embodiment 39. The antibody of any one of Embodiments 13 to 38, wherein the second antigen binding site is human or humanized.
Embodiment 40. The antibody of any one of Embodiments 13 to 39, wherein the antibody is an IgG antibody.
Embodiment 41. The antibody of any one of Embodiments 13 to 40, wherein the antibody is an IgG1, IgG2a, IgG2b, IgG3, or IgG4 antibody.
Embodiment 42. The antibody of any one of Embodiments 13 to 41, wherein the antibody is an IgG1 or IgG4 antibody.
Embodiment 43. The antibody of any one of Embodiments 13 to 42, wherein the first half antibody comprises a first heavy chain constant region comprising a knob mutation and the second half antibody comprises a second heavy chain constant region comprising a hole mutation; or wherein the first half antibody comprises a first heavy chain constant region comprising a hole mutation and the second half antibody comprises a second heavy chain constant region comprising a knob mutation.
Embodiment 44. The antibody of Embodiment 43, wherein the antibody is an IgG1 antibody and wherein the knob mutation comprises a T366W mutation.
Embodiment 45. The antibody of Embodiment 43 or Embodiment 44, wherein the antibody is an IgG1 antibody and wherein the hole mutation comprises at least one, at least two, or three, such as one to two, one to three, or two to three mutations selected from T366S, L368A, and Y407V.
Embodiment 46. The antibody of Embodiment 43, wherein the antibody is an IgG4 antibody and wherein the knob mutation comprises a T366W mutation.
Embodiment 47. The antibody of Embodiment 43 or Embodiment 46, wherein the antibody is an IgG4 antibody and wherein the hole mutation comprises at least one, at least two, or three, such as one to two, one to three, or two to three mutations selected from T366S, L368A, and Y407V mutations.
Embodiment 48. The antibody of any one of Embodiments 43 to 47, wherein the first half antibody comprises
   a. a light chain sequence having at least about 95%, such as 96%, 97%, 98%, 99%, or 100% sequence identity to SEQ ID NO: 2;
   b. a light chain sequence having at least about 95%, such as 96%, 97%, 98%, 99%, or 100% sequence identity to SEQ ID NO: 16;
   c. a light chain sequence having at least about 95%, such as 96%, 97%, 98%, 99%, or 100% sequence identity to SEQ ID NO: 30; or
   d. a light chain sequence having at least about 95%, such as 96%, 97%, 98%, 99%, or 100% sequence identity to SEQ ID NO: 44.
Embodiment 49. The antibody of any one of the Embodiments 13 to 48, wherein the first half antibody comprises
   a. a heavy chain sequence having at least about 95%, such as 96%, 97%, 98%, 99%, or 100% sequence identity to SEQ ID NO: 4;
   b. a heavy chain sequence having at least about 95%, such as 96%, 97%, 98%, 99%, or 100% sequence identity to SEQ ID NO: 18;
   c. a heavy chain sequence having at least about 95%, such as 96%, 97%, 98%, 99%, or 100% sequence identity to SEQ ID NO: 32; or
   d. a heavy chain sequence having at least about 95%, such as 96%, 97%, 98%, 99%, or 100% sequence identity to SEQ ID NO: 46; optionally wherein a C-terminal lysine is missing from one or more heavy chains.
Embodiment 50. The antibody of any one of Embodiments 13 to 49, wherein the first half antibody comprises
   a. a light chain sequence having at least about 95%, such as 96%, 97%, 98%, 99%, or 100% sequence identity to SEQ ID NO: 2 and a heavy chain sequence having at least about 95%, such as 96%, 97%, 98%, 99%, or 100% sequence identity to SEQ ID NO: 4;
   b. a light chain sequence having at least about 95%, such as 96%, 97%, 98%, 99%, or 100% sequence identity to SEQ ID NO: 16 and a heavy chain sequence having at least about 95%, such as 96%, 97%, 98%, 99%, or 100% sequence identity to SEQ ID NO: 18;
   c. a light chain sequence having at least about 95%, such as 96%, 97%, 98%, 99%, or 100% sequence identity to SEQ ID NO: 30 and a heavy chain sequence having at least about 95%, such as 96%, 97%, 98%, 99%, or 100% sequence identity to SEQ ID NO: 32; or
   d. a light chain sequence having at least about 95%, such as 96%, 97%, 98%, 99%, or 100% sequence identity to SEQ ID NO: 44 and a heavy chain sequence having at least about 95%, such as 96%, 97%, 98%, 99%, or 100% sequence identity to SEQ ID NO: 46; optionally wherein a C-terminal lysine is missing from one or more heavy chains.
Embodiment 51. The antibody of any one of Embodiments 13 to 48, wherein the first half antibody comprises
   a. a heavy chain sequence having at least about 95%, such as 96%, 97%, 98%, 99%, or 100% sequence identity to SEQ ID NO: 6;
   b. a heavy chain sequence having at least about 95%, such as 96%, 97%, 98%, 99%, or 100% sequence identity to SEQ ID NO: 20;
   c. a heavy chain sequence having at least about 95%, such as 96%, 97%, 98%, 99%, or 100% sequence identity to SEQ ID NO: 34; or
   d. a heavy chain sequence having at least about 95%, such as 96%, 97%, 98%, 99%, or 100% sequence identity to SEQ ID NO: 48; optionally wherein a C-terminal lysine is missing from one or more heavy chains.
Embodiment 52. The antibody of any one of Embodiments 13 to 48 or 51, wherein the first half antibody comprises
   a. a light chain sequence having at least about 95%, such as 96%, 97%, 98%, 99%, or 100% sequence identity to SEQ ID NO: 2 and a heavy chain sequence having at least about 95%, such as 96%, 97%, 98%, 99%, or 100% sequence identity to SEQ ID NO: 6;
   b. a light chain sequence having at least about 95%, such as 96%, 97%, 98%, 99%, or 100% sequence identity to SEQ ID NO: 16 and a heavy chain sequence having at least about 95%, such as 96%, 97%, 98%, 99%, or 100% sequence identity to SEQ ID NO: 20;
   c. a light chain sequence having at least about 95%, such as 96%, 97%, 98%, 99%, or 100% sequence identity to SEQ ID NO: 30 and a heavy chain sequence having at least about 95%, such as 96%, 97%, 98%, 99%, or 100% sequence identity to SEQ ID NO: 34; or
   d. a light chain sequence having at least about 95%, such as 96%, 97%, 98%, 99%, or 100% sequence identity to SEQ ID NO: 44 and a heavy chain sequence having at least about 95%, such as 96%, 97%, 98%, 99%, or 100% sequence identity to SEQ ID NO: 48; optionally wherein a C-terminal lysine is missing from one or more heavy chains.
Embodiment 53. The antibody of any one of Embodiments 13 to 15, 19 to 25, 27 to 29, 31 to 33, or 35 to 52, wherein the second half antibody comprises a light chain sequence having at least about 95%, such as 96%, 97%, 98%, 99%, or 100% sequence identity to SEQ ID NO: 60.
Embodiment 54. The antibody of any one of Embodiments 13 to 15, 19 to 15, 27 to 29, 31 to 33, or 35 to 53, wherein the second half antibody comprises a heavy chain sequence having at least about 95%, such as 96%, 97%, 98%, 99%, or 100% sequence identity to SEQ ID NO: 68; optionally wherein a C-terminal lysine is missing from the heavy chain.
Embodiment 55. The antibody of any one of Embodiments 13 to 15, 19 to 25, 27 to 29, 31 to 33, or 35 to 54, wherein the second half antibody comprises a light chain sequence having at least about 95%, such as 96%, 97%, 98%, 99%, or 100% sequence identity to SEQ ID NO: 60 and a heavy chain sequence having at least 95%, such as 96%, 97%, 98%, 99%, or 100% sequence identity to SEQ ID NO: 68; optionally wherein a C-terminal lysine is missing from one or more heavy chains.
Embodiment 56. The antibody of any one of Embodiments 13 to 15, 19 to 25, 27 to 29, 31 to 33, or 33 to 53, wherein the second half antibody comprises a heavy chain sequence having at least about 95%, such as 96%, 97%, 98%, 99%, or 100% sequence identity to SEQ ID NO: 70; optionally wherein a C-terminal lysine is missing from the heavy chain.
Embodiment 57. The antibody of any one of Embodiments 13 to 15, 19 to 25, 27 to 29, 31 to 33, 35 to 53 or 56, wherein the second half antibody comprises a light chain sequence having at least about 95%, such as 96%, 97%, 98%, 99%, or 100% sequence identity to SEQ ID NO: 60 and a heavy chain sequence having at least 95%, such as 96%, 97%, 98%, 99%, or 100% sequence identity to SEQ ID NO: 70; optionally wherein a C-terminal lysine is missing from one or more heavy chains.
Embodiment 58. The antibody of any one of Embodiments 16 to 24, 26 to 28, 30 to 32, or 34 to 52, wherein the second half antibody comprises a light chain sequence having at least about 95%, such as 96%, 97%, 98%, 99%, or 100% sequence identity to SEQ ID NO: 84.
Embodiment 59. The antibody of any one of Embodiments 16 to 24, 26 to 28, 30 to 31, 34 to 52 or 5528 wherein the second half antibody comprises a heavy chain sequence having at least about 95%, such as 96%, 97%, 98%, 99%, or 100% sequence identity to SEQ ID NO: 90; optionally wherein a C-terminal lysine is missing from one or more heavy chains.
Embodiment 60. The antibody of any one of Embodiments 16 to 24, 26 to 28, 30 to 32, 34 to 52, or 58 to 59, wherein the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 90; optionally wherein a C-terminal lysine is missing from one or more heavy chains.
Embodiment 61. The antibody of any one of Embodiments 16 to 24, 26 to 28, 30 to 32, 34 to 52 or 58, wherein the second half antibody comprises a heavy chain sequence having at least about 95%, such as 96%, 97%, 98%, 99%, or 100% sequence identity to SEQ ID NO: 92; optionally wherein a C-terminal lysine is missing from one or more heavy chains.
Embodiment 62. The antibody of any one of Embodiments 16 to 24, 26 to 28, 30 to 32, 33 to 52, 58 or 61, wherein the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 92; optionally wherein a C-terminal lysine is missing from one or more heavy chains.
Embodiment 63. The antibody of any one of Embodiments 13 to 15, 19 to 25, 27 to 29, 31 to 33, or 35 to 57, wherein:
   a. the first half antibody comprises a light chain sequence having at least about 95%, such as 96%, 97%, 98%, 99%, or 100% sequence identity to SEQ ID NO: 2 and a heavy chain sequence having at least about 95%, such as 96%, 97%, 98%, 99%, or 100% sequence identity to SEQ ID NO: 4, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 70;
   b. the first half antibody comprises a light chain sequence having at least about 95%, such as 96%, 97%, 98%, 99%, or 100% sequence identity to SEQ ID NO: 2 and a heavy chain sequence having at least about 95%, such as 96%, 97%, 98%, 99%, or 100% sequence identity to SEQ ID NO: 6, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 68;
   c. the first half antibody comprises a light chain sequence having at least about 95%, such as 96%, 97%, 98%, 99%, or 100% sequence identity to SEQ ID NO: 16 and a heavy chain sequence having at least about 95%, such as 96%, 97%, 98%, 99%, or 100% sequence identity to SEQ ID NO: 18, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 70;
   d. the first half antibody comprises a light chain sequence having at least about 95%, such as 96%, 97%, 98%, 99%, or 100% sequence identity to SEQ ID NO: 16 and a heavy chain sequence having at least about 95%, such as 96%, 97%, 98%, 99%, or 100% sequence identity to SEQ ID NO: 20, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 68;
   e. the first half antibody comprises a light chain sequence having at least about 95%, such as 96%, 97%, 98%, 99%, or 100% sequence identity to SEQ ID NO: 30 and a heavy chain sequence having at least about 95%, such as 96%, 97%, 98%, 99%, or 100% sequence identity to SEQ ID NO: 32, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 70;
   f. chain sequence having at least about 95%, such as 96%, 97%, 98%, 99%, or 100% sequence identity to SEQ ID NO: 30 and a heavy chain sequence having at least about 95%, such as 96%, 97%, 98%, 99%, or 100% sequence identity to SEQ ID NO: 34, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 68;
   g. the first half antibody comprises a light chain sequence having at least about 95%, such as 96%, 97%, 98%, 99%, or 100% sequence identity to SEQ ID NO: 44 and a heavy chain sequence having at least about 95%, such as 96%, 97%, 98%, 99%, or 100% sequence identity to SEQ ID NO: 46, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 70;
   h. the first half antibody comprises a light chain sequence having at least about 95%, such as 96%, 97%, 98%, 99%, or 100% sequence identity to SEQ ID NO: 44 and a heavy chain sequence having at least about 95%, such as 96%, 97%, 98%, 99%, or 100% sequence identity to SEQ ID NO: 48, and the second half antibody comprises a light chain sequence having at least about 95%, such as 96%, 97%, 98%, 99%, or 100% sequence identity to SEQ ID NO: 60 and a heavy chain sequence having at least about 95%, such as 96%, 97%, 98%, 99%, or 100% sequence identity to SEQ ID NO: 68, optionally wherein a C-terminal lysine is missing from one or more heavy chains.
Embodiment 64. The antibody of any one of Embodiments 16 to 24, 26 to 28, 30 to 32, 34 to 52, or 58 to 62, wherein:
   a. the first half antibody comprises a light chain sequence having at least about 95%, such as 96%, 97%, 98%, 99%, or 100% sequence identity to SEQ ID NO: 2 and a heavy chain sequence having at least about 95%, such as 96%, 97%, 98%, 99%, or 100% sequence identity to SEQ ID NO: 4, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 92;
   b. the first half antibody comprises a light chain sequence having at least about 95%, such as 96%, 97%, 98%, 99%, or 100% sequence identity to SEQ ID NO: 2 and a heavy chain sequence having at least about 95%, such as 96%, 97%, 98%, 99%, or 100% sequence identity to SEQ ID NO: 6, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 90;
   c. the first half antibody comprises a light chain sequence having at least about 95%, such as 96%, 97%, 98%, 99%, or 100% sequence identity to SEQ ID NO: 16 and a heavy chain sequence having at least about 95%, such as 96%, 97%, 98%, 99%, or 100% sequence identity to SEQ ID NO: 18, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 92;
   d. the first half antibody comprises a light chain sequence having at least about 95%, such as 96%, 97%, 98%, 99%, or 100% sequence identity to SEQ ID NO: 16 and a heavy chain sequence having at least about 95%, such as 96%, 97%, 98%, 99%, or 100% sequence identity to SEQ ID NO: 20, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 90;
   e. the first half antibody comprises a light chain sequence having at least about 95%, such as 96%, 97%, 98%, 99%, or 100% sequence identity to SEQ ID NO: 30 and a heavy chain sequence having at least about 95%, such as 96%, 97%, 98%, 99%, or 100% sequence identity to SEQ ID NO: 32, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 92;
   f. the first half antibody comprises a light chain sequence having at least about 95%, such as 96%, 97%, 98%, 99%, or 100% sequence identity to SEQ ID NO: 30 and a heavy chain sequence having at least about 95%, such as 96%, 97%, 98%, 99%, or 100% sequence identity to SEQ ID NO: 34, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 90;
   g. the first half antibody comprises a light chain sequence having at least about 95%, such as 96%, 97%, 98%, 99%, or 100% sequence identity to SEQ ID NO: 44 and a heavy chain sequence having at least about 95%, such as 96%, 97%, 98%, 99%, or 100% sequence identity to SEQ ID NO: 46, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 92;
   h. the first half antibody comprises a light chain sequence having at least about 95%, such as 96%, 97%, 98%, 99%, or 100% sequence identity to SEQ ID NO: 44 and a heavy chain sequence having at least about 95%, such as 96%, 97%, 98%, 99%, or 100% sequence identity to SEQ ID NO: 48, and the second half antibody comprises a light chain sequence having at least about 95%, such as 96%, 97%, 98%, 99%, or 100% sequence identity to SEQ ID NO: 84 and a heavy chain sequence having at least about 95%, such as 96%, 97%, 98%, 99%, or 100% sequence identity to SEQ ID NO: 90, optionally wherein a C-terminal lysine is missing from one or more heavy chains.
Embodiment 65. The antibody of any one of Embodiments 13 to 64, wherein the first half antibody comprises
   a. a light chain sequence of SEQ ID NO: 2;
   b. a light chain sequence of SEQ ID NO: 16;
   c. a light chain sequence of SEQ ID NO: 30; or
   d. a light chain sequence of SEQ ID NO: 44.
Embodiment 66. The antibody of any one of Embodiments 13 to 65, wherein the first half antibody comprises
   a. a heavy chain sequence of SEQ ID NO: 4;
   b. a heavy chain sequence of SEQ ID NO: 18;
   c. a heavy chain sequence of SEQ ID NO: 32; or
   d. a heavy chain sequence of SEQ ID NO: 46; optionally wherein a C-terminal lysine is missing from one or more heavy chains.
Embodiment 67. The antibody of any one of Embodiments 13 to 66, wherein the first half antibody comprises
   a. a light chain sequence of SEQ ID NO: 2 and a heavy chain sequence of SEQ ID NO: 4;
   b. a light chain sequence of SEQ ID NO: 16 and a heavy chain sequence of SEQ ID NO: 18;
   c. a light chain sequence of SEQ ID NO: 30 and a heavy chain sequence of SEQ ID NO: 32; or
   d. a light chain sequence of SEQ ID NO: 44 and a heavy chain sequence of SEQ ID NO: 46; optionally wherein a C-terminal lysine is missing from one or more heavy chains.
Embodiment 68. The antibody of any one of Embodiments 13 to 65, wherein the first half antibody comprises
   a. a heavy chain sequence of SEQ ID NO: 6;
   b. a heavy chain sequence of SEQ ID NO: 20;
   c. a heavy chain sequence of SEQ ID NO: 34; or
   d. a heavy chain sequence of SEQ ID NO: 48; optionally wherein a C-terminal lysine is missing from one or more heavy chains.
Embodiment 69. The antibody of any one of Embodiments 13 to 65 or 68, wherein the first half antibody comprises
   a. a light chain sequence of SEQ ID NO: 2 and a heavy chain sequence of SEQ ID NO: 6;
   b. a light chain sequence of SEQ ID NO: 16 and a heavy chain sequence of SEQ ID NO: 20;
   c. a light chain sequence of SEQ ID NO: 30 and a heavy chain sequence of SEQ ID NO: 34; or
   d. a light chain sequence of SEQ ID NO: 44 and a heavy chain sequence of SEQ ID NO: 48; optionally wherein a C-terminal lysine is missing from one or more heavy chains.
Embodiment 70. The antibody of any one of Embodiments 13 to 15, 19 to 25, 27 to 29, 31 to 33, 35 to 53, 63, or 65 to 69, wherein the second half antibody comprises a light chain sequence of SEQ ID NO: 60.
Embodiment 71. The antibody of any one of Embodiments 13 to 15, 19 to 25, 27 to 29, 31 to 33, 35 to 54, 63, or 65 to 70, wherein the second half antibody comprises a heavy chain sequence of SEQ ID NO: 68; optionally wherein a C-terminal lysine is missing from the heavy chain.
Embodiment 72. The antibody of any one of Embodiments 13 to 15, 19 to 25, 27 to 29, 31 to 33, 35 to 55, 63, or 65 to 71, wherein the second half antibody comprises a light chain sequence of SEQ ID NO: 60 and a heavy chain sequence of SEQ ID NO: 68; optionally wherein a C-terminal lysine is missing from one or more heavy chains.
Embodiment 73. The antibody of any one of Embodiments 13 to 15, 19 to 25, 27 to 29, 31 to 33, 35 to 54, 63, or 65 to 70, wherein the second half antibody comprises a heavy chain sequence of SEQ ID NO: 70; optionally wherein a C-terminal lysine is missing from the heavy chain.
Embodiment 74. The antibody of any one of Embodiments 13 to 15, 19 to 25, 27 to 29, 31 to 33, 35 to 54, 56, 63, 65 to 70 or 73, wherein the second half antibody comprises a light chain sequence of SEQ ID NO: 60 and a heavy chain sequence of SEQ ID NO: 70; optionally wherein a C-terminal lysine is missing from one or more heavy chains.
Embodiment 75. The antibody of any one of Embodiments 16 to 24, 26 to 28, 30 to 32, 34 to 52, 58, or 64 to 69, wherein the second half antibody comprises a light chain sequence of SEQ ID NO: 84.
Embodiment 76. The antibody of any one of Embodiments 16 to 24, 26 to 28, 30 to 32, 34 to 52, 58 to 59, 64 to 69 or 75, wherein the second half antibody comprises a heavy chain sequence of SEQ ID NO: 90; optionally wherein a C-terminal lysine is missing from one or more heavy chains.
Embodiment 77. The antibody of any one of Embodiments 16 to 24, 26 to 28, 30 to 32, 34 to 52, 58 to 60, 64 to 69 or 75 to 76, wherein the second half antibody comprises a light chain sequence of SEQ ID NO: 84 and a heavy chain sequence of SEQ ID NO: 90; optionally wherein a C-terminal lysine is missing from one or more heavy chains.
Embodiment 78. The antibody of any one of Embodiments 16 to 24, 26 to 28, 30 to 32, 34 to 52, 58, 61, 64 to 69 or 75, wherein the second half antibody comprises a heavy chain of SEQ ID NO: 92; optionally wherein a C-terminal lysine is missing from one or more heavy chains.
Embodiment 79. The antibody of any one of Embodiments 16 to 24, 26 to 28, 30 to 32, 34 to 52, 58, 61 to 62, 64 to 69, 75, or 78, wherein the second half antibody comprises a light chain sequence of SEQ ID NO: 84 and a heavy chain sequence of SEQ ID NO: 92; optionally wherein a C-terminal lysine is missing from one or more heavy chains.
Embodiment 80. The antibody of any one of Embodiments 16 to 15, 19 to 25, 27 to 29, 31 to 33, 35 to 57, 63, or 65 to 74, wherein:
   a. the first half antibody comprises a light chain sequence of SEQ ID NO: 2 and a heavy chain sequence of SEQ ID NO: 4, and the second half antibody comprises a light chain sequence of SEQ ID NO: 60 and a heavy chain sequence of SEQ ID NO: 70;
   b. the first half antibody comprises a light chain sequence of SEQ ID NO: 2 and a heavy chain sequence of SEQ ID NO: 6, and the second half antibody comprises a light chain sequence of SEQ ID NO: 60 and a heavy chain sequence of SEQ ID NO: 68;
   c. the first half antibody comprises a light chain sequence of SEQ ID NO: 16 and a heavy chain sequence of SEQ ID NO: 18, and the second half antibody comprises a light chain sequence of SEQ ID NO: 60 and a heavy chain sequence of SEQ ID NO: 70;
   d. the first half antibody comprises a light chain sequence of SEQ ID NO: 16 and a heavy chain sequence of SEQ ID NO: 20, and the second half antibody comprises a light chain sequence of SEQ ID NO: 60 and a heavy chain sequence of SEQ ID NO: 68;
   e. the first half antibody comprises a light chain sequence of SEQ ID NO: 30 and a heavy chain sequence of SEQ ID NO: 32, and the second half antibody comprises a light chain sequence of SEQ ID NO: 60 and a heavy chain sequence of SEQ ID NO: 70;
   f. the first half antibody comprises a light chain sequence of SEQ ID NO: 30 and a heavy chain sequence of SEQ ID NO: 34, and the second half antibody comprises a light chain sequence of SEQ ID NO: 60 and a heavy chain sequence of SEQ ID NO: 68;
   g. the first half antibody comprises a light chain sequence of SEQ ID NO: 44 and a heavy chain sequence of SEQ ID NO: 46, and the second half antibody comprises a light chain sequence of SEQ ID NO: 60 and a heavy chain sequence of SEQ ID NO: 70;
   h. the first half antibody comprises a light chain sequence of SEQ ID NO: 44 and a heavy chain sequence of SEQ ID NO: 48, and the second half antibody comprises a light chain sequence of SEQ ID NO: 60 and a heavy chain sequence of SEQ ID NO: 68, optionally wherein a C-terminal lysine is missing from one or more heavy chains.
Embodiment 81. The antibody of any one of Embodiments 16 to 24, 26 to 28, 30 to 32, 34 to 52, 58 to 62, 64 to 69, or 75 to 79, wherein:
   a. the first half antibody comprises a light chain sequence of SEQ ID NO: 2 and a heavy chain sequence of SEQ ID NO: 4, and the second half antibody comprises a light chain sequence of SEQ ID NO: 84 and a heavy chain sequence of SEQ ID NO: 92;
   b. the first half antibody comprises a light chain sequence of SEQ ID NO: 2 and a heavy chain sequence of SEQ ID NO: 6, and the second half antibody comprises a light chain sequence of SEQ ID NO: 84 and a heavy chain sequence of SEQ ID NO: 90;
   c. the first half antibody comprises a light chain sequence of SEQ ID NO: 16 and a heavy chain sequence of SEQ ID NO: 18, and the second half antibody comprises a light chain sequence of SEQ ID NO: 84 and a heavy chain sequence of SEQ ID NO: 92;
   d. the first half antibody comprises a light chain sequence of SEQ ID NO: 16 and a heavy chain sequence of SEQ ID NO: 20, and the second half antibody comprises a light chain sequence of SEQ ID NO: 84 and a heavy chain sequence of SEQ ID NO: 90;
   e. the first half antibody comprises a light chain sequence of SEQ ID NO: 30 and a heavy chain sequence of SEQ ID NO: 32, and the second half antibody comprises a light chain sequence of SEQ ID NO: 84 and a heavy chain sequence of SEQ ID NO: 92;
   f. chain sequence of SEQ ID NO: 30 and a heavy chain sequence of SEQ ID NO: 34, and the second half antibody comprises a light chain sequence of SEQ ID NO: 84 and a heavy chain sequence of SEQ ID NO: 90;
   g. the first half antibody comprises a light chain sequence of SEQ ID NO: 44 and a heavy chain sequence of SEQ ID NO: 46, and the second half antibody comprises a light chain sequence of SEQ ID NO: 84 and a heavy chain sequence of SEQ ID NO: 92;
   h. the first half antibody comprises a light chain sequence of SEQ ID NO: 44 and a heavy chain sequence of SEQ ID NO: 48, and the second half antibody comprises a light chain sequence of SEQ ID NO: 84 and a heavy chain sequence of SEQ ID NO: 90, optionally wherein a C-terminal lysine is missing from one or more heavy chains.
Embodiment 82. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 2 and a heavy chain sequence of SEQ ID NO: 4, and the second half antibody comprises a light chain sequence of SEQ ID NO: 60 and a heavy chain sequence of SEQ ID NO: 70, and optionally wherein a C-terminal lysine is missing from one or more heavy chains.
Embodiment 83. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 2 and a heavy chain sequence of SEQ ID NO: 6, and the second half antibody comprises a light chain sequence of SEQ ID NO: 60 and a heavy chain sequence of SEQ ID NO: 68, and optionally wherein a C-terminal lysine is missing from one or more heavy chains.
Embodiment 84. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 16 and a heavy chain sequence of SEQ ID NO: 18, and the second half antibody comprises a light chain sequence of SEQ ID NO: 60 and a heavy chain sequence of SEQ ID NO: 70, and optionally wherein a C-terminal lysine is missing from one or more heavy chains.
Embodiment 85. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 16 and a heavy chain sequence of SEQ ID NO: 20, and the second half antibody comprises a light chain sequence of SEQ ID NO: 60 and a heavy chain sequence of SEQ ID NO: 68, and optionally wherein a C-terminal lysine is missing from one or more heavy chains.
Embodiment 86. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 30 and a heavy chain sequence of SEQ ID NO: 32, and the second half antibody comprises a light chain sequence of SEQ ID NO: 60 and a heavy chain sequence of SEQ ID NO: 70, and optionally wherein a C-terminal lysine is missing from one or more heavy chains.
Embodiment 87. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 30 and a heavy chain sequence of SEQ ID NO: 34, and the second half antibody comprises a light chain sequence of SEQ ID NO: 60 and a heavy chain sequence of SEQ ID NO: 68, and optionally wherein a C-terminal lysine is missing from one or more heavy chains.
Embodiment 88. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 44 and a heavy chain sequence of SEQ ID NO: 46, and the second half antibody comprises a light chain sequence of SEQ ID NO: 60 and a heavy chain sequence of SEQ ID NO: 70, and optionally wherein a C-terminal lysine is missing from one or more heavy chains.
Embodiment 89. The antibody of any one of Embodiments 13 to 15, 19 to 25, 27 to 29, 31 to 33, 35 to 57, 63, 65 to 74 or 80, wherein the first half antibody comprises a light chain sequence of SEQ ID NO: 44 and a heavy chain sequence of SEQ ID NO: 48, and the second half antibody comprises a light chain sequence of SEQ ID NO: 60 and a heavy chain sequence of SEQ ID NO: 68, and optionally wherein a C-terminal lysine is missing from one or more heavy chains.
Embodiment 90. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 2 and a heavy chain sequence of SEQ ID NO: 4, and the second half antibody comprises a light chain sequence of SEQ ID NO: 84 and a heavy chain sequence of SEQ ID NO: 92, and optionally wherein a C-terminal lysine is missing from one or more heavy chains.
Embodiment 91. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 2 and a heavy chain sequence of SEQ ID NO: 6, and the second half antibody comprises a light chain sequence of SEQ ID NO: 84 and a heavy chain sequence of SEQ ID NO: 90, and optionally wherein a C-terminal lysine is missing from one or more heavy chains.
Embodiment 92. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 16 and a heavy chain sequence of SEQ ID NO: 18, and the second half antibody comprises a light chain sequence of SEQ ID NO: 84 and a heavy chain sequence of SEQ ID NO: 92, and optionally wherein a C-terminal lysine is missing from one or more heavy chains.
Embodiment 93. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 16 and a heavy chain sequence of SEQ ID NO: 20, and the second half antibody comprises a light chain sequence of SEQ ID NO: 84 and a heavy chain sequence of SEQ ID NO: 90, and optionally wherein a C-terminal lysine is missing from one or more heavy chains.
Embodiment 94. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 30 and a heavy chain sequence of SEQ ID NO: 32, and the second half antibody comprises a light chain sequence of SEQ ID NO: 84 and a heavy chain sequence of SEQ ID NO: 92, and optionally wherein a C-terminal lysine is missing from one or more heavy chains.
Embodiment 95. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 30 and a heavy chain sequence of SEQ ID NO: 34, and the second half antibody comprises a light chain sequence of SEQ ID NO: 84 and a heavy chain sequence of SEQ ID NO: 90, and optionally wherein a C-terminal lysine is missing from one or more heavy chains.
Embodiment 96. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 44 and a heavy chain sequence of SEQ ID NO: 46, and the second half antibody comprises a light chain sequence of SEQ ID NO: 84 and a heavy chain sequence of SEQ ID NO: 92, and optionally wherein a C-terminal lysine is missing from one or more heavy chains.
Embodiment 97. The antibody of any one of Embodiments 16 to 24, 26 to 28, 30 to 32, 34 to 52, 58 to 62, 64 to 69, 75 to 79 or 81, wherein the first half antibody comprises a light chain sequence of SEQ ID NO: 44 and a heavy chain sequence of SEQ ID NO: 48, and the second half antibody comprises a light chain sequence of SEQ ID NO: 84 and a heavy chain sequence of SEQ ID NO: 90, and optionally wherein a C-terminal lysine is missing from one or more heavy chains.
Embodiment 98. The antibody of any one of Embodiments 13 to 97, which is a bispecific antibody.
Embodiment 99. The antibody of any one of Embodiments 13 to 98, which is a diabody, triabody, or tetrabody.
Embodiment 100. The antibody of any one of Embodiments 13 to 99, conjugated to a label.
Embodiment 101. The antibody of Embodiment 100, wherein the label is a fluorescent, enzymatic, or chromogenic label.
Embodiment 102. The antibody of Embodiment 100, wherein the label is a radioisotope, which is optionally a positron emitter, which is optionally ⁸⁹Zr.
Embodiment 103. A composition comprising the antibody of any one of Embodiments 13 to 102, wherein the composition is substantially free of monospecific antibodies, unassembled half antibodies, or both monospecific antibodies and unassembled half antibodies.
Embodiment 104. An immunoconjugate comprising the antibody of any one of Embodiments 13 to 102 and a cytotoxic agent or an anti-inflammatory agent.
Embodiment 105. A pharmaceutical formulation comprising a pharmaceutically acceptable carrier and at least one of:
   a) the antibody of any one of Embodiments 13 to 102; or
   b) the immunoconjugate of Embodiment 104;
   b. optionally wherein the composition is substantially free of monospecific antibodies, unassembled half antibodies, or both monospecific antibodies and unassembled half antibodies.
Embodiment 106. The pharmaceutical formulation of Embodiment 105, further comprising an additional therapeutic agent.
Embodiment 107. An isolated nucleic acid encoding the antibody of any one of Embodiments 13 to 102.
Embodiment 108. A vector comprising the nucleic acid of Embodiment 107.
Embodiment 109. A host cell comprising the nucleic acid of Embodiment 107.
Embodiment 110. A method of producing an antibody comprising culturing the host cell of Embodiment 106 under conditions wherein the antibody is produced.
Embodiment 111. The method of Embodiment 110, further comprising recovering the antibody from the host cell.
Embodiment 112. A method of making the antibody of any one of Embodiments 13 to 102, comprising forming the antibody from a first half antibody and a second half antibody.
Embodiment 113. The antibody of any one of Embodiments 13 to 102 for use as a medicament.
Embodiment 114. A method of determining the presence of a polyubiquitinated protein in a sample suspected of containing a polyubiquitin or polyubiquitinated protein, comprising exposing the sample to the antibody of any of Embodiments 13 to 102 and determining the binding of the antibody to a polyubiquitinated protein in the sample.
Embodiment 115. A method of separating K11-linked polyubiquitinated protein from non-K11-linked polyubiquitinated protein in a sample, comprising contacting the sample with the antibody of any of Embodiments 13 to 102.
Embodiment 116. A method of separating K48-linked polyubiquitinated protein from non-K48-linked polyubiquitinated protein in a sample, comprising contacting the sample with the antibody of any of Embodiments 13 to 102.
Embodiment 117. A method of separating K63-linked polyubiquitinated protein from non-K63-linked polyubiquitinated protein in a sample, comprising contacting the sample with the antibody of any of Embodiments 13 to 102.
Embodiment 118. A method of separating M1-linked polyubiquitinated protein from non-M1-linked polyubiquitinated protein in a sample, comprising contacting the sample with the antibody of any of Embodiments 13 to 102.
Embodiment 119. A method of determining the function and/or activity of a polyubiquitinated protein in a cell or sample comprising contacting the cell or sample with the antibody of any of Embodiments 13 to 102 and assessing the effect of said contacting step on the cell or sample.
Embodiment 120. The method of any one of Embodiments 114 to 119, wherein the polyubiquitinated protein comprises RIP1.
Embodiment 121. The method of any one of Embodiments 114 to 119, wherein the polyubiquitinated protein comprises RIP2.
Embodiment 122. A method of determining the presence of a polyubiquitinated protein in a sample suspected of containing a polyubiquitinated protein, wherein the polyubiquitinated protein is a pro-inflammatory protein and comprises a polyubiquitin, comprising exposing the sample to the antibody of any of Embodiments 10 to 99.
Embodiment 123. The method of Embodiment 122, wherein the polyubiquitinated protein comprises a M1-linked polyubiquitin and/or a K63-linked polyubiquitin.
Embodiment 124. The method of Embodiment 122 or 123, wherein the pro-inflammatory protein is a component of one or more signaling complexes.
Embodiment 125. The method of any one of Embodiments 122 to 124, wherein the pro-inflammatory protein is RIP1.
Embodiment 126. The method of any one of Embodiments 122 to 125, wherein the pro-inflammatory protein is RIP2.
Embodiment 127. The method of any one of Embodiments 122 to 126, wherein the pro-inflammatory protein has an elevated level of ubiquitination in the inflammatory state relative to the level of ubiquitination when not in the inflammatory state.
Embodiment 128. The method of any one of Embodiments 122 to 127, wherein the pro-inflammatory protein has a level of ubiquitination of at least 1-fold, 2-fold, 3-fold, 4-fold, 5-fold, 6-fold, 7-fold, 8-fold, 9-fold, 10-fold, 11-fold, 12-fold, 1-fold to 12-fold, 2-fold to 12-fold, 3-fold to 12-fold, 4-fold to 12-fold, 5-fold to 12-fold, 6-fold to 12-fold, 7-fold to 12-fold, 8-fold to 12-fold, 9-fold to 12-fold, 10-fold to 12-fold, or 11-fold to 12-fold in the inflammatory state relative to the level of ubiquitination when not in the inflammatory state.
Embodiment 129. The method of any one of Embodiments 122 to 128, wherein an elevated level of ubiquitination correlates to an increase in severity of an inflammatory disease state.
Embodiment 130. The method of any one of Embodiments 122 to 129, wherein the pro-inflammatory protein is associated with an inflammatory disease, such as inflammatory bowel disease, Crohn's disease, diverticulitis, and ulcerative colitis.
Embodiment 131. The method of any one of Embodiments 122 to 130, wherein the pro-inflammatory protein is associated with Crohn's disease.
Embodiment 132. The method of any one of Embodiments 122 to 131, wherein the pro-inflammatory protein is associated with ulcerative colitis.

### BRIEF DESCRIPTION OF THE FIGURES

FIGS. 1A-D show the interaction of RIP1-K63 bispecific antibodies with K63 ubiquitin linkage modified RIP1. FIGS. 1A and 1B provide western blot analyses to determine the ability of the indicated antibodies to immunoprecipitate in vitro K63 chain-ubiquitinated and linear chain-ubiquitinated recombinant RIP1 protein. FIGS. 1C and 1D provide western blot analyses of TCL and immunoprecipitated proteins obtained with the indicated antibodies using WT (wild-type, W) and RIP1 KO (knockout) HT29 cells. The indicated antibodies recognize RIP1 modified with K63-linked ubiquitin chains.

FIGS. 2A-D show the interaction of RIP1-ubiquitin chain bispecific or control antibodies in various cell lines, including human colon carcinoma HT29, Fibrosarcoma HT1080, and A549 cells. Specifically, FIGS. 2A-D demonstrate that RIP1-K63 ubiquitin chain bispecific antibodies recognize K63 chain-ubiquitinated RIP1. FIGS. 2A and 2B provide western blot analyses of total cell lysate (TCL) and immunoprecipitated proteins (IP) obtained with the indicated antibodies. The indicated antibodies recognize K63 chain-ubiquitinated RIP1. These results were confirmed by confocal microscopy, shown in FIG. 2C (A549 cells) and FIG. 2D (HT1080 cells).

FIGS. 3A-D show the interaction of RIP1-K63 bispecific antibodies with RIP1 modified with K63-linked ubiquitin chains in various cell lines, including EVSA T, Ku812F, and HT29 cells. FIGS. 3A and 3B provide western blot analyses of TCL and immunoprecipitated proteins obtained with the indicated antibodies. The indicated antibodies recognize RIP1 modified with K63-linked ubiquitin chains. FIGS. 3C and 3D provide western blot analyses of TCL and immunoprecipitated proteins obtained with the indicated antibodies using WT (wild-type, W) and RIP1 KO (knockout) HT29 cells. The indicated antibodies recognize linear chain-ubiquitinated RIP1.

FIGS. 4A-D show the interaction of RIP1-ubiquitin, K63-linear ubiquitin chain or control bispecific antibodies in various cell lines, including human colon carcinoma HT29, Ku812F, and mouse embryonic fibroblasts (MEF) cells. Specifically, FIGS. 4A-D demonstrate that RIP1-Lin ubiquitin chain bispecific antibodies recognize linear chain-ubiquitinated RIP1, whereas RIP1-gD and gD-Lin antibodies, or K63-Lin antibody without stimulation, did not immunoprecipitate ubiquitinated RIP1. FIGS. 4A and 4B provide western blot analyses of TCL and immunoprecipitated proteins obtained with the indicated antibodies. The indicated antibodies recognize linear chain-ubiquitinated RIP1. These results were confirmed by confocal microscopy, shown in FIG. 4C (HT29 cells) and FIG. 4D (MEF cells).

FIGS. 5A-C show the interaction of RIP1-ubiquitin chain, K63-linear ubiquitin chain or control bispecific antibodies in various cell lines, including human colon carcinoma HT29, D645, and THP1 cells. FIGS. 5A, 5B, and 5C provide western blot analyses of TCL and immunoprecipitated proteins obtained with the indicated antibodies. The indicated antibodies recognize RIP1 modified with K63-linked and K63-Lin ubiquitin linked chains.

FIGS. 6A-D show the interaction of RIP1-ubiquitin chain, K63-linear ubiquitin chain or control bispecific antibodies in mouse tissue samples. FIGS. 6A and 6B provide western blot analyses of TCL and immunoprecipitated proteins obtained with the indicated antibodies. The indicated antibodies recognize RIP1 ubiquitinated in vivo with K63-linked and linear ubiquitin chains. FIGS. 6C and 6D provide western blot analyses of immunoprecipitated proteins obtained sith the indicated antibodies and TCL. The indicated antibodies recognize RIP1 ubiquitinated in wild type (WT) or mutant RIP1 (RIP1 K376R knock-in) mouse bone marrow derived macrophages.

FIGS. 7A-E show the interaction of RIP2-ubiquitin chain or control bispecific antibodies in THP1 cells. Specifically, FIGS. 7A-D demonstrate that RIP2-ubiquitin chain bispecific antibodies recognize K63 and linear chain-ubiquitinated RIP2, but single-arm (RIP2-gD, K63-gD, Lin-gD) antibodies do not recognize ubiquitinated RIP2, and even RIP2-K63, RIP2-Lin and K63-Lin bispecific antibodies recognize K63- and linear chain-ubiquitinated RIP2 only after treatment with pathway-relevant stimulus (MDP). FIGS. 7A and 7B provide western blot analyses of TCL and immunoprecipitated proteins obtained with the indicated antibodies. FIG. 7C provides western blot analyses of TCL and immunoprecipitated proteins obtained with the indicated antibodies using WT (wild-type, W) and RIP2 KO (knockout, R2 KO) THP1 cells. The indicated antibodies recognize RIP2 ubiquitinated by linear chains and K63-Lin ubiquitin linked chains. The indicated antibodies recognize K63 and linear chain-ubiquitinated RIP2. FIG. 7D provides western blot analyses of TCL and immunoprecipitated proteins obtained with the indicated antibodies using WT (wild-type, W) and RIP2 KO (knockout, R2 KO) THP1 cells. The indicated antibodies recognize RIP2 modified with K63-linked ubiquitin chains. These results were confirmed by confocal microscopy, shown in FIG. 7E.

FIGS. 8A-G show RIP2-K63 and RIP2-Lin bispecific antibodies as tested in intestinal tissue samples from patients. Specifically, FIGS. 8A-F demonstrate that RIP2-K63 and RIP2-Lin bispecific antibodies can be used to investigate ubiquitination status of RIP2 in IBD samples. FIGS. 8A and 8B provide western blot analyses to determine the ability of the indicated antibodies to detect immunoprecipitated proteins from samples collected from patients with intestinal cancer, dysplasia, diverticulitis (DIV), Crohn's disease (CD) or ulcerative colitis (UC). FIGS. 8C and 8D show Samples 1-52 and Samples 53-92, respectively, from patients of FIG. 8A. FIG. 8E provides expression of RIP2 in patient samples. FIG. 8F provides quantification of RIP2 ubiquitination by scanning western blots following immunoprecitation with the antibodies indicated in FIGS. 8C and 8D. FIG. 8G shows the data of FIG. 8F in bar graph format.

FIGS. 9A-E show the mechanism by which K63-Lin bispecific antibody can be used to detect and identify proteins ubiquitinated with K63-linked and linear chains in different signaling pathways. FIG. 9A provides a schematic of the experimental design scheme, wherein THP1 cells were treated with vehicle (phosphate buffered saline, PBS), tumor necrosis factor (TNF), muramyl dipeptide (MDP), or lipopolysaccharide (LPS) for the indicated time periods, followed by lysis and immunoprecipitation with the indicated bispecific antibody and mass spectroscopy (MS) analysis, resulting in identification of the proteins in FIG. 9B, as also determined in the western blot analyses of FIG. 9C. Ubiquitinated RIP2 was identified from the MDP-treated condition by tandem mass spectrometry (ms/ms, FIG. 9D) and extracted ion chromatography (XIC), with matched peaks highlighted by arrows (FIG. 9E).

FIG. 10A-E shows identification of TRADD (FIG. 10A) (SEQ ID NO: 104), TNFR1 (FIG. 10B) (SEQ ID NO: 105), RIP1 (FIG. 10C) (SEQ ID NO: 106), NOD2 (FIG. 10D) (SEQ ID NO: 107) and IRAK1 (FIG. 10E) (SEQ ID NO: 108) from the conditions indicated in FIGS. 9A and 9B. Representative ms/ms spectra for each protein are annotated.

### DETAILED DESCRIPTION OF CERTAIN EMBODIMENTS

### I. Definitions

As used herein, VH refers to a heavy chain variable domain and VL refers to a light chain variable domain.

An “acceptor human framework” for the purposes herein is a framework comprising the amino acid sequence of a VL framework or a VH framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

As used herein, “about” refers to a value that is 10% more or less than a stated value, gives results functionally equivalent to the stated value, or rounds to the stated value.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

“Avidity” refers to the strength of the sum total of noncovalent interactions between a molecule (e.g., an antibody) and its binding partner (e.g., a target molecule comprising one or more antigens). The avidity of a molecule X for its partner Y can generally be represented by the dissociation constant (Kd). A bispecific antibody will generally have a greater avidity for a binding partner comprising epitopes recognized by both of the antigen binding sites of the bispecific antibody than for a binding partner comprising either of the eptiopes individually. Avidity can be measured by common methods known in the art, including those described herein. Specific illustrative and exemplary embodiments for measuring binding avidity are described in the following. The term “functional affinity” is sometimes used in the art to refer to avidity.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term “multispecific antibody” as used herein refers to an antibody comprising an antigen-binding domain that has polyepitopic specificity (i.e., is capable of binding to two, or more, different epitopes on one molecule or is capable of binding to epitopes on two, or more, different molecules).

An “agonist antibody” as used herein is an antibody which mimics at least one of the functional activities of a polypeptide of interest.

An “antagonist antibody” or a “blocking antibody” is an antibody which inhibits or reduces biological activity of the antigen to which it specifically binds. Certain blocking antibodies or antagonist antibodies substantially or completely inhibit the biological activity of the antigen.

The term “antibody drug conjugate” (ADC) as used herein is equivalent to the term “immunoconjugate”.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

As used herein, the term “anti-polyubiquitin antibody” refers to an antibody that is capable of specifically binding to a polyubiquitin molecule. In certain embodiments, an antibody that binds to a polyubiquitin has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M).

As used herein, the terms “anti-ubiquitin antibody” and “anti-monoubiquitin antibody” are used interchangeably, and refer to an antibody that is capable of specifically binding to a ubiquitin molecule.

As used herein, the term “anti-RIP1 antibody” refers to an antibody that is capable of specifically binding to a receptor-interacting protein kinase 1 (“receptor-interacting protein-1 kinase,” “RIP1” or “RIPK1”) molecule. In certain embodiments, an antibody that binds to a RIP1 has a dissociation constant (Kd) of ≤1 μM, ≤100 nM, ≤10 nM, ≤1 nM, ≤0.1 nM, ≤0.01 nM, or ≤0.001 nM (e.g. 10−8M or less, e.g. from 10⁻⁸ M to 10⁻¹³ M, e.g., from 10⁻⁹ M to 10⁻¹³ M, e.g., from 1-20 nM, such as 1-15 nM, e.g., 1-12 nM, e.g., 1-10 nM).

As used herein, the term “anti-RIP2 antibody” refers to an antibody that is capable of specifically binding to a receptor-interacting protein kinase 2 (“receptor-interacting serine/threonine-protein kinase 2,” “RIP2” or “RIPK2”) molecule. In certain embodiments, an antibody that binds to a RIP2 has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M).

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” refers to the particular site on an antigen molecule to which an antibody binds.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

As used herein, “first,” “second,” etc. are used with reference to elements of a complex structure, e.g., a protein with tertiary/quaternary structure such as an antibody, to refer to those elements (e.g., monomers, chains, domains) without any implication as to the ordering or positioning of the elements; thus a “first” element may be C- or N-terminal to a second element, or closer or farther from one end or another of the structure than a second element. Thus, for example, with reference to the halves of a bispecific antibody, the designation of a half or a linkage as first or second is arbitrary.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The term “half antibody” is used herein to refer to one arm of an antibody and includes at least a VH domain and one CH domain.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “rabbit antibody” is one which possesses an amino acid sequence which corresponds to that of an antibody produced by a rabbit or a rabbit cell or derived from a non-rabbit source that utilizes rabbit antibody repertoires or other rabbit antibody-encoding sequences.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, such as one or two variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent. An immunoconjugate is equivalent to the term “antibody drug conjugate” (ADC).

An “individual” or “patient” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “inflammatory disease” includes Crohn's disease, diverticulitis, graft versus host disease (GVHD), inflammatory bowel disease, kidney injury and delated graft function, multiple sclerosis, rheumatoid arthritis, skin inflammatory diseases, stroke and ulcerative colitis.

An “isolated antibody” is one which has been separated from a component of its natural environment. In some embodiments, an antibody is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC). For a review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (x) and lambda (k), based on the amino acid sequence of its constant domain.

“Or” is used in the inclusive sense, i.e., equivalent to “and/or,” unless the context requires otherwise.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “selectively recognizes” means that the referenced antibody does not bind proteins of other than the polyubiquitinated protein of interest or binds other proteins with substantially weaker affinity (e.g., 50-fold, 100-fold, 200-fold, 500-fold, or 1000-fold weaker affinity).

As used herein, “substantially free of” means that the referenced entity is absent or, if present, is (i) in a sufficiently low quantity so as not to significantly alter a functional property or result of a composition, method, use, or step, as the case may be; (ii) is undetectable by at least one appropriate analytical method, such as mass spectrometry (e.g., MALDI-TOF or any MS procedure used in the Examples), blotting (e.g., Western for a polypeptide), or electrophoresis (e.g., SDS-PAGE with Coomassie blue or silver staining); or (iii) is present in an amount less than or equal to about 20%, 15%, 10%, 5%, 4%, 3%, 2%, 1%, 0.5%, or 0.1%, by mass or by mole fraction relative to the total amount of non-solvent material in the composition.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies disclosed herein are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In some aspects, antibodies that bind to polyubiqutinated proteins, such as polyubiquitinated proinflammatory proteins are provided. Such antibodies are useful, e.g., for detecting, modulating the activity of, or immunoprecipitating polyubiquitinated proteins.

### A. Exemplary Antibodies

In some embodiments, a multispecific antibody comprises a first half antibody comprising a first antigen binding site that binds to a polyubiquitin and a second half antibody comprising a second antigen binding site that binds receptor-interacting protein kinase 1 (RIP1). In some embodiments, an antibody selectively recognizes polyubiqinated RIP1. In some embodiments, the antibody does not recognize receptor-interacting protein kinase 2 (RIP2) and/or does not recognize non-ubiquitinated RIP1.

In some embodiments, a multispecific antibody comprises a first half antibody comprising a first antigen binding site that binds to a polyubiquitin and a second half antibody comprising a second antigen binding site that binds receptor-interacting protein kinase 2 (RIP2). In some embodiments, an antibody selectively recognizes polyubiqinated RIP2. In some embodiments, the antibody does not recognize receptor-interacting protein kinase 1 (RIP1) and/or does not recognize non-ubiquitinated RIP2.

In some embodiments, the polyubiquitin comprises a K11, K48, K63, or M1 (C-terminal to N-terminal)-linkage. In some embodiments, the polyubiquitin has homogenous topology.

The antibodies, antibody sequences, and sequence listing disclosed in U.S. Pat. No. 7,763,245 are incorporated herein by reference. In some embodiments, the first half antibody comprising a first antigen binding site that binds to a polyubiquitin comprises the HVR-H1, HVR-H2, HVR-H3, HVR-L1, HVR-L2, and HVR-L3 of an antibody disclosed in U.S. Pat. No. 7,763,245 that binds a polyubiquitin. In some embodiments, the first half antibody comprising a first antigen binding site that binds to a polyubiquitin comprises a combination of HVR-H1, HVR-H2, HVR-H3, HVR-L1, HVR-L2, and HVR-L3 selected from the HVRs disclosed in U.S. Pat. No. 7,763,245, wherein the half antibody binds a polyubiquitin.

The antibodies, antibody sequences, and sequence listing disclosed in U.S. Pat. No. 8,133,488 are incorporated herein by reference. In some embodiments, the first half antibody comprising a first antigen binding site that binds to a polyubiquitin comprises the HVR-H1, HVR-H2, HVR-H3, HVR-L1, HVR-L2, and HVR-L3 of an antibody disclosed in U.S. Pat. No. 8,133,488 that binds a polyubiquitin. In some embodiments, the first half antibody comprising a first antigen binding site that binds to a polyubiquitin comprises a combination of HVR-H1, HVR-H2, HVR-H3, HVR-L1, HVR-L2, and HVR-L3 selected from the HVRs disclosed in U.S. Pat. No. 8,133,488, wherein the half antibody binds a polyubiquitin.

The antibodies, antibody sequences, and sequence listing disclosed in U.S. Pat. No. 8,992,919 are incorporated herein by reference. In some embodiments, the first half antibody comprising a first antigen binding site that binds to a polyubiquitin comprises the HVR-H1, HVR-H2, HVR-H3, HVR-L1, HVR-L2, and HVR-L3 of an antibody disclosed in U.S. Pat. No. 8,992,919 that binds a polyubiquitin. In some embodiments, the first half antibody comprising a first antigen binding site that binds to a polyubiquitin comprises a combination of HVR-H1, HVR-H2, HVR-H3, HVR-L1, HVR-L2, and HVR-L3 selected from the HVRs disclosed in U.S. Pat. No. 8,992,919, wherein the half antibody binds a polyubiquitin.

The antibodies, antibody sequences, and sequence listing disclosed in U.S. Pat. No. 9,321,844 are incorporated herein by reference. In some embodiments, the first half antibody comprising a first antigen binding site that binds to a polyubiquitin comprises the HVR-H1, HVR-H2, HVR-H3, HVR-L1, HVR-L2, and HVR-L3 of an antibody disclosed in U.S. Pat. No. 9,321,844 that binds a polyubiquitin. In some embodiments, the first half antibody comprising a first antigen binding site that binds to a polyubiquitin comprises a combination of HVR-H1, HVR-H2, HVR-H3, HVR-L1, HVR-L2, and HVR-L3 selected from the HVRs disclosed in U.S. Pat. No. 9,321,844, wherein the half antibody binds a polyubiquitin.

In some embodiments, the first half antibody comprising a first antigen binding site that binds to a polyubiquitin comprises an HVR-L1, HVR-L2, HVR-L3, HVR-H1, HVR-H2, and HVR-H3 comprising the amino acid sequences of SEQ ID NOs: 9, 10, 11, 12, 13, and 14, respectively. In some embodiments, the first half antibody comprising a first antigen binding site that binds to a polyubiquitin comprises an HVR-L1, HVR-L2, HVR-L3, HVR-H1, HVR-H2, and HVR-H3 comprising the amino acid sequences of SEQ ID NOs: 23, 24, 25, 26, 27, and 28, respectively. In some embodiments, the first half antibody comprising a first antigen binding site that binds to a polyubiquitin comprises an HVR-L1, HVR-L2, HVR-L3, HVR-H1, HVR-H2, and HVR-H3 comprising the amino acid sequences of SEQ ID NOs: 37, 38, 39, 40, 41, and 42, respectively. In some embodiments, the first half antibody comprising a first antigen binding site that binds to a polyubiquitin comprises an HVR-L1, HVR-L2, HVR-L3, HVR-H1, HVR-H2, and HVR-H3 comprising the amino acid sequences of SEQ ID NOs: 51, 52, 53, 54, 55, and 56, respectively.

In some embodiments, the second half antibody comprises a second antigen binding site that binds RIP1 and comprises an HVR-L1, HVR-L2, HVR-L3, HVR-H1, HVR-H2, and HVR-H3 comprising the amino acid sequences of SEQ ID NO: 74, SEQ ID NO: 75, SEQ ID NO: 76, SEQ ID NO: 78, SEQ ID NO: 79, and SEQ ID NO: 80, respectively. In some embodiments, the second half antibody comprises a second antigen binding site that binds RIP1 and comprises an HVR-L1, HVR-L2, HVR-L3, HVR-H1, HVR-H2, and HVR-H3 comprising the amino acid sequences of SEQ ID NO: 74, SEQ ID NO: 77, SEQ ID NO: 76, SEQ ID NO: 78, SEQ ID NO: 79, and SEQ ID NO: 80, respectively.

In some embodiments, the second half antibody comprises a second antigen binding site that binds RIP2 and comprises an HVR-L1, HVR-L2, HVR-L3, HVR-H1, HVR-H2, and HVR-H3 comprising the amino acid sequences of SEQ ID NO: 95, SEQ ID NO: 96, SEQ ID NO: 97, SEQ ID NO: 98, SEQ ID NO: 99, and SEQ ID NO: 100, respectively.

In some embodiments, the first half antibody comprises
(i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14, and
the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14, and
the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14, and the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100.

In some embodiments, the first half antibody comprises
(i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28, and the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28, and
the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28, and
the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100.

In some embodiments, the first half antibody comprises
(i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42, and
the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42, and
the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42, and
the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100.

In some embodiments the first half antibody comprises
(i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56, and
the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56, and
the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80.

In some embodiments, the first half antibody comprises
(i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56, and
the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100.

In some embodiments, the first half antibody comprises a VL sequence with at least about 90%, 91%, 92%, 93%, 94%, 95%, 96%, 97%, 98%, 99%, or 100% sequence identity to SEQ ID NO: 7 and a VH sequence with at least about 90%, 91%, 92%, 93%, 94%, 95%, 96%, 97%, 98%, 99%, or 100% sequence identity to SEQ ID NO: 8. In some embodiments, the first half antibody comprises a VL sequence with at least about 90%, 91%, 92%, 93%, 94%, 95%, 96%, 97%, 98%, 99%, or 100% sequence identity to SEQ ID NO: 21 and a VH sequence with at least about 90%, 91%, 92%, 93%, 94%, 95%, 96%, 97%, 98%, 99%, or 100% sequence identity to SEQ ID NO: 22. In some embodiments, the first half antibody comprises a VL sequence with at least about 90%, 91%, 92%, 93%, 94%, 95%, 96%, 97%, 98%, 99%, or 100% sequence identity to SEQ ID NO: 35 and a VH sequence with at least about 90%, 91%, 92%, 93%, 94%, 95%, 96%, 97%, 98%, 99%, or 100% sequence identity to SEQ ID NO: 36. In some embodiments, the first half antibody comprises a VL sequence with at least about 90%, 91%, 92%, 93%, 94%, 95%, 96%, 97%, 98%, 99%, or 100% sequence identity to SEQ ID NO: 49 and a VH sequence with at least about 90%, 91%, 92%, 93%, 94%, 95%, 96%, 97%, 98%, 99%, or 100% sequence identity to SEQ ID NO: 50.

In some embodiments, the first half antibody comprises VH and VL sequences at least about 90%, 91%, 92%, 93%, 94%, 95%, 96%, 97%, 98%, 99%, or 100% identical to the VH and VL sequences of an antibody disclosed in U.S. Pat. No. 7,763,245 that binds a polyubiquitin. In some embodiments, the first half antibody comprises a combination of VH and VL sequences disclosed in U.S. Pat. No. 7,763,245, wherein the half antibody binds a polyubiquitin.

In some embodiments, the first half antibody comprises VH and VL sequences at least about 90%, 91%, 92%, 93%, 94%, 95%, 96%, 97%, 98%, 99%, or 100% identical to the VH and VL sequences of an antibody disclosed in U.S. Pat. No. 8,133,488 that binds a polyubiquitin. In some embodiments, the first half antibody comprises a combination of VH and VL sequences disclosed in U.S. Pat. No. 8,133,488, wherein the half antibody binds a polyubiquitin.

In some embodiments, the first half antibody comprises VH and VL sequences at least about 90%, 91%, 92%, 93%, 94%, 95%, 96%, 97%, 98%, 99%, or 100% identical to the VH and VL sequences of an antibody disclosed in U.S. Pat. No. 8,992,919 that binds a polyubiquitin. In some embodiments, the first half antibody comprises a combination of VH and VL sequences disclosed in U.S. Pat. No. 8,992,919, wherein the half antibody binds a polyubiquitin.

In some embodiments, the first half antibody comprises VH and VL sequences at least about 90%, 91%, 92%, 93%, 94%, 95%, 96%, 97%, 98%, 99%, or 100% identical to the VH and VL sequences of an antibody disclosed in U.S. Pat. No. 9,321,844 that binds a polyubiquitin. In some embodiments, the first half antibody comprises a combination of VH and VL sequences disclosed in U.S. Pat. No. 9,321,844, wherein the half antibody binds a polyubiquitin.

In some embodiments, the first half antibody comprises the VH and VL sequences of an antibody disclosed in U.S. Pat. No. 7,763,245 that binds a polyubiquitin. In some embodiments, the first half antibody comprises a combination of VH and VL sequences disclosed in U.S. Pat. No. 7,763,245, wherein the half antibody binds a polyubiquitin.

In some embodiments, the first half antibody comprises the VH and VL sequences of an antibody disclosed in U.S. Pat. No. 8,133,488 that binds a polyubiquitin. In some embodiments, the first half antibody comprises a combination of VH and VL sequences disclosed in U.S. Pat. No. 8,133,488, wherein the half antibody binds a polyubiquitin.

In some embodiments, the first half antibody comprises the VH and VL sequences of an antibody disclosed in U.S. Pat. No. 8,992,919 that binds a polyubiquitin. In some embodiments, the first half antibody comprises a combination of VH and VL sequences disclosed in U.S. Pat. No. 8,992,919, wherein the half antibody binds a polyubiquitin.

In some embodiments, the first half antibody comprises the VH and VL sequences of an antibody disclosed in U.S. Pat. No. 9,321,844 that binds a polyubiquitin. In some embodiments, the first half antibody comprises a combination of VH and VL sequences disclosed in U.S. Pat. No. 9,321,844, wherein the half antibody binds a polyubiquitin.

In some embodiments, the second half antibody comprises a VL sequence with at least about 90%, 91%, 92%, 93%, 94%, 95%, 96%, 97%, 98%, 99%, or 100% sequence identity to SEQ ID NO: 71 and a VH sequence with at least about 90%, 91%, 92%, 93%, 94%, 95%, 96%, 97%, 98%, 99%, or 100% sequence identity to SEQ ID NO: 73. In some embodiments, the second half antibody comprises a VL sequence with at least about 90%, 91%, 92%, 93%, 94%, 95%, 96%, 97%, 98%, 99%, or 100% sequence identity to SEQ ID NO: 72 and a VH sequence with at least about 90%, 91%, 92%, 93%, 94%, 95%, 96%, 97%, 98%, 99%, or 100% sequence identity to SEQ ID NO: 73.

In some embodiments, the second half antibody comprises a VL sequence with at least about 90%, 91%, 92%, 93%, 94%, 95%, 96%, 97%, 98%, 99%, or 100% sequence identity to SEQ ID NO: 93 and a VH sequence with at least about 90%, 91%, 92%, 93%, 94%, 95%, 96%, 97%, 98%, 99%, or 100% sequence identity to SEQ ID NO: 94.

In some embodiments, a) the first half antibody comprises a VL sequence with at least about 90%, 91%, 92%, 93%, 94%, 95%, 96%, 97%, 98%, 99%, or 100% sequence identity to SEQ ID NO: 7 and a VH sequence with at least about 90%, 91%, 92%, 93%, 94%, 95%, 96%, 97%, 98%, 99%, or 100% sequence identity to SEQ ID NO: 8, and the second half antibody comprises a VL sequence with at least about 90%, 91%, 92%, 93%, 94%, 95%, 96%, 97%, 98%, 99%, or 100% sequence identity to SEQ ID NO: 71 and a VH sequence with at least about 90%, 91%, 92%, 93%, 94%, 95%, 96%, 97%, 98%, 99%, or 100% sequence identity to SEQ ID NO: 73. In some embodiments, the first half antibodies comprises a VL sequence with at least about 90%, 91%, 92%, 93%, 94%, 95%, 96%, 97%, 98%, 99%, or 100% sequence identity to SEQ ID NO: 7 and a VH sequence with at least about 90%, 91%, 92%, 93%, 94%, 95%, 96%, 97%, 98%, 99%, or 100% sequence identity to SEQ ID NO: 8, and the second half antibody comprises a VL sequence with at least about 90%, 91%, 92%, 93%, 94%, 95%, 96%, 97%, 98%, 99%, or 100% sequence identity to SEQ ID NO: 72 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 7 and a VH sequence with at least about 90%, 91%, 92%, 93%, 94%, 95%, 96%, 97%, 98%, 99%, or 100% sequence identity to SEQ ID NO: 8, and the second half antibody comprises a VL sequence with at least about 90%, 91%, 92%, 93%, 94%, 95%, 96%, 97%, 98%, 99%, or 100% sequence identity to SEQ ID NO: 93 and a VH sequence with at least about 90%, 91%, 92%, 93%, 94%, 95%, 96%, 97%, 98%, 99%, or 100% sequence identity to SEQ ID NO: 94.

In some embodiments, the first half antibody comprises a VL sequence with at least about 90%, 91%, 92%, 93%, 94%, 95%, 96%, 97%, 98%, 99%, or 100% sequence identity to SEQ ID NO: 21 and a VH sequence with at least about 90%, 91%, 92%, 93%, 94%, 95%, 96%, 97%, 98%, 99%, or 100% sequence identity to SEQ ID NO: 22, and the second half antibody comprises a VL sequence with at least about 90%, 91%, 92%, 93%, 94%, 95%, 96%, 97%, 98%, 99%, or 100% sequence identity to SEQ ID NO: 71 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 21 and a VH sequence with at least about 90%, 91%, 92%, 93%, 94%, 95%, 96%, 97%, 98%, 99%, or 100% sequence identity to SEQ ID NO: 22, and the second half antibody comprises a VL sequence with at least about 90%, 91%, 92%, 93%, 94%, 95%, 96%, 97%, 98%, 99%, or 100% sequence identity to SEQ ID NO: 72 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 21 and a VH sequence with at least about 90%, 91%, 92%, 93%, 94%, 95%, 96%, 97%, 98%, 99%, or 100% sequence identity to SEQ ID NO: 22, and the second half antibody comprises a VL sequence with at least about 90%, 91%, 92%, 93%, 94%, 95%, 96%, 97%, 98%, 99%, or 100% sequence identity to SEQ ID NO: 93 and a VH sequence with at least about 90%, 91%, 92%, 93%, 94%, 95%, 96%, 97%, 98%, 99%, or 100% sequence identity to SEQ ID NO: 94.

In some embodiments, the first half antibody comprises a VL sequence with at least about 90%, 91%, 92%, 93%, 94%, 95%, 96%, 97%, 98%, 99%, or 100% sequence identity to SEQ ID NO: 35 and a VH sequence with at least about 90%, 91%, 92%, 93%, 94%, 95%, 96%, 97%, 98%, 99%, or 100% sequence identity to SEQ ID NO: 36, and the second half antibody comprises a VL sequence with at least about 90%, 91%, 92%, 93%, 94%, 95%, 96%, 97%, 98%, 99%, or 100% sequence identity to SEQ ID NO: 71 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 35 and a VH sequence with at least about 90%, 91%, 92%, 93%, 94%, 95%, 96%, 97%, 98%, 99%, or 100% sequence identity to SEQ ID NO: 36, and the second half antibody comprises a VL sequence with at least about 90%, 91%, 92%, 93%, 94%, 95%, 96%, 97%, 98%, 99%, or 100% sequence identity to SEQ ID NO: 72 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 35 and a VH sequence with at least about 90%, 91%, 92%, 93%, 94%, 95%, 96%, 97%, 98%, 99%, or 100% sequence identity to SEQ ID NO: 36, and the second half antibody comprises a VL sequence with at least about 90%, 91%, 92%, 93%, 94%, 95%, 96%, 97%, 98%, 99%, or 100% sequence identity to SEQ ID NO: 93 and a VH sequence with at least about 90%, 91%, 92%, 93%, 94%, 95%, 96%, 97%, 98%, 99%, or 100% sequence identity to SEQ ID NO: 94.

In some embodiments, the first half antibody comprises a VL sequence with at least about 90%, 91%, 92%, 93%, 94%, 95%, 96%, 97%, 98%, 99%, or 100% sequence identity to SEQ ID NO: 49 and a VH sequence with at least about 90%, 91%, 92%, 93%, 94%, 95%, 96%, 97%, 98%, 99%, or 100% sequence identity to SEQ ID NO: 50, and the second half antibody comprises a VL sequence with at least about 90%, 91%, 92%, 93%, 94%, 95%, 96%, 97%, 98%, 99%, or 100% sequence identity to SEQ ID NO: 71 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 49 and a VH sequence with at least about 90%, 91%, 92%, 93%, 94%, 95%, 96%, 97%, 98%, 99%, or 100% sequence identity to SEQ ID NO: 50, and the second half antibody comprises a VL sequence with at least about 90%, 91%, 92%, 93%, 94%, 95%, 96%, 97%, 98%, 99%, or 100% sequence identity to SEQ ID NO: 72 and a VH sequence with at least about 90%, 91%, 92%, 93%, 94%, 95%, 96%, 97%, 98%, 99%, or 100% sequence identity to SEQ ID NO: 73. In some embodiments, the first half antibody comprises a VL sequence with at least about 90%, 91%, 92%, 93%, 94%, 95%, 96%, 97%, 98%, 99%, or 100% sequence identity to SEQ ID NO: 49 and a VH sequence with at least about 90%, 91%, 92%, 93%, 94%, 95%, 96%, 97%, 98%, 99%, or 100% sequence identity to SEQ ID NO: 50, and the second half antibody comprises a VL sequence with at least about 90%, 91%, 92%, 93%, 94%, 95%, 96%, 97%, 98%, 99%, or 100% sequence identity to SEQ ID NO: 93 and a VH sequence with at least about 90%, 91%, 92%, 93%, 94%, 95%, 96%, 97%, 98%, 99%, or 100% sequence identity to SEQ ID NO: 94.

In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody comprising that sequence retains the ability to bind to a polyubiquitin. In certain embodiments, a total of 1 to 10 amino acids have been substituted, inserted and/or deleted in a VH sequence. In certain embodiments, substitutions, insertions, or deletions occur in regions outside the HVRs (i.e., in the FRs). Optionally, the antibody comprises a VH sequence discussed above, including post-translational modifications of that sequence.

In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody comprising that sequence retains the ability to bind to a polyubiquitin. In certain embodiments, a total of 1 to 10 amino acids have been substituted, inserted and/or deleted in a VL sequence. In certain embodiments, substitutions, insertions, or deletions occur in regions outside the HVRs (i.e., in the FRs). Optionally, the antibody comprises a VL sequence discussed above, including post-translational modifications of that sequence.

In some embodiments, the antibody is humanized. In some embodiments, the antibody comprises HVRs as in any of the above embodiments, and further comprises a human acceptor framework, e.g. a human immunoglobulin framework or a human consensus framework. In some embodiments, the antibody comprises HVRs as in any of the above embodiments and rabbit framework regions.

In some aspects, an antibody that binds to the same epitopes as a bispecific antibody provided herein is provided. For example, in certain embodiments, an antibody is provided that binds to the same epitopes as an antibody comprising first and second half antibodies, wherein:
a) the first half antibody comprises a VL sequence of SEQ ID NO: 7 and a VH sequence of SEQ ID NO: 8,
and the second half antibody comprises a VL sequence of SEQ ID NO: 71 and a VH sequence of SEQ ID NO: 73;
b) the first half antibody comprises a VL sequence of SEQ ID NO: 7 and a VH sequence of SEQ ID NO: 8,
and the second half antibody comprises a VL sequence of SEQ ID NO: 72 and a VH sequence of SEQ ID NO: 73;
c) the first half antibody comprises a VL sequence of SEQ ID NO: 7 and a VH sequence of SEQ ID NO: 8,
and the second half antibody comprises a VL sequence of SEQ ID NO: 93 and a VH sequence of SEQ ID NO: 94;
d) the first half antibody comprises a VL sequence of SEQ ID NO: 21 and a VH sequence of SEQ ID NO: 22,
and the second half antibody comprises a VL sequence of SEQ ID NO: 71 and a VH sequence of SEQ ID NO: 73;
e) the first half antibody comprises a VL sequence of SEQ ID NO: 21 and a VH sequence of SEQ ID NO: 22,
and the second half antibody comprises a VL sequence of SEQ ID NO: 72 and a VH sequence of SEQ ID NO: 73;
f) the first half antibody comprises a VL sequence of SEQ ID NO: 21 and a VH sequence of SEQ ID NO: 22,
and the second half antibody comprises a VL sequence of SEQ ID NO: 93 and a VH sequence of SEQ ID NO: 94;
g) the first half antibody comprises a VL sequence of SEQ ID NO: 35 and a VH sequence of SEQ ID NO: 36,
and the second half antibody comprises a VL sequence of SEQ ID NO: 71 and a VH sequence of SEQ ID NO: 73;
h) the first half antibody comprises a VL sequence of SEQ ID NO: 35 and a VH sequence of SEQ ID NO: 36,
and the second half antibody comprises a VL sequence of SEQ ID NO: 72 and a VH sequence of SEQ ID NO: 73;
i) the first half antibody comprises a VL sequence of SEQ ID NO: 35 and a VH sequence of SEQ ID NO: 36,
and the second half antibody comprises a VL sequence of SEQ ID NO: 93 and a VH sequence of SEQ ID NO: 94;
j) the first half antibody comprises a VL sequence of SEQ ID NO: 49 and a VH sequence of SEQ ID NO: 50,
and the second half antibody comprises a VL sequence of SEQ ID NO: 71 and a VH sequence of SEQ ID NO: 73;
1) the first half antibody comprises a VL sequence of SEQ ID NO: 49 and a VH sequence of SEQ ID NO: 50,
and the second half antibody comprises a VL sequence of SEQ ID NO: 72 and a VH sequence of SEQ ID NO: 73; or
1) the first half antibody comprises a VL sequence of SEQ ID NO: 49 and a VH sequence of SEQ ID NO: 50,
and the second half antibody comprises a VL sequence of SEQ ID NO: 93 and a VH sequence of SEQ ID NO: 94.

In some embodiments, a bispecific antibody comprising VH and VL sequences as in one of a) through 1) in the preceding paragraph is provided.

In some embodiments, the antibody is a monoclonal antibody, including a chimeric, humanized or human antibody. In some embodiments, the antibody is an antibody fragment, e.g., a dimeric scFv, diabody, or F(ab′)2 fragment. In another embodiment, the antibody is a substantially full length antibody, e.g., an IgG1, IgG2a, IgG2b, IgG3, or IgG4 antibody, or other antibody class or isotype as defined herein.

In some embodiments, the antibody comprises at least one heavy chain with a C-terminal lysine. In some embodiments, the antibody comprises at least one heavy chain lacking a C-terminal lysine. In some embodiments, the antibody comprises only heavy chains without a C-terminal lysine. C-terminal lysines can be removed, e.g., enzymatically, such as by carboxypeptidase treatment, or genetically, such as by deletion or substitution of the lysine codon at the 3′ end of a heavy chain coding sequence. Heavy chain C-terminal lysines are located far from antigen binding sites and dispensable for binding activity, and their removal can provide more homogeneous antibody preparations.

In some embodiments, the first half antibody comprises
a) a light chain sequence having at least about 90%, 91%, 92%, 93%, 94%, 95%, 96%, 97%, 98%, 99%, or 100% sequence identity to SEQ ID NO: 2 and a heavy chain sequence having at least about 90%, 91%, 92%, 93%, 94%, 95%, 96%, 97%, 98%, 99%, or 100% sequence identity to SEQ ID NO: 4;
b) a light chain sequence having at least about 90%, 91%, 92%, 93%, 94%, 95%, 96%, 97%, 98%, 99%, or 100% sequence identity to SEQ ID NO: 16 and a heavy chain sequence having at least about 90%, 91%, 92%, 93%, 94%, 95%, 96%, 97%, 98%, 99%, or 100% sequence identity to SEQ ID NO: 18;
c) a light chain sequence having at least about 90%, 91%, 92%, 93%, 94%, 95%, 96%, 97%, 98%, 99%, or 100% sequence identity to SEQ ID NO: 30 and a heavy chain sequence having at least about 90%, 91%, 92%, 93%, 94%, 95%, 96%, 97%, 98%, 99%, or 100% sequence identity to SEQ ID NO: 32; or
d) a light chain sequence having at least about 90%, 91%, 92%, 93%, 94%, 95%, 96%, 97%, 98%, 99%, or 100% sequence identity to SEQ ID NO: 44 and a heavy chain sequence having at least about 90%, 91%, 92%, 93%, 94%, 95%, 96%, 97%, 98%, 99%, or 100% sequence identity to SEQ ID NO: 46,
optionally wherein a C-terminal lysine is missing from one or more heavy chains.

In some embodiments, the first half antibody comprises
a) a light chain sequence having at least about 90%, 91%, 92%, 93%, 94%, 95%, 96%, 97%, 98%, 99%, or 100% sequence identity to SEQ ID NO: 2 and a heavy chain sequence having at least about 90%, 91%, 92%, 93%, 94%, 95%, 96%, 97%, 98%, 99%, or 100% sequence identity to SEQ ID NO: 6;
b) a light chain sequence having at least about 90%, 91%, 92%, 93%, 94%, 95%, 96%, 97%, 98%, 99%, or 100% sequence identity to SEQ ID NO: 16 and a heavy chain sequence having at least about 90%, 91%, 92%, 93%, 94%, 95%, 96%, 97%, 98%, 99%, or 100% sequence identity to SEQ ID NO: 20;
c) a light chain sequence having at least about 90%, 91%, 92%, 93%, 94%, 95%, 96%, 97%, 98%, 99%, or 100% sequence identity to SEQ ID NO: 30 and a heavy chain sequence having at least about 90%, 91%, 92%, 93%, 94%, 95%, 96%, 97%, 98%, 99%, or 100% sequence identity to SEQ ID NO: 34; or
d) a light chain sequence having at least about 90%, 91%, 92%, 93%, 94%, 95%, 96%, 97%, 98%, 99%, or 100% sequence identity to SEQ ID NO: 44 and a heavy chain sequence having at least about 90%, 91%, 92%, 93%, 94%, 95%, 96%, 97%, 98%, 99%, or 100% sequence identity to SEQ ID NO: 48, optionally wherein a C-terminal lysine is missing from one or more chains.

In some embodiments, the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 68; optionally wherein a C-terminal lysine is missing from one or more heavy chains.

In some embodiments, the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 70; optionally wherein a C-terminal lysine is missing from one or more heavy chains.

In some embodiments, the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 90; optionally wherein a C-terminal lysine is missing from one or more heavy chains.

In some embodiments, the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 92; optionally wherein a C-terminal lysine is missing from one or more heavy chains.

In some embodiments, the antibody comprises first and second half antibodies, wherein
a) the first half antibody comprises a light chain sequence having at least about 90%, 91%, 92%, 93%, 94%, 95%, 96%, 97%, 98%, 99%, or 100% sequence identity to SEQ ID NO: 2 and a heavy chain sequence having at least about 90%, 91%, 92%, 93%, 94%, 95%, 96%, 97%, 98%, 99%, or 100% sequence identity to SEQ ID NO: 4, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 70;
b) the first half antibody comprises a light chain sequence having at least about 90%, 91%, 92%, 93%, 94%, 95%, 96%, 97%, 98%, 99%, or 100% sequence identity to SEQ ID NO: 2 and a heavy chain sequence having at least about 90%, 91%, 92%, 93%, 94%, 95%, 96%, 97%, 98%, 99%, or 100% sequence identity to SEQ ID NO: 6, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 68;
c) the first half antibody comprises a light chain sequence having at least about 90%, 91%, 92%, 93%, 94%, 95%, 96%, 97%, 98%, 99%, or 100% sequence identity to SEQ ID NO: 16 and a heavy chain sequence having at least about 90%, 91%, 92%, 93%, 94%, 95%, 96%, 97%, 98%, 99%, or 100% sequence identity to SEQ ID NO: 18, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 70;
d) the first half antibody comprises a light chain sequence having at least about 90%, 91%, 92%, 93%, 94%, 95%, 96%, 97%, 98%, 99%, or 100% sequence identity to SEQ ID NO: 16 and a heavy chain sequence having at least about 90%, 91%, 92%, 93%, 94%, 95%, 96%, 97%, 98%, 99%, or 100% sequence identity to SEQ ID NO: 20, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 68;
e) the first half antibody comprises a light chain sequence having at least about 90%, 91%, 92%, 93%, 94%, 95%, 96%, 97%, 98%, 99%, or 100% sequence identity to SEQ ID NO: 30 and a heavy chain sequence having at least about 90%, 91%, 92%, 93%, 94%, 95%, 96%, 97%, 98%, 99%, or 100% sequence identity to SEQ ID NO: 32, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 70;
f) the first half antibody comprises a light chain sequence having at least about 90%, 91%, 92%, 93%, 94%, 95%, 96%, 97%, 98%, 99%, or 100% sequence identity to SEQ ID NO: 30 and a heavy chain sequence having at least about 90%, 91%, 92%, 93%, 94%, 95%, 96%, 97%, 98%, 99%, or 100% sequence identity to SEQ ID NO: 34, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 68;
g) the first half antibody comprises a light chain sequence having at least about 90%, 91%, 92%, 93%, 94%, 95%, 96%, 97%, 98%, 99%, or 100% sequence identity to SEQ ID NO: 44 and a heavy chain sequence having at least about 90%, 91%, 92%, 93%, 94%, 95%, 96%, 97%, 98%, 99%, or 100% sequence identity to SEQ ID NO: 46, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 70;
h) the first half antibody comprises a light chain sequence having at least about 90%, 91%, 92%, 93%, 94%, 95%, 96%, 97%, 98%, 99%, or 100% sequence identity to SEQ ID NO: 44 and a heavy chain sequence having at least about 90%, 91%, 92%, 93%, 94%, 95%, 96%, 97%, 98%, 99%, or 100% sequence identity to SEQ ID NO: 48, and the second half antibody comprises a light chain sequence having at least about 90%, 91%, 92%, 93%, 94%, 95%, 96%, 97%, 98%, 99%, or 100% sequence identity to SEQ ID NO: 60 and a heavy chain sequence having at least about 90%, 91%, 92%, 93%, 94%, 95%, 96%, 97%, 98%, 99%, or 100% sequence identity to SEQ ID NO: 68, optionally wherein a C-terminal lysine is missing from one or more chains.

In some embodiments, the antibody comprises first and second half antibodies, wherein
a) the first half antibody comprises a light chain sequence having at least about 90%, 91%, 92%, 93%, 94%, 95%, 96%, 97%, 98%, 99%, or 100% sequence identity to SEQ ID NO: 2 and a heavy chain sequence having at least about 90%, 91%, 92%, 93%, 94%, 95%, 96%, 97%, 98%, 99%, or 100% sequence identity to SEQ ID NO: 4, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 92;
b) the first half antibody comprises a light chain sequence having at least about 90%, 91%, 92%, 93%, 94%, 95%, 96%, 97%, 98%, 99%, or 100% sequence identity to SEQ ID NO: 2 and a heavy chain sequence having at least about 90%, 91%, 92%, 93%, 94%, 95%, 96%, 97%, 98%, 99%, or 100% sequence identity to SEQ ID NO: 6, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 90;
c) the first half antibody comprises a light chain sequence having at least about 90%, 91%, 92%, 93%, 94%, 95%, 96%, 97%, 98%, 99%, or 100% sequence identity to SEQ ID NO: 16 and a heavy chain sequence having at least about 90%, 91%, 92%, 93%, 94%, 95%, 96%, 97%, 98%, 99%, or 100% sequence identity to SEQ ID NO: 18, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 92;
d) the first half antibody comprises a light chain sequence having at least about 90%, 91%, 92%, 93%, 94%, 95%, 96%, 97%, 98%, 99%, or 100% sequence identity to SEQ ID NO: 16 and a heavy chain sequence having at least about 90%, 91%, 92%, 93%, 94%, 95%, 96%, 97%, 98%, 99%, or 100% sequence identity to SEQ ID NO: 20, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 90;
e) the first half antibody comprises a light chain sequence having at least about 90%, 91%, 92%, 93%, 94%, 95%, 96%, 97%, 98%, 99%, or 100% sequence identity to SEQ ID NO: 30 and a heavy chain sequence having at least about 90%, 91%, 92%, 93%, 94%, 95%, 96%, 97%, 98%, 99%, or 100% sequence identity to SEQ ID NO: 32, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 92;
f) the first half antibody comprises a light chain sequence having at least about 90%, 91%, 92%, 93%, 94%, 95%, 96%, 97%, 98%, 99%, or 100% sequence identity to SEQ ID NO: 30 and a heavy chain sequence having at least about 90%, 91%, 92%, 93%, 94%, 95%, 96%, 97%, 98%, 99%, or 100% sequence identity to SEQ ID NO: 34, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 90;
g) the first half antibody comprises a light chain sequence having at least about 90%, 91%, 92%, 93%, 94%, 95%, 96%, 97%, 98%, 99%, or 100% sequence identity to SEQ ID NO: 44 and a heavy chain sequence having at least about 90%, 91%, 92%, 93%, 94%, 95%, 96%, 97%, 98%, 99%, or 100% sequence identity to SEQ ID NO: 46, and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 92;
h) the first half antibody comprises a light chain sequence having at least about 90%, 91%, 92%, 93%, 94%, 95%, 96%, 97%, 98%, 99%, or 100% sequence identity to SEQ ID NO: 44 and a heavy chain sequence having at least about 90%, 91%, 92%, 93%, 94%, 95%, 96%, 97%, 98%, 99%, or 100% sequence identity to SEQ ID NO: 48,
and the second half antibody comprises a light chain sequence having at least about 90%, 91%, 92%, 93%, 94%, 95%, 96%, 97%, 98%, 99%, or 100% sequence identity to SEQ ID NO: 84 and a heavy chain sequence having at least about 90%, 91%, 92%, 93%, 94%, 95%, 96%, 97%, 98%, 99%, or 100% sequence identity to SEQ ID NO: 90,
optionally wherein a C-terminal lysine is missing from one or more chains.

In some embodiments, the antibody comprises first and second half antibodies, wherein the first half antibody comprises a light chain sequence of SEQ ID NO: 2 and a heavy chain sequence of SEQ ID NO: 4, and the second half antibody comprises a light chain sequence of SEQ ID NO: 60 and a heavy chain sequence of SEQ ID NO: 70, and optionally wherein a C-terminal lysine is missing from one or more heavy chains.

In some embodiments, the antibody comprises first and second half antibodies, wherein the first half antibody comprises a light chain sequence of SEQ ID NO: 2 and a heavy chain sequence of SEQ ID NO: 6, and the second half antibody comprises a light chain sequence of SEQ ID NO: 60 and a heavy chain sequence of SEQ ID NO: 68, and optionally wherein a C-terminal lysine is missing from one or more heavy chains.

In some embodiments, the antibody comprises first and second half antibodies, wherein the first half antibody comprises a light chain sequence of SEQ ID NO: 16 and a heavy chain sequence of SEQ ID NO: 18, and the second half antibody comprises a light chain sequence of SEQ ID NO: 60 and a heavy chain sequence of SEQ ID NO: 70, and optionally wherein a C-terminal lysine is missing from one or more heavy chains.

In some embodiments, the antibody comprises first and second half antibodies, wherein the first half antibody comprises a light chain sequence of SEQ ID NO: 16 and a heavy chain sequence of SEQ ID NO: 20, and the second half antibody comprises a light chain sequence of SEQ ID NO: 60 and a heavy chain sequence of SEQ ID NO: 68, and optionally wherein a C-terminal lysine is missing from one or more heavy chains.

In some embodiments, the antibody comprises first and second half antibodies, wherein the first half antibody comprises a light chain sequence of SEQ ID NO: 30 and a heavy chain sequence of SEQ ID NO: 32, and the second half antibody comprises a light chain sequence of SEQ ID NO: 60 and a heavy chain sequence of SEQ ID NO: 70, and optionally wherein a C-terminal lysine is missing from one or more heavy chains.

In some embodiments, the antibody comprises first and second half antibodies, wherein the first half antibody comprises a light chain sequence of SEQ ID NO: 30 and a heavy chain sequence of SEQ ID NO: 34, and the second half antibody comprises a light chain sequence of SEQ ID NO: 60 and a heavy chain sequence of SEQ ID NO: 68, and optionally wherein a C-terminal lysine is missing from one or more heavy chains.

In some embodiments, the antibody comprises first and second half antibodies, wherein the first half antibody comprises a light chain sequence of SEQ ID NO: 44 and a heavy chain sequence of SEQ ID NO: 46, and the second half antibody comprises a light chain sequence of SEQ ID NO: 60 and a heavy chain sequence of SEQ ID NO: 70, and optionally wherein a C-terminal lysine is missing from one or more heavy chains.

In some embodiments, the antibody comprises first and second half antibodies, wherein the first half antibody comprises a light chain sequence of SEQ ID NO: 44 and a heavy chain sequence of SEQ ID NO: 48, and the second half antibody comprises a light chain sequence of SEQ ID NO: 60 and a heavy chain sequence of SEQ ID NO: 68, and optionally wherein a C-terminal lysine is missing from one or more heavy chains.

In some embodiments, the antibody comprises first and second half antibodies, wherein the first half antibody comprises a light chain sequence of SEQ ID NO: 2 and a heavy chain sequence of SEQ ID NO: 4, and the second half antibody comprises a light chain sequence of SEQ ID NO: 84 and a heavy chain sequence of SEQ ID NO: 92, and optionally wherein a C-terminal lysine is missing from one or more heavy chains.

In some embodiments, the antibody comprises first and second half antibodies, wherein the first half antibody comprises a light chain sequence of SEQ ID NO: 2 and a heavy chain sequence of SEQ ID NO: 6, and the second half antibody comprises a light chain sequence of SEQ ID NO: 84 and a heavy chain sequence of SEQ ID NO: 90, and optionally wherein a C-terminal lysine is missing from one or more heavy chains.

In some embodiments, the antibody comprises first and second half antibodies, wherein the first half antibody comprises a light chain sequence of SEQ ID NO: 16 and a heavy chain sequence of SEQ ID NO: 18, and the second half antibody comprises a light chain sequence of SEQ ID NO: 84 and a heavy chain sequence of SEQ ID NO: 92, and optionally wherein a C-terminal lysine is missing from one or more heavy chains.

In some embodiments, the antibody comprises first and second half antibodies, wherein the first half antibody comprises a light chain sequence of SEQ ID NO: 16 and a heavy chain sequence of SEQ ID NO: 20, and the second half antibody comprises a light chain sequence of SEQ ID NO: 84 and a heavy chain sequence of SEQ ID NO: 90, and optionally wherein a C-terminal lysine is missing from one or more heavy chains.

In some embodiments, the antibody comprises first and second half antibodies, wherein the first half antibody comprises a light chain sequence of SEQ ID NO: 30 and a heavy chain sequence of SEQ ID NO: 32, and the second half antibody comprises a light chain sequence of SEQ ID NO: 84 and a heavy chain sequence of SEQ ID NO: 92, and optionally wherein a C-terminal lysine is missing from one or more heavy chains.

In some embodiments, the antibody comprises first and second half antibodies, wherein the first half antibody comprises a light chain sequence of SEQ ID NO: 30 and a heavy chain sequence of SEQ ID NO: 34, and the second half antibody comprises a light chain sequence of SEQ ID NO: 84 and a heavy chain sequence of SEQ ID NO: 90, and optionally wherein a C-terminal lysine is missing from one or more heavy chains.

In some embodiments, the antibody comprises first and second half antibodies, wherein the first half antibody comprises a light chain sequence of SEQ ID NO: 44 and a heavy chain sequence of SEQ ID NO: 46, and the second half antibody comprises a light chain sequence of SEQ ID NO: 84 and a heavy chain sequence of SEQ ID NO: 92, and optionally wherein a C-terminal lysine is missing from one or more heavy chains.

In some embodiments, the antibody comprises first and second half antibodies, wherein the first half antibody comprises a light chain sequence of SEQ ID NO: 44 and a heavy chain sequence of SEQ ID NO: 48, and the second half antibody comprises a light chain sequence of SEQ ID NO: 84 and a heavy chain sequence of SEQ ID NO: 90, and optionally wherein a C-terminal lysine is missing from one or more heavy chains.

In a further aspect, an antibody according to any of the above embodiments may incorporate any of the features, singly or in combination, as described in Sections 1-7 below.

### 1. Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or 0001 nM, and optionally is ≥10⁻¹³ M. (e.g. 10⁻⁸M or less, e.g. from 10⁻⁸M to 10⁻¹M, e.g., from 10⁻⁹ M to 10⁻¹³ M)

In some embodiments, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™. Packard) is added, and the plates are counted on a TOPCOUNT™ gamma counter (Packard) for ten minutes. Concentrations of each Fab that give less than or equal to 20% of maximal binding are chosen for use in competitive binding assays.

In some embodiments, Kd is measured using surface plasmon resonance assays using a BIACORE®-2000 or a BIACORE®-3000 (BIAcore, Inc., Piscataway, NJ) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (—0.2 μM) before injection at a flow rate of 5/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, F(ab′)2 fragments, dimeric single chain Fv, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

In some embodiments, the antibody is a diabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); US Pat. Nos. 5, 821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human 3-cell hybridoma technology are also described in Li et al., Proc. Natl. Acad Sci. USA 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). In some embodiments, the antibody comprises first and second half antibodies, wherein the first half antibody comprises a first heavy chain constant region comprising a knob mutation and the second heavy chain comprises a second heavy chain constant region comprising a hole mutation; or wherein the first half antibody comprises a first heavy chain constant region comprising a hole mutation and the second heavy chain comprises a second heavy chain constant region comprising a knob mutation. In some embodiments, the antibody is an IgG1 antibody and the knob mutation comprises a T366W mutation. In some embodiments, the antibody is an IgG1 antibody and the hole mutation comprises at least one, at least two, or three mutations selected from T366S, L368A, and Y407V. In some embodiments, the antibody is an IgG4 antibody and the knob mutation comprises a T366W mutation. In some embodiments, the antibody is an IgG4 antibody and the hole mutation comprises at least one, at least two, or three mutations selected from T366S, L368A, and Y407V mutations. The foregoing numbering of the positions of mutation(s) is EU numbering. The actual position(s) of the mutation(s) in a heavy chain sequence may vary, e.g., depending on the length of the preceding variable region, such as by up to 10 positions.

Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991). In some embodiments, the antibody is a diabody. Diabodies are antibody fragments with two antigen-binding sites that may be bivalent or bispecific. See, for example, EP 404,097; WO 1993/01161; Hudson et al., Nat. Med 9:129-134 (2003); and Hollinger et al., Proc. Natl. Acad Sci. USA 90: 6444-6448 (1993).

Triabodies and tetrabodies are described in Hudson et al., Nat. Med 9:129-134 (2003). In some embodiments, the antibody is a triabody. In some embodiments, the triabody comprises a first antigen recognition site, a second antigen recognition site, and a third antigen recognition site, wherein at least one of the antigen recognition sites differs from the other antigen recognition sites. In some embodiments, the triabody comprises first, second, and third antigen recognition sites that bind three different polyubiquitins. Each antigen recognition site can comprise a combination of HVRs or of a VL and VH discussed above.

In some embodiments, the antibody is a tetrabody. In some embodiments, the tetrabody comprises a first antigen recognition site, a second antigen recognition site, a third antigen recognition site, and a fourth antigen recognition site, wherein at least one or at least two of the antigen recognition sites differ from the other antigen recognition sites. In some embodiments, the tetrabody comprises first, second, and third antigen recognition sites that bind three different polyubiquitins. In some embodiments, the tetrabody comprises Each antigen recognition site can comprise a combination of HVRs or a combination of a VL and VH discussed above.

Engineered antibodies with three or more functional antigen binding sites, including “Octopus antibodies,” are also included herein (see, e.g. US 2006/0025576A1). The term octopus antibody is used in the sense of those discussed in US 2006/0025576A1 and is not meant to refer to an antibody produced by or obtained from an octopus.

The antibody or fragment herein also includes a “Dual Acting FAb” or “DAF” comprising two antigen binding sites that binds two different antigens (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc region variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, an antibody variant possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; K149 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In some embodiments, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In some such embodiments, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g., a Chinese Hamster Ovary (CHO) cell or lymphoid cell (e.g., Y0, NS0, Sp20 cell). In some embodiments, a method of making an antibody disclosed herein is provided, wherein the method comprises culturing a host cell comprising a nucleic acid encoding the antibody, as provided above, under conditions suitable for expression of the antibody, and optionally recovering the antibody from the host cell (or host cell culture medium).

In some embodiments, components of a multispecific antibody (e.g., a first half antibody and second half antibody) are expressed in separate cells or cell cultures and then combined in vitro. In other embodiments, all components of a multispecific antibody are expressed in the same cell or cell culture.

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻CHO cells (Urlaub et al., Proc. Natd. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### C. Assays

Antibodies provided herein may be identified, screened for, or characterized for their physical/chemical properties and/or biological activities by various assays known in the art.

In some aspects, an antibody is tested for its antigen binding activity, e.g., by known methods such as ELISA, FACS or Western blot.

In another aspect, competition assays may be used to identify an antibody that competes with any of the antibodies described herein. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized polyubiquitin is incubated with a solution comprising a first labeled antibody that binds thereto (e.g., any of the antibodies described herein) and a second unlabeled antibody that is being tested for its ability to compete with the first antibody for binding to the polyubiquitin. The second antibody may be present in a hybridoma supernatant. As a control, polyubiquitin is incubated in a solution comprising the first labeled antibody but not the second unlabeled antibody. After incubation under conditions permissive for binding of the first antibody to polyubiquitin, excess unbound antibody is removed, and the amount of label associated with immobilized polyubiquitin is measured. If the amount of label associated with immobilized polyubiquitin is substantially reduced in the test sample relative to the control sample, then that indicates that the second antibody is competing with the first antibody for binding to the polyubiquitin. See Harlow and Lane (1988) Antibodies: A Laboratory Manual ch.14 (Cold Spring Harbor Laboratory, Cold Spring Harbor, NY).

In some embodiments, a RIP1- or RIP2-K63-linked and/or linear ubiquitin chain bispecific antibody is used in a method for detection of selective ubiquitination of RIP1 or RIP2, respectively, polyubiquitinated with K63-linked and/or linear ubiquitin in a sample, e.g., a cellular or tissue sample, comprising contacting the sample with the antibody. In some embodiments, a RIP1- or RIP2-linear-linked ubiquitin chain bispecific antibody allows detection of selective ubiquitination of RIP1 or RIP2, respectively, polyubiquitinated with linear-linked ubiquitin, e.g., in the sample, e.g., a cellular or tissue sample.

In some embodiments, a method is provided for detecting ubiquitination of RIP1 or RIP2, such as K63-linked and/or linear polyubiquitin, using an antibody disclosed herein, by immunofluorescence, e.g., in a cellular or tissue sample. As demonstrated in the examples below, the detection can be in a time and/or signal dependent fashion. Usage of a single bispecific antibody can beneficially eliminate the necessity to tediously search for overlapping pattern of multiple antibodies and/or can increase the specificity and accuracy of cellular localization determination.

An antibody described herein can also be used in a method for assaying a tissue sample from a subject undergoing intestinal resection surgery to determine the level of RIP2 ubiquitination. As demonstrated in the examples, RIP2 can be heavily ubiquitinated with K63-linked and/or linear ubiquitin chains in samples from subjects having Crohn's Disease and ulcerative colitis, as opposed to lower ubiquitination levels in non-IBD control and diverticulitis samples.

Also provided herein is a method for identifying a subject as a candidate for a RIP2-targeting therapy, comprising contacting a sample from the subject with an antibody described herein and determining a level of a polyubiquitinated RIP2, such as K63-linked and/or linear polyubiquitinated RIP2. In some embodiments, the subject has or is suspected of having Crohn's Disease or ulcerative colitis. In some embodiments, the sample is a cellular or tissue sample.

In some embodiments, a method is provided for detecting ubiquitination of RIP1 or RIP2 comprising a branched, hybrid, or mixed polyubiquitin chain, e.g., wherein the branched, hybrid, or mixed polyubiquitin chain comprises one or more K63-linked and/or linear ubiquitin chains.

### D. Immunoconjugates

Immunoconjugates comprising an antibody disclosed herein conjugated to one or more cytotoxic agents are provided, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate).

Immunoconjugates allow for the targeted delivery of a drug moiety to a tumor or other diseased cell or tissue, and, in some embodiments intracellular accumulation therein, where systemic administration of unconjugated drugs may result in unacceptable levels of toxicity to normal cells (Polakis P. (2005) Current Opinion in Pharmacology 5:382-387).

Antibody-drug conjugates (ADC) are targeted chemotherapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107.

The ADC compounds include those with anticancer activity and/or with anti-inflammatory activity. In some embodiments, the ADC compounds include an antibody conjugated, i.e., covalently attached, to the drug moiety. In some embodiments, the antibody is covalently attached to the drug moiety through a linker. The antibody-drug conjugates (ADC) may selectively deliver an effective dose of a drug to tumor tissue whereby greater selectivity, i.e., a lower efficacious dose, may be achieved while increasing the therapeutic index (“therapeutic window”).

The drug moiety (D) of the antibody-drug conjugates (ADC) may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, pyrrolobenzodiazepine (PBD), nemorubicin and its derivatives, PNU-159682, anthracycline, duocarmycin, vinca alkaloid, taxane, trichothecene, CC1065, camptothecin, elinafide, and stereoisomers, isosteres, analogs, and derivatives thereof that have cytotoxic activity.

The drug moiety (D) of the ADC may include any compound, moiety or group that has anti-inflammatory effect. Exemplary drug moieties include, but are not limited to, nonsteroidal anti-inflammatory agents (NSAIDs), such as, ibuprofen, naproxen, diclofenac, diflunisal, etodolac, fenoprofen, flurbiprofen, indomethacin, ketorolac, mefenamic acid, meloxicam, nabumetone, oxaprozin, piroxicam, sulindac, and tolmetin; cox-2-inhibitors, such as celecoxib, rofecoxib, and valdecoxib; and stereoisomers, isosteres, analogs, and derivatives thereof that have anti-inflammatory activity.

### E. Methods and Compositions for Diagnostics and Detection

In some embodiments, a method described herein is useful for determining the presence of a polyubiquitinated protein in a sample. In some embodiments the sample is suspected of containing a polyubiqutinated protein. In some embodiments, the polyubiquitinated protein is a pro-inflammatory protein.

In some embodiments, a method described herein comprises exposing a sample to at least one multispecific antibody comprising a first half antibody comprising a first antigen binding site that binds to a polyubiquitin; and a second half antibody comprising a second antigen binding site that binds a pro-inflammatory protein. In some embodiments, the method comprises determining the binding of the at least one antibody to a polyubiquitinated protein in the sample.

In some embodiments, the polyubiqutinated protein comprises comprises a M1-linked polyubiquitin and/or a K63-linked polyubiquitin,

In some embodiments, a method described herein is useful for determining the presence of a polyubiquitinated protein in a sample suspected of containing a polyubiquitinated protein, wherein the polyubiquitinated protein is a pro-inflammatory protein and comprises a polyubiquitin, comprising exposing the sample to at least one multispecific antibody comprising a first half antibody comprising a first antigen binding site that binds to a polyubiquitin; and a second half antibody comprising a second antigen binding site that binds the pro-inflammatory protein, and determining the binding of the at least one antibody to a polyubiquitinated protein in the sample.

In some embodiments, a method described herein is useful for determining the presence of a polyubiquitinated protein in a sample suspected of containing a polyubiquitinated protein, wherein the polyubiquitinated protein is a pro-inflammatory protein and comprises a M1-linked polyubiquitin and/or a K63-linked polyubiquitin, comprising exposing the sample to at least one multispecific antibody comprising a first half antibody comprising a first antigen binding site that binds to a polyubiquitin; and a second half antibody comprising a second antigen binding site that binds the pro-inflammatory protein, and determining the binding of the at least one antibody to a polyubiquitinated protein in the sample.

In some embodiments, the pro-inflammatory protein is a component of one or more signaling complexes, which promote inflammation by mediating inflammatory cell death and/or release of pro-inflammatory cytokines, chemokines and danger-associated molecular patterns (DAMPs). In some embodiments, the pro-inflammatory protein is receptor-interacting protein kinase 1 (RIP1), receptor-interacting protein kinase 2 (RIP2), cellular inhibitors of apoptosis 1 and 2 (c-IAP1/2), Tumor Necrosis Factor Receptor 1 (TNFR1), linear ubiquitin chain assembly complex (LUBAC), and/or nuclear factor-kappa B (NF-κB) essential modulator (NEMO).

In some embodiments, the pro-inflammatory protein is RIP1 or RIP2.

In some embodiments, the pro-inflammatory protein has an elevated level of ubiquitination in the inflammatory state relative to the level of ubiquitination when not in the inflammatory state.

In some embodiments, the pro-inflammatory protein has a level of ubiquitination of at least 1-fold, 2-fold, 3-fold, 4-fold, 5-fold, 6-fold, 7-fold, 8-fold, 9-fold, 10-fold, 11-fold, 12-fold, 1-fold to 12-fold, 2-fold to 12-fold, 3-fold to 12-fold, 4-fold to 12-fold, 5-fold to 12-fold, 6-fold to 12-fold, 7-fold to 12-fold, 8-fold to 12-fold, 9-fold to 12-fold, 10-fold to 12-fold, or 11-fold to 12-fold in the inflammatory state relative to the level of ubiquitination when not in the inflammatory state.

In some embodiments, an elevated level of ubiquitination correlates to an increase in severity of an inflammatory disease state.

In some embodiments, an elevated level of ubiquitination in the inflammatory state relative to the level of ubiquitination when not in the inflammatory state.

In some embodiments, the pro-inflammatory protein is associated with an inflammatory disease, such as inflammatory bowel disease, Crohn's disease, diverticulitis, and ulcerative colitis.

In certain embodiments, the antibodies provided herein are useful for detecting the presence of RIP1 in a biological sample. In certain embodiments, the antibodies provided herein are useful for detecting the presence of RIP2 in a biological sample. The term “detecting” as used herein encompasses quantitative or qualitative detection. A “biological sample” comprises, e.g., a cell or tissue (e.g., biopsy material, including cancerous or potentially cancerous colon, colorectal, small intestine, endometrial, pancreatic, breast, lung, prostate, or ovarian tissue).

In some embodiments, an antibody disclosed herein is for use in a method of diagnosis or detection. In a further aspect, a method of detecting the presence of RIP1 in a biological sample is provided. In certain embodiments, the method comprises contacting the biological sample with an antibody as described herein under conditions permissive for binding of the antibody to RIP1 and detecting whether a complex is formed between the antibody and RIP1 in the biological sample. Such method may be an in vitro or in vivo method. In some embodiments, an antibody is used to select subjects eligible for therapy with an anti-RIP1 antibody, e.g., where RIP1 is a biomarker for selection of patients. In some embodiments, the biological sample is a cell or tissue (e.g., biopsy material, including cancerous or potentially cancerous tissue).

In some embodiments, an antibody disclosed herein is for use in a method of diagnosis or detection. In a further aspect, a method of detecting the presence of RIP2 in a biological sample is provided. In certain embodiments, the method comprises contacting the biological sample with an antibody as described herein under conditions permissive for binding of the antibody to RIP2 and detecting whether a complex is formed between the antibody and RIP2 in the biological sample. Such method may be an in vitro or in vivo method. In some embodiments, an antibody is used to select subjects eligible for therapy with an anti-RIP2 antibody, e.g., where RIP2 is a biomarker for selection of patients. In some embodiments, the biological sample is a cell or tissue (e.g., biopsy material, including cancerous or potentially cancerous tissue).

In some embodiments, a method of detecting a polyubiquitinated RIP1 or a polyubiquitinated RIP2 protein in a biological sample is provided. In certain embodiments, the method comprises contacting the biological sample with an antibody as described herein under conditions permissive for binding of the antibody to the polyubiquitinated RIP1 or the polyubiquitinated RIP2 protein, and detecting whether a complex is formed between the antibody and the RIP1 or RIP2 protein in the biological sample. Such method may be an in vitro or in vivo method. In some embodiments, an antibody is used to select subjects eligible for therapy with an antibody disclosed herein, e.g. where the polyubiquitinated RIP1 or the polyubiqutinated RIP2 protein is a biomarker for selection of patients. In some embodiments, the biological sample is a cell or tissue (e.g., biopsy material, including cancerous or potentially cancerous tissue).

In a further embodiment, an antibody disclosed herein is used in vivo to detect, e.g., by in vivo imaging, a polyubiquitinated RIP1 or a polyubiqutinated RIP2 protein, e.g., for the purposes of diagnosing, prognosing, or staging a disease, determining the appropriate course of therapy, or monitoring response to therapy. One method known in the art for in vivo detection is immuno-positron emission tomography (immuno-PET), as described, e.g., in van Dongen et al., The Oncologist 12:1379-1389 (2007) and Verel et al., J. Nucl. Med. 44:1271-1281 (2003). In such embodiments, a method is provided for detecting a polyubiquitinated RIP1 or polyubiquitinated RIP2 protein in a subject, the method comprising administering a labeled antibody to a subject, and detecting the labeled anti-RIP1 or anti-RIP2 antibody in the subject. In certain of such embodiments, the labeled antibody comprises (e.g., is conjugated to) a positron emitter, such as ⁶⁸Ga, ¹⁸F, ⁶⁴Cu, ⁸⁶Y, ⁷⁶Br, ⁸⁹Zr, and ¹²⁴I. In a particular embodiment, the positron emitter is ⁸⁹Zr. Nonlimiting exemplary methods of making and using ⁸⁹Zr-labeled antibodies are described, e.g., in PCT Publication No. WO 2011/056983. In some embodiments, the labeled antibody is a cysteine engineered antibody conjugated to one or more zirconium complexes. See, e.g., WO 2011/056983.

In further embodiments, a method of diagnosis or detection comprises contacting a first antibody disclosed herein which is immobilized to a substrate with a biological sample to be tested for the presence of a polyubiquitinated RIP1 or a polyubiquitinated RIP2 protein, exposing the substrate to a second antibody that binds or the polyubiquitinated RIP1 or polyubiquitinated RIP2 protein, and detecting whether the second antibody is bound to a complex between the first antibody and the polyubiquitinated RIP1 or the polyubiqutinated RIP2 protein in the biological sample (sometimes referred to as a sandwich assay). A substrate may be any supportive medium, e.g., glass, metal, ceramic, polymeric beads, slides, chips, and other substrates. In certain embodiments, a biological sample comprises a cell or tissue (e.g., biopsy material, including cancerous or potentially cancerous colon, colorectal, small intestine, endometrial, pancreatic or ovarian tissue). In certain embodiments, the first or second antibody is any of the antibodies described herein.

In certain embodiments, the antibodies disclosed herein are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment, a label is a positron emitter. Positron emitters include but are not limited to ⁶⁸Ga, ¹⁸F, ⁶⁴Cu, ⁸⁶Y, ⁷⁶Br, ⁸⁹Zr, and ¹²⁴I. In a particular embodiment, a positron emitter is ⁸⁹Zr.

Presence of a polyubiquitinated RIP1 or a polyubiqutinated RIP2 protein in a sample can be analyzed by a number of methodologies using an antibody disclosed herein, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), quantitative blood based assays (as for example Serum ELISA). Typical protocols for evaluating the status of proteins are found, for example in Ausubel et al., eds., 1995, Current Protocols In Molecular Biology, Unit 15 (Immunoblotting). Multiplexed immunoassays such as those available from Rules Based Medicine or Meso Scale Discovery (“MSD”) may also be used.

In some embodiments, a composition is provided that is substantially free of monospecific antibodies, unassembled half antibodies, or both monospecific antibodies and unassembled half antibodies. Monospecific antibodies are antibodies that do not comprise more than one type of antigen recognition site, e.g., antibodies with only one set of six CDRs or antibodies in which each set of six CDRs is identical. Antibodies in which a first set of CDRs varies only slightly from any other sets of CDRs, e.g., with respect to a small number of amino acid residues, wherein the differences do not result in preferential binding to a different antigen, are also considered monospecific. An unassembled half antibody is not stably associated (covalently or noncovalently) with another half antibody, e.g., appears as a single heavy/light chain unit when analyzed by an appropriate technique, such as size exclusion chromatography, mass spectrometry, or electrophoresis.

### F. Pharmaceutical Formulations

Pharmaceutical formulations of an antibody or immunoconjugate as described herein are prepared by mixing such antibody or immunoconjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In some aspects, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the antibodies or immunoconjugates provided herein may be used in methods, e.g., therapeutic methods.

In some aspects, an antibody or immunoconjugate disclosed herein for use as a medicament is provided. In further aspects, an antibody or immunoconjugate disclosed herein for use in a method of treatment is provided. In certain embodiments, an antibody or immunoconjugate disclosed herein for use in treating a cell-cycle-related disease or disorder is provided.

In some embodiments, an antibody or immunoconjugate disclosed herein is for use in treating a disease or disorder associated with aberrantly increased cell cycle progression and a disease or disorder associated with aberrantly decreased cell cycle progression is provided. In some embodiments, the disease or disorder associated with aberrantly increased cell cycle progression is cancer, such as colorectal cancer.

In some embodiments, an antibody or immunoconjugate disclosed herein is for use in treating an inflammatory disease, such as an inflammatory bowel disease is provided. In some embodiments, the inflammatory disease is selected from Crohn's disease, diverticulitis, and ulcerative colitis.

In some aspects, the use of an antibody or immunoconjugate disclosed herein in the manufacture or preparation of a medicament is provided.

An “individual” according to any of the above embodiments may be a human.

In some aspects, pharmaceutical formulations are provided, comprising any of the antibodies or immunoconjugate provided herein, e.g., for use in any of the above therapeutic methods. In some embodiments, a pharmaceutical formulation comprises any of the antibodies or immunoconjugates provided herein and a pharmaceutically acceptable carrier.

Antibodies or immunoconjugates provided herein can be used either alone or in combination with other agents in a therapy. For instance, an antibody or immunoconjugate provided herein may be co-administered with at least one additional therapeutic agent.

Such combination therapies noted above encompass combined administration (where two or more therapeutic agents are included in the same or separate formulations), and separate administration, in which case, administration of the antibody or immunoconjugate provided herein can occur prior to, simultaneously, and/or following, administration of the additional therapeutic agent and/or adjuvant.

An antibody or immunoconjugate (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or immunoconjugates would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or immunoconjugate need not be, but is optionally formulated with one or more agents currently used to prevent or treat the disorder in question. The effective amount of such other agents depends on the amount of antibody or immunoconjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or immunoconjugate (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the patient's clinical history and response to the antibody or immunoconjugate, and the discretion of the attending physician. The antibody or immunoconjugate is suitably administered to the patient at one time or over a series of treatments.

It is understood that any of the above formulations or therapeutic methods may be carried out using both an immunoconjugate and an antibody.

### H. Articles of Manufacture

In another aspect, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is an antibody or immunoconjugate disclosed herein. The label or package insert indicates that the composition is used for treating the condition of choice. Moreover, the article of manufacture may comprise (a) a first container with a composition contained therein, wherein the composition comprises an antibody or immunoconjugate; and (b) a second container with a composition contained therein, wherein the composition comprises a further cytotoxic or otherwise therapeutic agent. The article of manufacture in this embodiment may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

### III. Examples

The following are examples of methods and compositions provided herein. It is understood that various other embodiments may be practiced, given the general description provided above.

### A. Analytical Methods

Size Exclusion Chromatography—Multi-Angle Light Scattering. By way of example, 50 μg of antibody is injected onto a 3.5 μm, 7.8 mm×300 mm XBridge Protein BEH analytical SEC 200 Å column (Waters) at 1 mL/min using an Agilent 1260 Infinity HPLC with 20 mM histidine acetate, 300 mM NaCl, pH 5.5 as the mobile phase. Proteins eluted from the analytical SEC column are directly injected onto a Wyatt DAWN HELEOS II/Optilab T-rEX multi-angle light scattering detector to measure molar mass and polydispersity.

Mass Spectrometry. 30 μg of antibody is deglycosylated with 2 units of PNGaseF (NEB) at 37° C. overnight prior to mass spectrometry analysis. 2 μg of antibody is then injected onto a 3 μm, 4.6×50 mm reverse-phase chromatography PLRP-S column (Agilent) at 1 mL/min using an Agilent 1290 Infinity UHPLC. A 0-100% buffer B gradient over 3 minutes is performed with 0.05% trifluoroacetic acid (TFA) in water (buffer A) and 0.05% TFA in acetonitrile (buffer B), followed by a 100% buffer B wash for 1 minute. Proteins eluted from the reverse-phase column are directly injected onto an Agilent 6230 electrospray ionization time-of-flight mass spectrometer (ESI-TOF) for intact mass measurement.

### B. Antibody Cloning, Expression, and Annealing

Bispecific antibodies were generated using a knobs-into-holes heterodimerization approach. For a general discussion of this approach, see Merchant, A. M. et al. An efficient route to human bispecific IgG. Nat Biotechnol 16, 677-681, doi:10.1038/nbt0798-677 (1998).

Bispecific antibodies were designed using the following antibodies as the building blocks: an anti-M1 or linear polyubiquitin linkage-specific synthetic human antibody (clone 1F11/3F5/Y102L) (Matsumoto et al., J Mol Biol. 418(3-4), 134-44. doi: 10.1016/j.jmb.2011.12.053. Epub 2011 Dec. 29. PMID: 22227388 (2012))), an anti-K63 polyubiquitin linkage-specific synthetic human antibody (clone Apu3.A8) (Newton et al., Cell. 134(4), 668-78. doi: 10.1016/j.cell.2008.07.039. PMID: 18724939 (2008)), a murine anti-RIP1 antibody (BD Biosciences #610459), a murine anti-RIP2 antibody (Abcam #ab75257). As a control an anti-gD antibody recognizing an irrelevant protein was used.

T366W (knob) or T366S, L368A, and Y407V (hole) mutations were introduced into the CH3 domains. The knob and hole mutations were chosen to allow preferential heterodimerization of the respective heavy chains of the antibodies.

Briefly, sequences encoding the heavy chain variable domains were those of SEQ ID NO: 50 (for anti-M1 polyubiquitin linkage-specificity), SEQ ID NO: 36 (for anti-K63 polyubiquitin linkage-specificity), SEQ ID NO: 73 (for anti-RIP1), SEQ ID NO: 94 (for anti-RIP2) and a non-specific anti-gD control antibody. These variable domains were subcloned into a modified pRK vector (Genentech) containing the human IgG1 heavy chain constant domains with either the knob (T366W) or hole (T366S, L368A, and Y407V) mutations in the CH3 domain.

Due to the lengths of variable regions, the actual positions of the knob and hole mutations can vary slightly, e.g., by 1 to 10 positions. For example, in SEQ ID NOs: 4 and 6, the T to W and T to S substitutions, respectively, are reflected at the 369^{th} rather than the 366^{th} amino acid residue. It is understood that references to knob and hole mutations at positions such as 366, 368, and 407 of a heavy chain are to be interpreted with adjustments, if appropriate, in light of the length of the variable region.

The light chain variable domains were similarly subcloned into a modified pRK vector (Genentech) containing the human kappa light chain constant domain. The pRK vector carries a constitutive strong signal peptide for extracellular expression in mammalian cells. The anti-M1 antibodies were cloned as both knob and hole mutants (encoding heavy chains of SEQ ID NOs: 46 and 48, respectively) as were the anti-K63 antibodies (encoding heavy chains of SEQ ID NOs: 32 and 34, respectively). The anti-RIP1 and anti-RIP2 antibodies were cloned as hole mutants (encoding SEQ ID NOs: 70 and 92, respectively) and the anti-gD antibody was cloned as a knob mutant.

The sequence table also provides knob and hole mutants of an anti-K11 heavy chain (SEQ ID NOs: 4 and 6, respectively); knob and hole mutants of an anti-K48 heavy chain (SEQ ID NOs: 18 and 20, respectively); as well as knob mutant of the anti-RIP1 heavy chain (SEQ ID NO: 68) and knob mutant of the anti-RIP2 heavy chain (SEQ ID NO: 90).

To preserve the pairing of the cognate light and heavy chains we expressed either the knob or hole heavy chain mutants with their respective light chains separately in CHO cells and affinity purified them individually (data not shown). Light chain and heavy chain plasmids for a given knob or hole half antibody were transiently co-transfected into CHO cells using PEI as previously described (see Wong, A. W., Baginski, T. K. & Reilly, D. E. Enhancement of DNA uptake in FUT8-deleted CHO cells for transient production of afucosylated antibodies. Biotechnol Bioeng 106, 751-763, doi:10.1002/bit.22749 (2010)). Half antibodies were purified over MabSelect SuRe resin (GE Healthcare), eluted with 50 mM sodium citrate, 150 mM NaCl, pH 3.0, followed by pH adjustment to 5.0 with 10% (v/v) of 200 mM arginine, 137 mM succinate, pH 9.0.

The affinity-purified knob and hole half antibodies were characterized (not shown), consistent with previously described knob and hole antibodies. See, e.g., Shatz, W. et al. MAbs 5, 872-881, doi:10.4161/mabs.26307 (2013). Their identities were confirmed by mass spectrometry.

Bispecific antibodies were assembled from half antibodies in vitro using annealing, reduction, and oxidation. The anti-RIP1/anti-M1 bispecific antibody was assembled in vitro from the affinity purified anti-M1 knob and anti-RIP1 hole antibodies using a modified version of the previously described method of annealing, reduction, and oxidation (Shatz, W. et al. MAbs 5, 872-881, doi:10.4161/mabs.26307 (2013)). Similarly, the anti-RIP1/anti-K63 bispecific antibody was assembled from anti-K63 knob and anti-RIP1 hole antibodies; the anti-RIP2/anti-M1 bispecific antibody was assembled from anti-M1 knob and anti-RIP2 hole antibodies; and the anti-RIP2/anti-K63 bispecific antibody was assembled from anti-K63 knob and anti-RIP2 hole antibodies. Briefly, the desired knob and hole half antibodies were mixed at a 1:1 mass ratio and the pH of the mixture was adjusted to ˜8 with 15% (v/v) of 800 mM arginine, pH 10.0. A 200-fold molar excess of reduced glutathione (Sigma Aldrich) in 800 mM arginine, pH 10.0 was added and the assembly reaction was incubated at room temperature for 72 hours with exposure to air to allow annealing of the knob and hole half antibodies and formation of the hinge disulfides. Anti-M1/anti-K63, anti-RIP1/gD, anti-RIP2/gD, anti-M1/gD, and anti-K63/gD control bispecific antibodies were similarly assembled.

### C. Antibody Purification and Characterization

In vitro-assembled bispecific antibodies were purified by hydrophobic interaction chromatography (HIC) using a modified version of the previously described method. See Yau et al., Cell. 171(4):918-933.e20. doi: 10.1016/j.cell.2017.09.040. Epub 2017 Oct. 12. PMID: 29033132; PMCID: PMC5669814 (2017). Briefly, 3.8 M ammonium sulfate was added to the assembly reaction to a final concentration of at least 1 M, the reaction was filtered with a 0.22 μm filter, loaded onto a 5 μm, 7.8×75 mm ProPac HIC-10 column (Dionex). The column was then washed with buffer A (20 mM sodium acetate, pH 5.0), and a 0-100% buffer B (25 mM sodium phosphate, pH 6.5, 25% isopropanol) linear gradient over 40 column volumes (CVs) was performed to separate the bispecific antibody from any unreacted half antibodies or aggregated protein. Identity of the eluting peaks was monitored by SDS-PAGE and mass spectrometry (see below for method details), and fractions corresponding to the bispecific peak were pooled.

The bispecific antibodies were further purified by cation-exchange chromatography (CEX). Briefly, the HIC pooled material was either dialyzed into 20 mM sodium acetate, pH 5.0 or the pH was lowered to ˜5 with the addition of ⅙^{th} volume of 1 M sodium acetate, pH 5.0 and then diluted with water to a final concentration of 13 mM sodium acetate. The antibodies were then loaded onto a 10 m Mono S 5/50 GL column (GE Healthcare), and washed with buffer A (20 mM sodium acetate, pH 5.0). A 0-100% buffer B (20 mM sodium acetate, pH 5.0, 1 M NaCl) linear gradient over 40 CVs was performed and the desired fractions pooled. The purified bispecific antibodies were formulated in either 20 mM histidine acetate, 240 mM sucrose, 0.02% TWEEN®-20, pH 5.5 or 20 mM histidine acetate, 150 mM NaCl, pH 5.5.

LC-MS was used to confirm the identity of the purified, annealed species. To reduce heterogeneity the antibodies were deglycosylated with PNGaseF before analysis.

### D. RIP1-K63 Ubiquitin Chain Bispecific Antibodies Recognize Protein Modifications

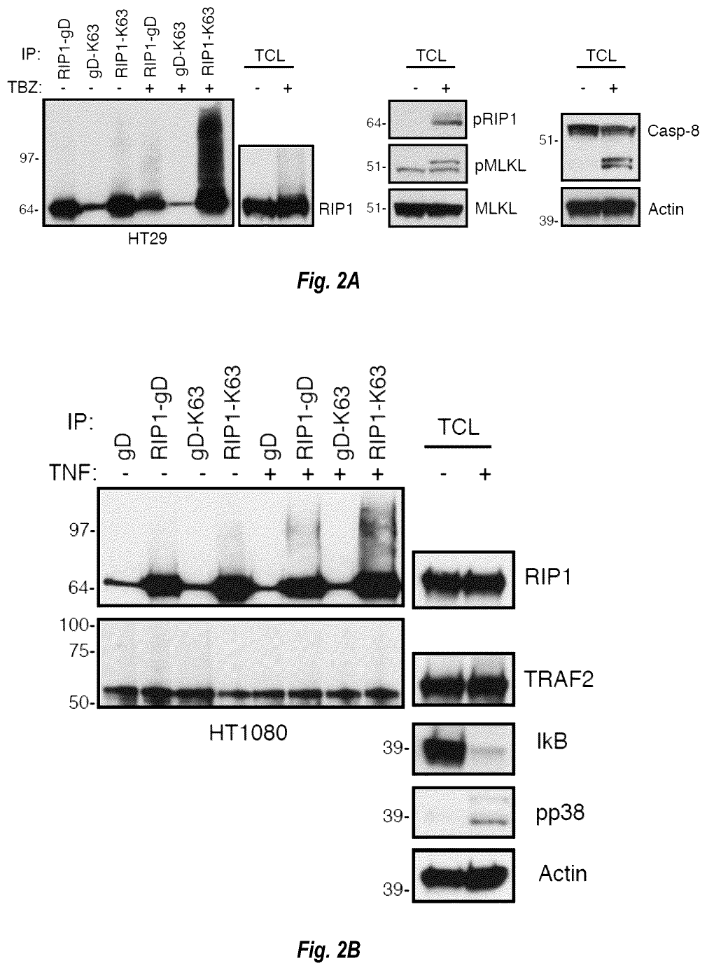

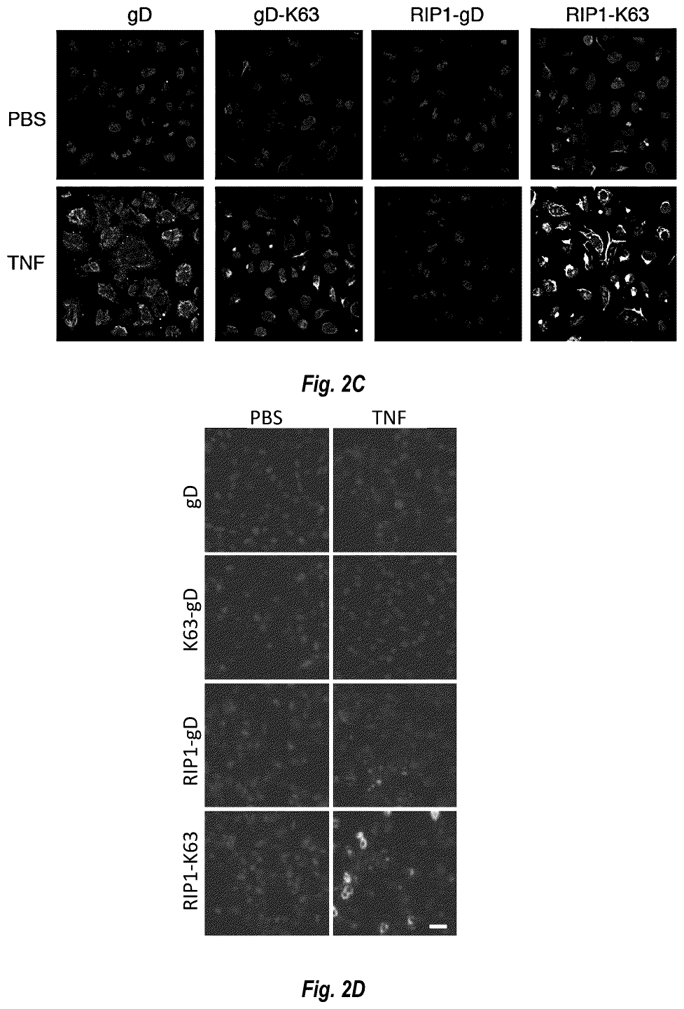

Human colon carcinoma HT29 cells were treated with the combination of TNF (20 ng/ml), the IAP antagonist BV6 (2 μM) and the pancaspase inhibitor zVAD (20 μM) (collectively, TBZ) for 2 hours, as also described generally in Almagro et al., Cell Death and Differentiation, 24:26-37 (2017). Cells were lysed in 6M urea buffer and immunoprecipitated using indicated RIP1-ubiquitin chain or control bispecific antibodies. Total cell lysates (TCL) and immunoprecipitated proteins (IP) were probed with the indicated antibodies, as shown in FIG. 2A.

Fibrosarcoma HT1080 cells were treated with PBS or TNF (100 ng/ml) for 7 min. Cells were lysed in 6M urea buffer and immunoprecipitated using indicated RIP1-ubiquitin chain or control bispecific antibodies. Total cell lysates and immunoprecipitated proteins were probed with the indicated antibodies, as shown in FIG. 2B.

A549 cells were treated with PBS or TNF (500 ng/ml) for 7 min. Cells were fixed in 4% paraformaldehyde for 30 min at room temperature (RT), incubated in 6M urea buffer without Triton (30 min, RT), permeabilized with 0.25% Triton (10 min, RT) and then stained using indicated RIP1-ubiquitin chain bispecific or control antibodies, followed by anti-human IgG-Alexa 488 secondary antibody. Hoechst 33258 was used for nuclear staining. Mounted slides were analyzed with Leica SP8 confocal microscopy. All images were collected uniformly using the same settings. These results are shown in FIG. 2C.

HT1080 cells were treated with PBS or TNF (500 ng/ml) for 7 min. Cells were fixed, incubated in 6 M urea buffer (30 min, RT), permeabilized and then stained using indicated RIP1-ubiquitin chain bispecific or control antibodies, followed by anti-human IgG-Alexa 488 secondary antibody. Hoechst 33258 was used for nuclear staining. All images were collected uniformly using same settings [scale bar 25 μm]. These results are shown in FIG. 2D.

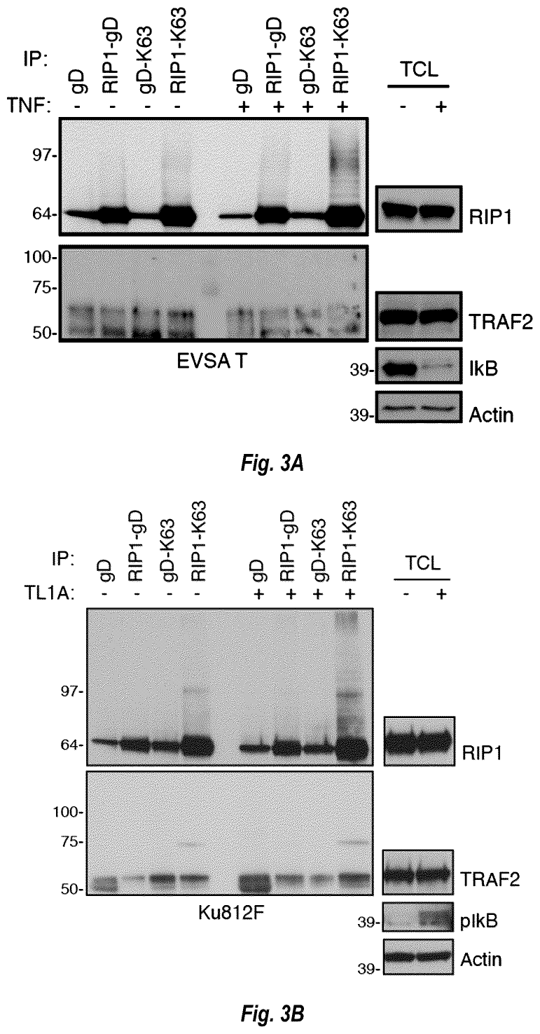

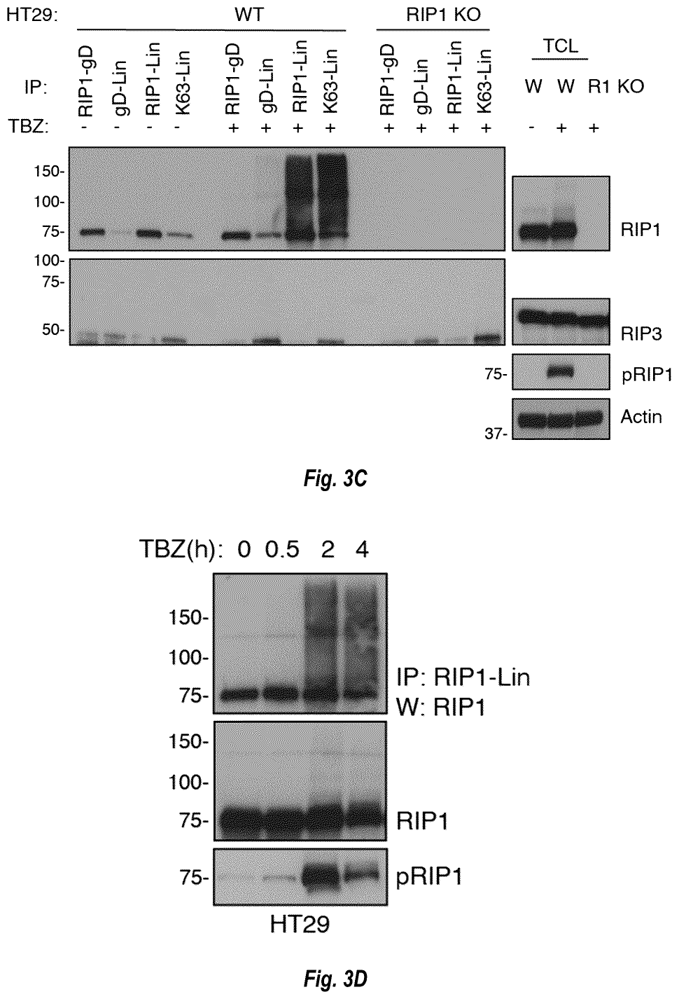

EVSA T cells were treated with PBS or TNF (100 ng/ml) for 7 min. Cells were lysed in 6M urea buffer and immunoprecipitated using indicated RIP1-ubiquitin chain or control bispecific antibodies. Total cell lysates and immunoprecipitated proteins were probed with the indicated antibodies. These results are shown in FIG. 3A.

Ku812F cells were treated with PBS or Flag-TL1A (10 ug) crosslinked with anti-Flag AB for 10 min. Cells were lysed in 6M urea buffer and immunoprecipitated using indicated RIP1-ubiquitin chain or control bispecific antibodies. Total cell lysates and immunoprecipitated proteins were probed with the indicated antibodies. These results are shown in FIG. 3B.

This shows that RIP1-K63 ubiquitin chain bispecific antibodies recognize K63 chain-ubiquitinated RIP1 (FIG. 2A-2D) and RIP1 modified with K63-linked ubiquitin chains (FIG. 3A-3B).

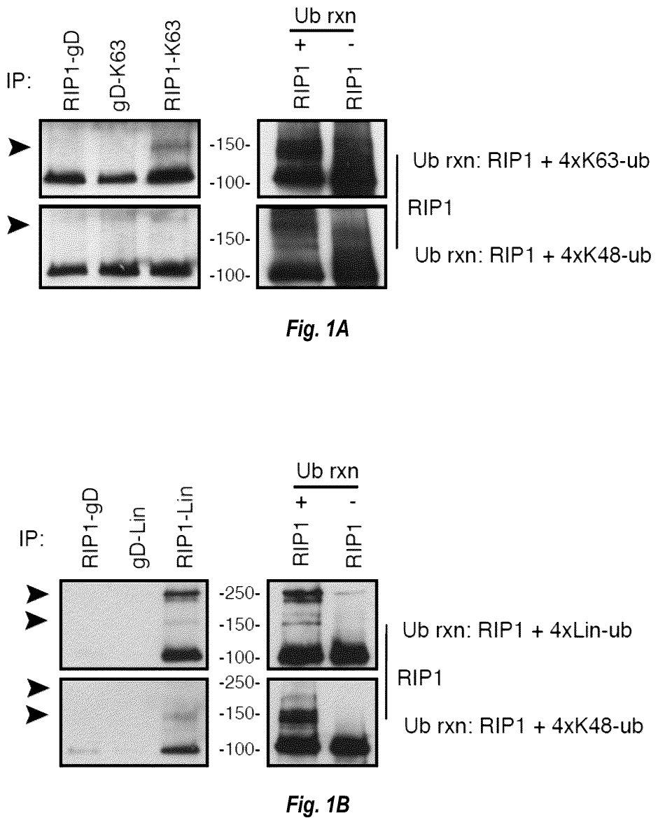

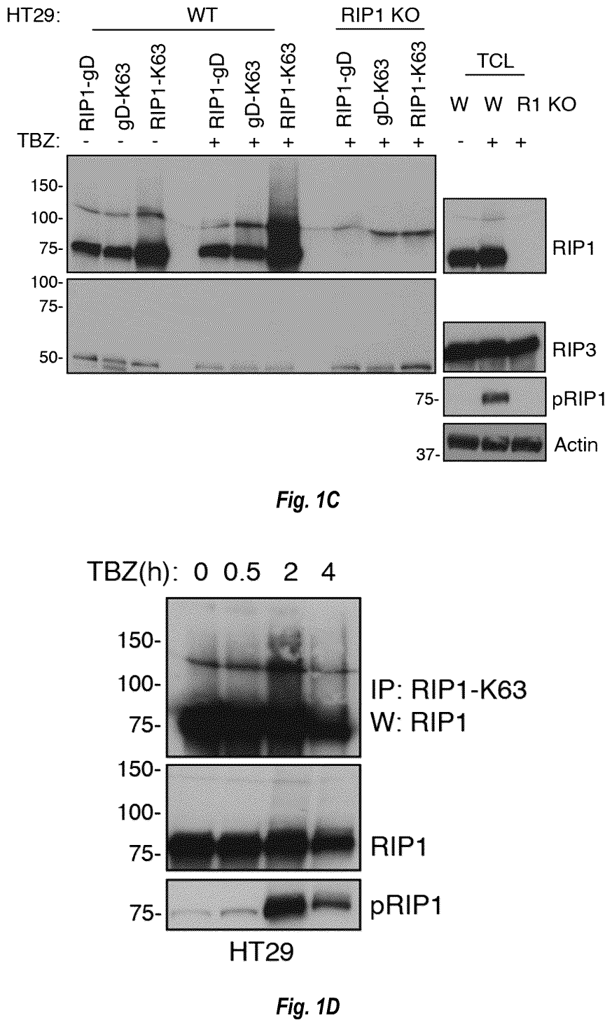

This is also shown in FIGS. 1A-D. The specificity of RIP1-K63 bispecific antibodies was verified with in vitro ubiquitinated RIP1, where either K63- or K48-tetraubiquitin molecules were used to modify RIP1. Following ubiquitination, RIP1 was incubated with the indicated bispecific antibodies in the presence of 6 M urea to disrupt any non-covalent associations, precipitated and examined by western blotting. The RIP1-K63 bispecific antibody immunoprecipitated K63-ubiquitin chain modified RIP1 but not K48-ubiquitin chain modified RIP1.

### E. RIP1-Lin Ubiquitin Chain Bispecific Antibodies Recognize Protein Modifications

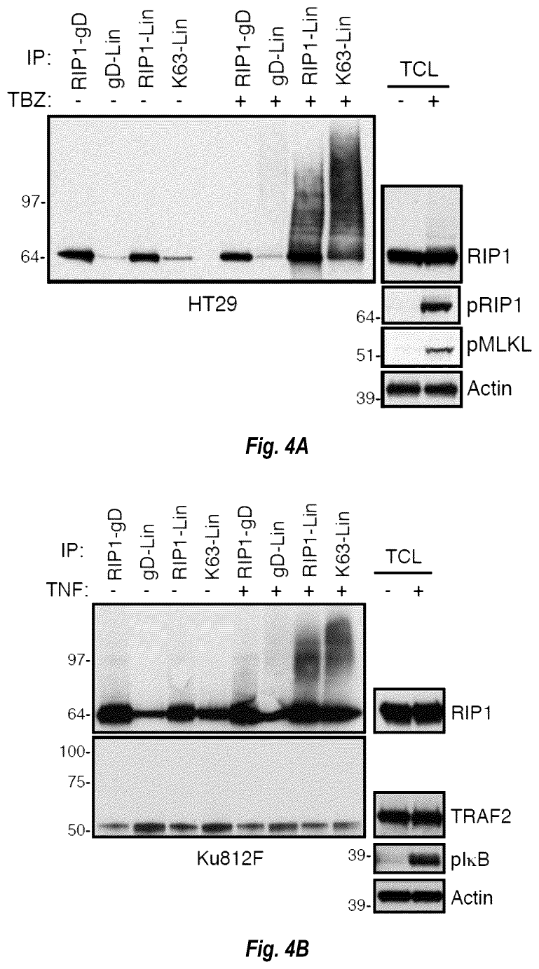

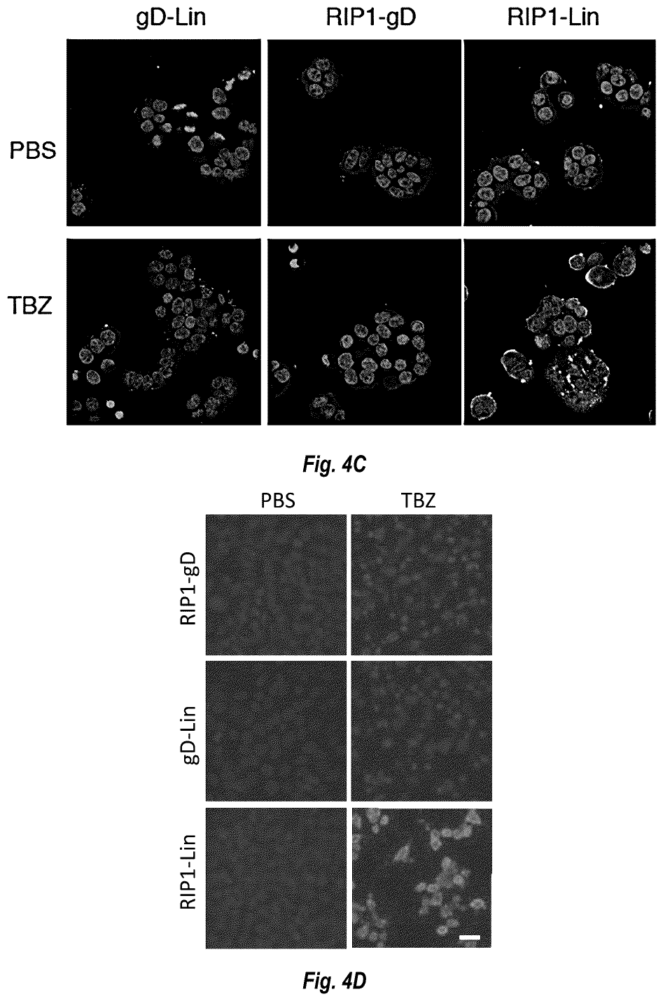

HT29 cells were treated with PBS or TNF (20 ng/ml), BV6 (2 μM) and zVAD (20 μM) for 2 hours, as described in Example D. Cells were lysed in 6M urea buffer and immunoprecipitated using indicated RIP1-ubiquitin, K63-linear ubiquitin chain or control bispecific antibodies. Total cell lysates and immunoprecipitated proteins were probed with the indicated antibodies, as shown in FIG. 4A.

Ku812F cells were treated with PBS or TNF (100 ng/ml) for 10 min. Cells were lysed in 6M urea buffer and immunoprecipitated using indicated RIP1-ubiquitin chain, K63-linear ubiquitin chain or control bispecific antibodies. Total cell lysates and immunoprecipitated proteins were probed with the indicated antibodies, as shown in FIG. 4B.

HT29 cells were treated with PBS or TNF (100 ng/ml), BV6 (2 μM) and zVAD (20 μM) for 2.5 hours. Cells were fixed in 4% paraformaldehyde for 30 min at room temperature (RT), incubated in 6M urea buffer without Triton (30 min, RT), permeabilized with 0.25% Triton (10 min, RT) and then stained using indicated RIP1-ubiquitin chain bispecific or control antibodies, followed by anti-human IgG-Alexa 488 secondary antibody. Hoechst 33258 was used for nuclear staining. Mounted slides were analyzed with Leica SPE confocal microscopy. All images were collected uniformly using same settings. These results are shown in FIG. 4C.

Mouse embryonic fibroblasts (MEF) cells were treated with PBS or TBZ (TNF (100 ng/ml), BV6 (1 μM) and zVAD (20 μM)) for 2.5 h. Cells were fixed, incubated in 6 M urea buffer (30 min, RT), permeabilized and then stained using the indicated RIP1-ubiquitin chain bispecific or control antibodies, followed by anti-human goat F(ab′)2 fragment antibody conjugated to Cy3. Hoechst 33258 was used for nuclear staining. All images were collected uniformly using the same settings [scale bar 25 μm]. These results are shown in FIG. 4D.

These results show that RIP1-Lin ubiquitin chain bispecific antibodies recognize linear chain-ubiquitinated RIP1. On the contrary, RIP1-gD and gD-Lin antibodies, or K63-Lin antibody without stimulation, did not immunoprecipitate ubiquitinated RIP1.

This is also shown in FIG. 1B, which shows that in vitro ubiquitination of RIP1 with tetra-K48 or linear-tetra ubiquitin molecules followed by immunoprecipitation in 6 M urea buffer demonstrated the ability of RIP1-Lin bispecific antibody to selectively immunoprecipitate RIP1 modified with linear ubiquitin chains, but not with K48-linked ubiquitin chains.

### F. RIP1-Lin and K63-Lin Ubiquitin Chain Bispecific Antibodies Recognize Modified RIP1

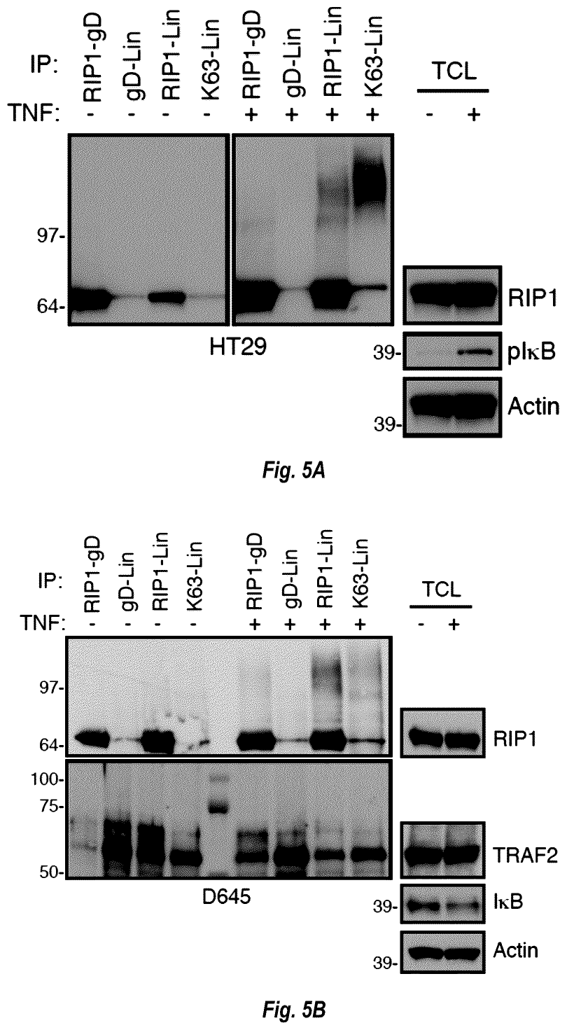

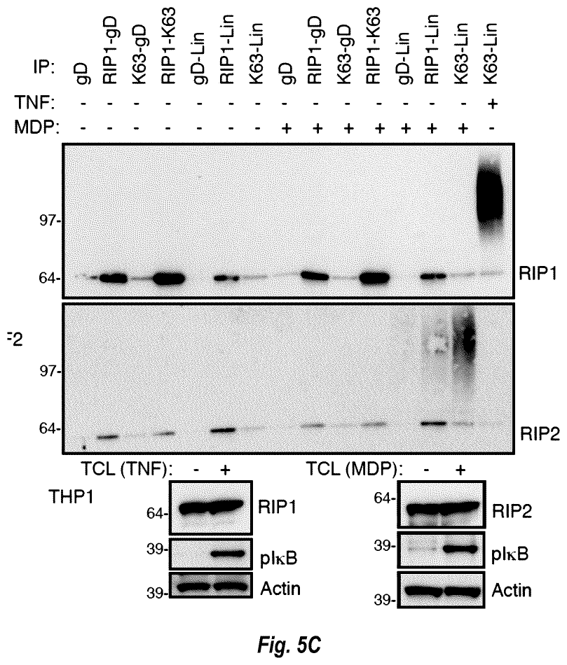

HT29 cells were treated with PBS or TNF (50 ng/ml) for 7 min. Cells were lysed in 6M urea buffer and immunoprecipitated using the indicated RIP1-ubiquitin chain, K63-linear ubiquitin chain or control bispecific antibodies. Total cell lysates and immunoprecipitated proteins were probed with the indicated antibodies, as shown in FIG. 5A.

D645 cells were treated with PBS or TNF (100 ng/ml) for 7 min. Cells were lysed in 6M urea buffer and immunoprecipitated using indicated RIP1-ubiquitin chain, K63-linear ubiquitin chain or control bispecific antibodies. Total cell lysates and immunoprecipitated proteins were probed with indicated antibodies, as shown in FIG. 5B.

THP1 cells were treated with PBS, TNF (100 ng/ml, 5 min) or MDP (1 μg/ml, 30 min). Cells were lysed in 6M urea buffer and immunoprecipitated using indicated RIP1-ubiquitin, K63-linear ubiquitin chain bispecific or control antibodies. Total cell lysates and immunoprecipitated proteins were probed with indicated antibodies, as shown in FIG. 5C.

Thus, RIP1-K63 and RIP1-Lin bispecific antibodies selectively recognize RIP1 modified with K63-linked and K63-Lin ubiquitin linked chains, respectively, but not RIP2 or TRAF2.

K63-Lin bispecific antibody also recognizes modified RIP1, as well as modified RIP2, but there is no RIP1-specific component or RIP-2 specific component in this antibody.

### G. RIP1-Ubiquitin Chain Bispecific Antibodies Recognize RIP1 Ubiquitinated In Vivo

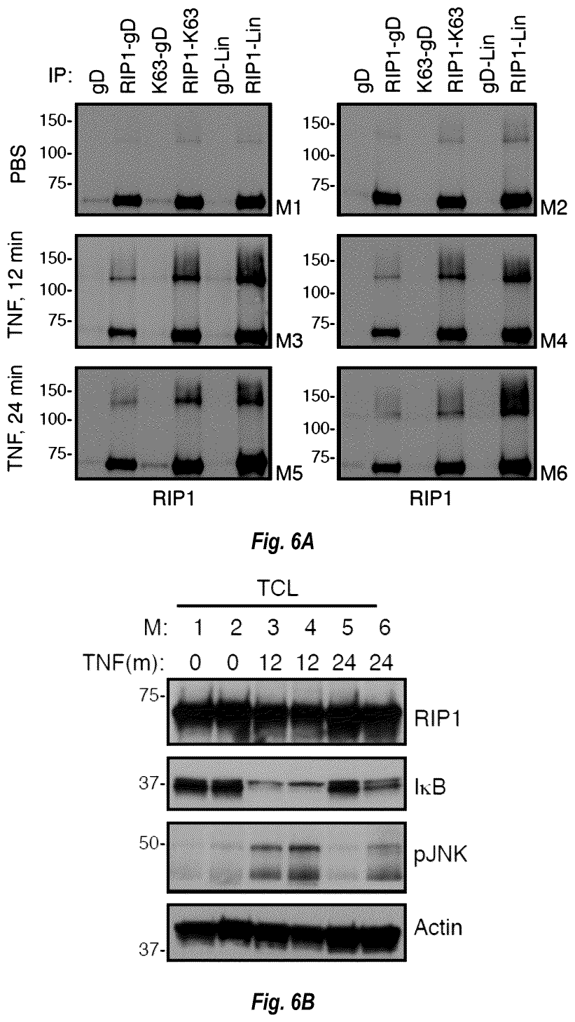

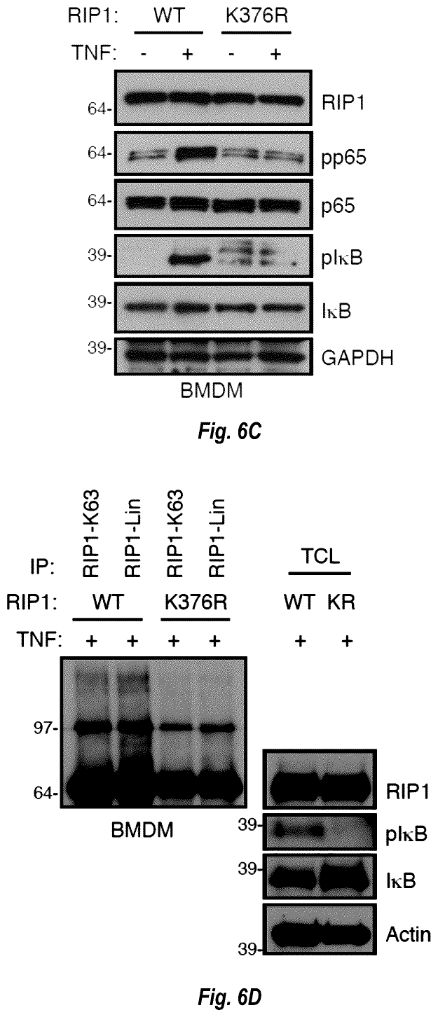

Mice were treated with PBS (mice M1 and M2) or TNF (500 μg/ml) for 12 min (mice M3 and M4) or 24 min (mice M5 and M6). Small intestines from indicated mice were lysed in 6M urea buffer and immunoprecipitated using indicated RIP1-ubiquitin chain or control bispecific antibodies. Immunoprecipitated proteins (FIG. 6A) and total cell lysates proteins (FIG. 6B) were probed with the indicated antibodies. This demonstrates that RIP1-ubiquitin chain bispecific antibodies recognize RIP1 ubiquitinated in vivo with K63-linked and linear ubiquitin chains.

Bone marrow derived macrophages (BMDMs) were isolated from mice with wild-type RIP1 or RIP1 K376R knock-in mice, treated with TNF for 5 minutes, lysed in 6M urea buffer and immunoprecipitated using indicated RIP1-ubiquitin chain or control bispecific antibodies. Detection of total cell lysates proteins (FIG. 6C) and immunoprecipitated proteins (FIG. 6D) was done using the indicated antibodies. This demonstrates that impaired K63-linked and linear ubiquitination of RIP1 in RIP1 K376R knock-in mice derived BMDMs can be detected using RIP1-ubiquitin chain bispecific antibodies.

### H. RIP2-Ubiquitin Chain Bispecific Antibodies Recognize Modified RIP2

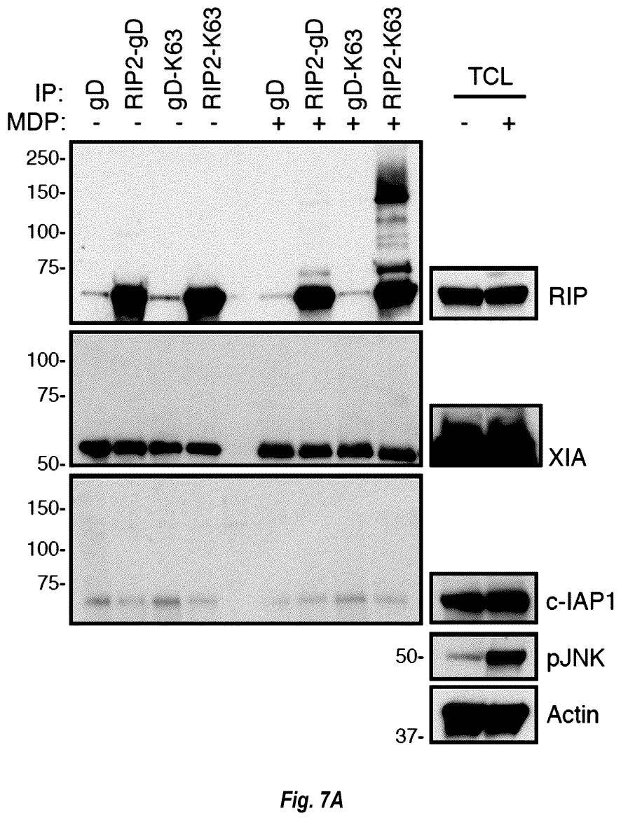

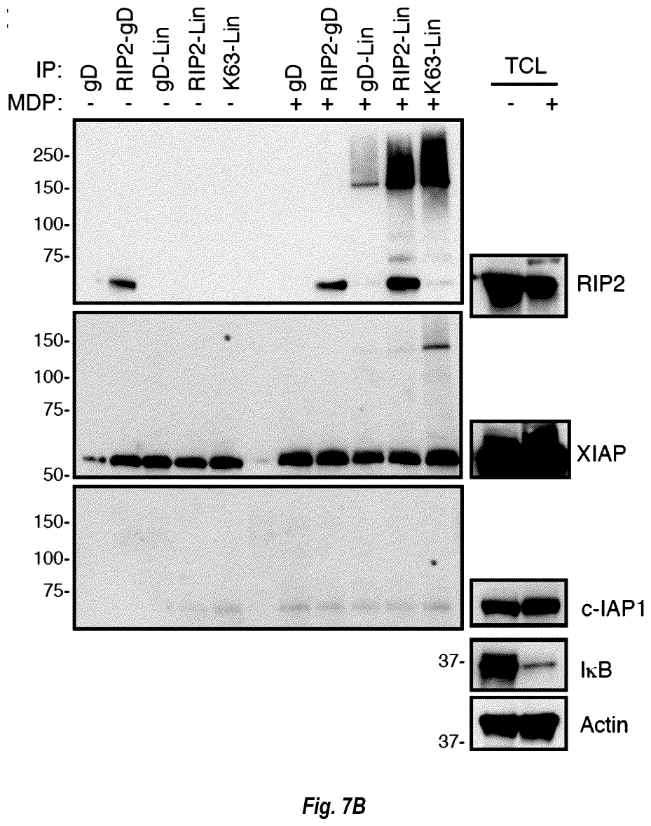

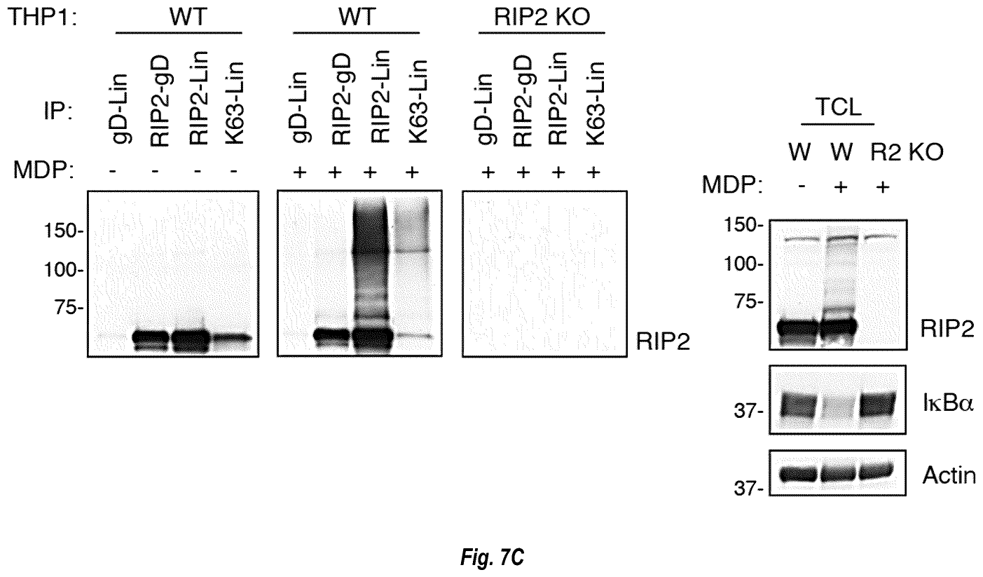

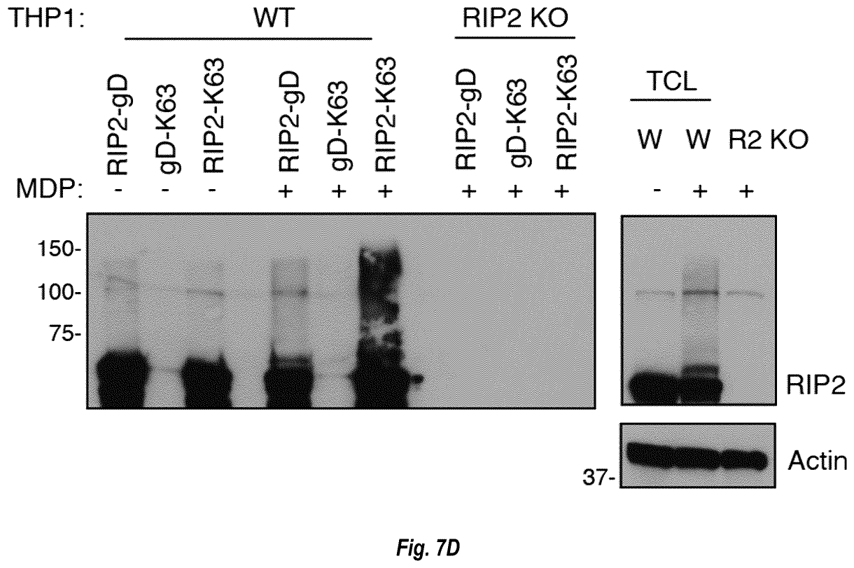

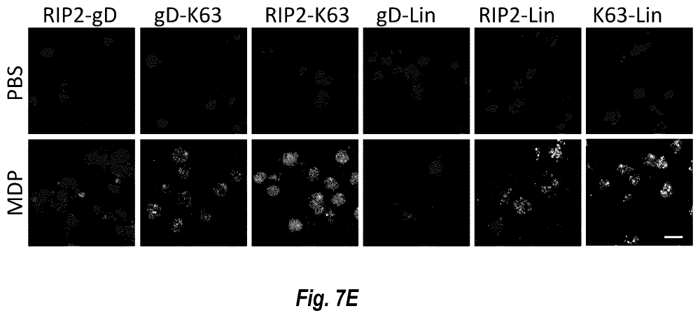

THP1 cells were treated with PBS or the NOD2 signaling activator MDP (1 μg/ml, 30 min). Cells were lysed in 6M urea buffer and immunoprecipitated using indicated RIP2-ubiquitin chain or control bispecific antibodies. Total cell lysates and immunoprecipitated proteins were probed with indicated antibodies, as shown in FIG. 7A. This resulted in detection of RIP2 K63-linked ubiquitination in a stimulus dependent fashion only when both the RIP2 and K63 arms were present in the bispecific (RIP2-K63), but not with either control bispecific containing only one of the relevant arms (RIP2-gD or K63-gD).

THP1 cells were treated with PBS or MDP (1 μg/ml, 30 min). Cells were lysed in 6M urea buffer and immunoprecipitated using indicated RIP2-ubiquitin, K63-linear ubiquitin chain bispecific or control antibodies. Total cell lysates and immunoprecipitated proteins were probed with indicated antibodies, as shown in FIG. 7B. RIP2-Lin, but not RIP2-gD or gD-Lin antibodies can successfully immunoprecipitate RIP2 modified by linear ubiquitin, but not XIAP or c-IAP1. K63-Lin bispecific antibody efficiently captures RIP2, and to some degree XIAP, modified by mixed and/or branched K63-linked and linear ubiquitin chains.

THP1 cells were treated with PBS or MDP (1 μg/ml, 30 min). Cells were fixed in 4% paraformaldehyde for 30 min at room temperature (RT), incubated in 6M urea buffer without Triton (30 min, RT), permeabilized with 0.25% Triton (10 min, RT) and then stained using indicated RIP2-ubiquitin chain bispecific or control antibodies, followed by anti-human IgG-Alexa 488 secondary antibody. Hoechst 33258 was used for nuclear staining. Western blot analyses of TCL and immunoprecipitated proteins obtained with the indicated antibodies using WT (wild-type, W) and RIP2 KO (knockout) THP1 cells are shown in FIG. 7C. This result confirms that the RIP2-ubiquitin chain bispecific antibodies recognize RIP2 ubiquitinated by linear chains and K63-Lin ubiquitin linked chains. Western blot analyses of TCL and immunoprecipitated proteins obtained with the indicated antibodies using WT (wild-type, W) and RIP2 KO (knockout) THP1 cells are shown in FIG. 7D. Mounted slides were analyzed with Leica SPE confocal microscopy. All images were collected uniformly using same settings. These results are shown in FIG. 7E. The RIP2-K63, RIP2-Lin and K63-Lin, but not RIP2-gD, gD-K63 or gD-Lin bispecific antibodies revealed stimulus dependent localization of K63-linked and linear RIP2 ubiquitination in cells by immunofluorescence. The immunofluorescence detected with the K63-Lin antibody is likely predominantly but not exclusively the results of RIP2 ubiquitination since this antibody weakly immunoprecipitated XIAP in MDP treated cells.

This demonstrates that RIP2-ubiquitin chain bispecific antibodies recognize K63 and linear chain-ubiquitinated RIP2. In contrast, single-arm (RIP2-gD, K63-gD, Lin-gD) antibodies do not recognize ubiquitinated RIP2, and even RIP2-K63, RIP2-Lin and K63-Lin bispecific antibodies recognize K63- and linear chain-ubiquitinated RIP2 only after treatment with pathway-relevant stimulus (MDP).

I. RIP2 Ubiquitination in Crohn's disease and Ulcerative Colitis Patient Samples

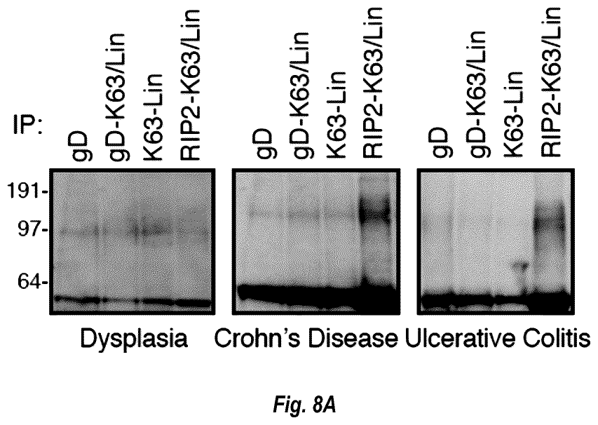

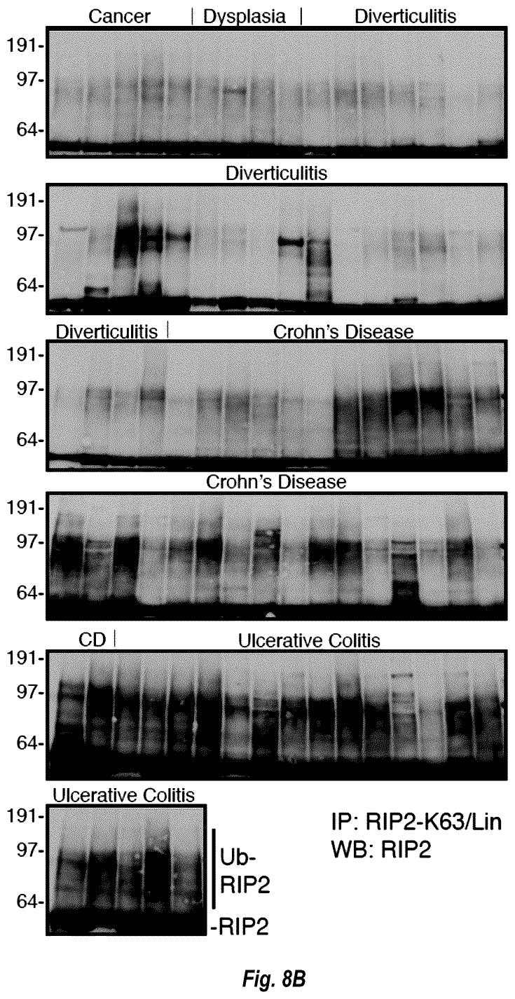

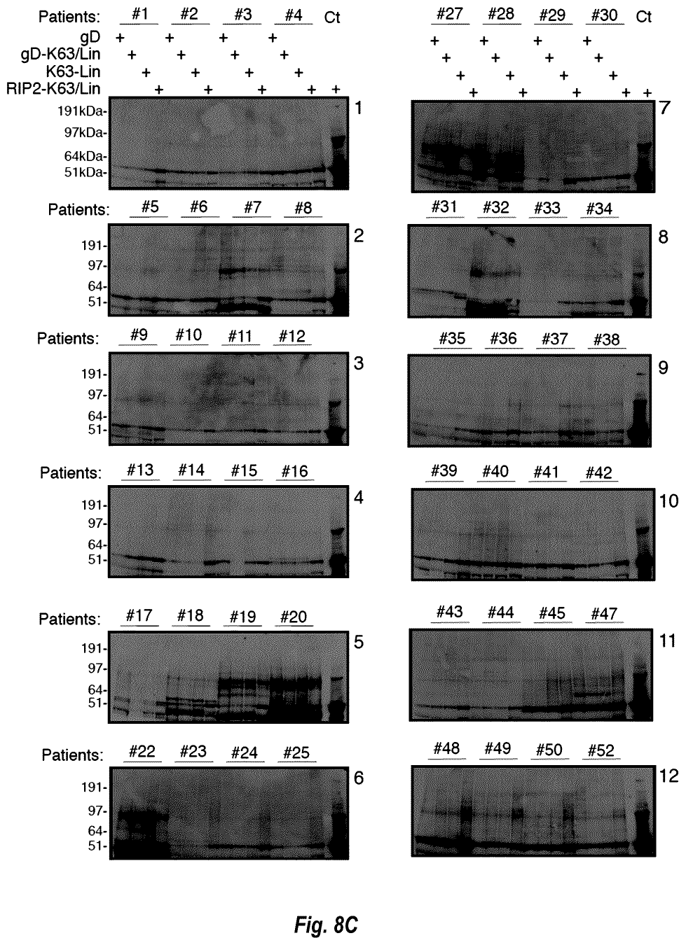

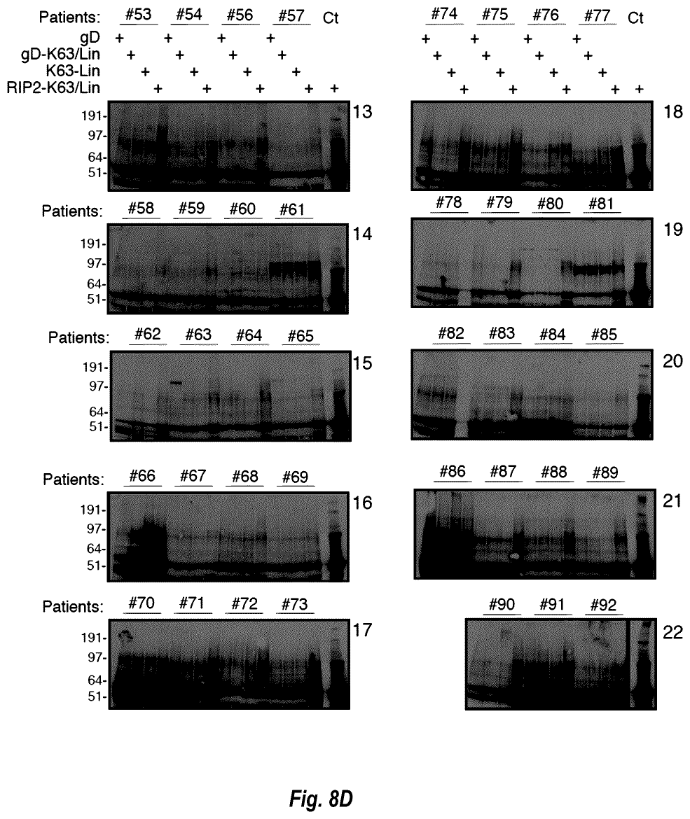

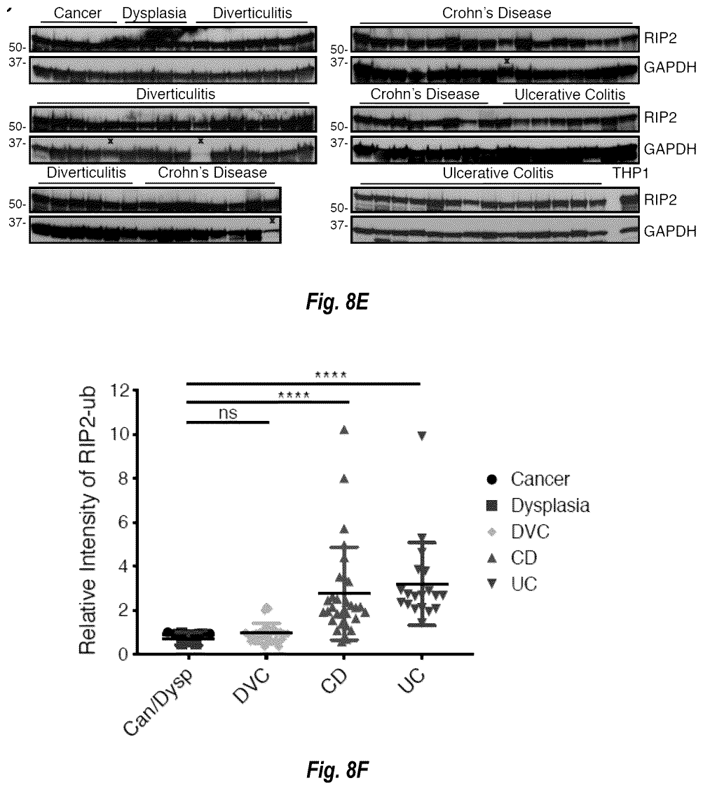

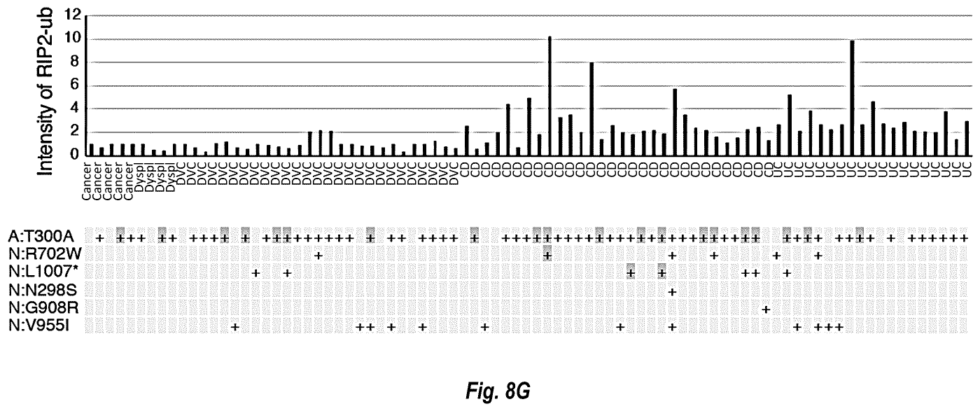

RIP2-K63 and RIP2-Lin bispecific antibodies were tested in intestinal tissue samples from patients (FIGS. 8A-G). Patients undergoing intestinal resection surgery for treatment of their colon cancer, dysplasia, diverticulitis (DVC), Crohn's disease (CD) or ulcerative colitis (UC) were enrolled in an observational study (Table 2).

TABLE 2
Clinical characteristics for IBD and non-IBD cohorts

| | Non-IBD subjects (n = 36) | | | |
|---|---|---|---|---|
| | Diverticulitis | Cancer/resected mass | UC patients | CD patients |
| | (n = 27) | (n = 9) | (n = 19) | (n = 30) |
| Age (median, range), | 59 | 68.5 | 56 | 44 |
| years | 37-75 | 50-89 | 17-72 | 18-83 |
| Sex (M/F/NR) | 10/16/1 | 5/4/0 | 13/6/0 | 12/17/1 |
| Previous biological | N/A | N/A | 11/8/0 | 21/7/2 |
| therapy (Y/N/NR) | | | | |

N/A, not applicable.
NR, not recorded.

Following surgery, intestinal tissue samples from patients with intestinal cancer, dysplasia, diverticulitis (DIV), Crohn's disease (CD) or ulcerative colitis (UC) were lysed in 6M urea lysis buffer and investigated by immunoprecipitated with the indicated bispecific antibodies.

FIG. 8A shows examples of immunoprecipitation from intestinal cancer, dysplasia, Crohn's disease or ulcerative colitis samples with the indicated bispecific antibodies. RIP2-K63 and RIP2-Lin bispecific antibodies immunoprecipitated ubiquitinated RIP2 with the strongest signal observed in CD and UC samples. FIGS. 8C and 8D show Samples 1-52 (FIG. 8C) and Samples 53-92 (FIG. 8D) from individual patients. The last lane in each panel contains immunoprecipitations with RIP2-K63 and RIP2-Lin combination of antibodies from MDP-treated (1 μg/ml, 30 min) THP1 cells and serves as a control (Ct). Tissue lysates from above listed patients prepared in 6 M urea lysis buffer were investigated by western blotting with RIP2 and GAPDH antibodies. Red asterisks indicate samples that were omitted from immunoprecipitations due to the poor quality and low protein levels. THP1 cell lysate serves as a control. This is shown in FIG. 8E.

Samples from patients with intestinal cancer, dysplasia, diverticulitis (DIV), Crohn's disease (CD) or ulcerative colitis (UC) were lysed in 6M urea lysis buffer and lysates immunoprecipitated with the combination of RIP2-K63 and RIP2-Lin bispecific antibodies. Western blotting was done with anti-RIP2 antibody, as shown in FIG. 8B. For better comparison of RIP2 ubiquitination signal between different samples, proteins immunoprecipitated by RIP2-K63 and RIP2-Lin bispecific antibodies were run on the gels next to each other, again demonstrating prominent RIP2 ubiquitination in CD and UC samples (FIG. 8B).

RIP2 ubiquitination in indicated patient samples was quantified by scanning western blots following immunoprecipitation with indicated antibodies as presented in FIGS. 8C and 8D. Scanned images were processed by ImageJ software and RIP2 ubiquitination was calculated as the ratio of gel intensity from immunoprecipitation with RIP2-K63/RIP2-Lin antibodies over immunoprecipitation with gD antibody, as shown in FIG. 8F. Ns indicates no significant difference, while three asterisks indicate p<0.0001. FIG. 8F also demonstrated a striking pattern of elevated RIP2 K63-linked and linear ubiquitination in CD and UC samples in comparison to intestinal cancer, dysplasia, and diverticulitis samples. Risk alleles for IBD have been identified using genome-wide association studies to identify common risk alleles for developing diseases (Liu et al., 2015). Two CD risk alleles, ATG16L1 and NOD2, that have been identified across populations, were further evaluated in this cohort with NOD2 being of particular interest as it is downstream of MDP.

This is shown in FIG. 8G, which provides RIP2 ubiquitination quantified in FIG. 8F presented in a bar graph format. Listed below the graph is genotype information for two common IBD risk loci: A stands for ATG16L1 and N for NOD2. Crohn's disease and ulcerative colitis patient samples were found to have elevated levels of RIP2 ubiquitination. Comparison of RIP2 ubiquitination levels with ATG16L1 and NOD2 risk alleles in FIG. 8F did not reveal any association suggesting that RIP2 ubiquitination in IBD samples is not correlated with these risk loci. Altogether, these data indicate that RIP2-K63 and RIP2-Lin bispecific antibodies can be used to investigate ubiquitination status of RIP2 in IBD samples.

J. Proteins Modified with K63-linked and Linear Ubiquitin Chains

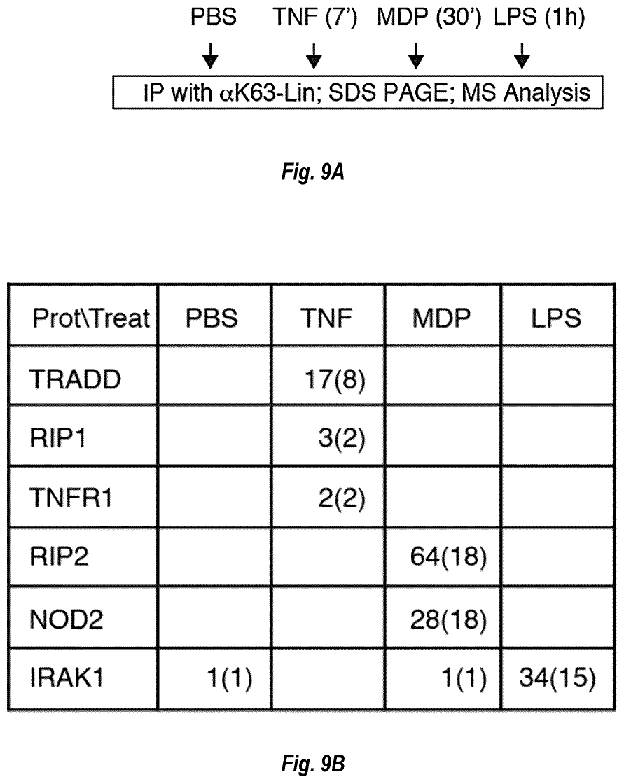

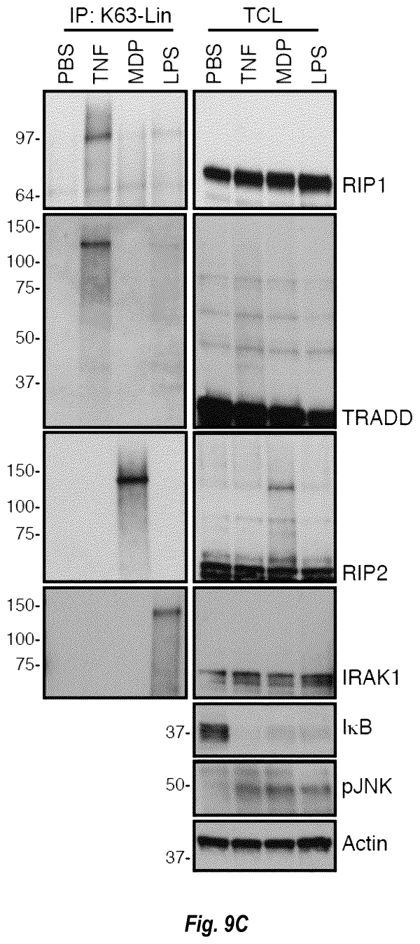

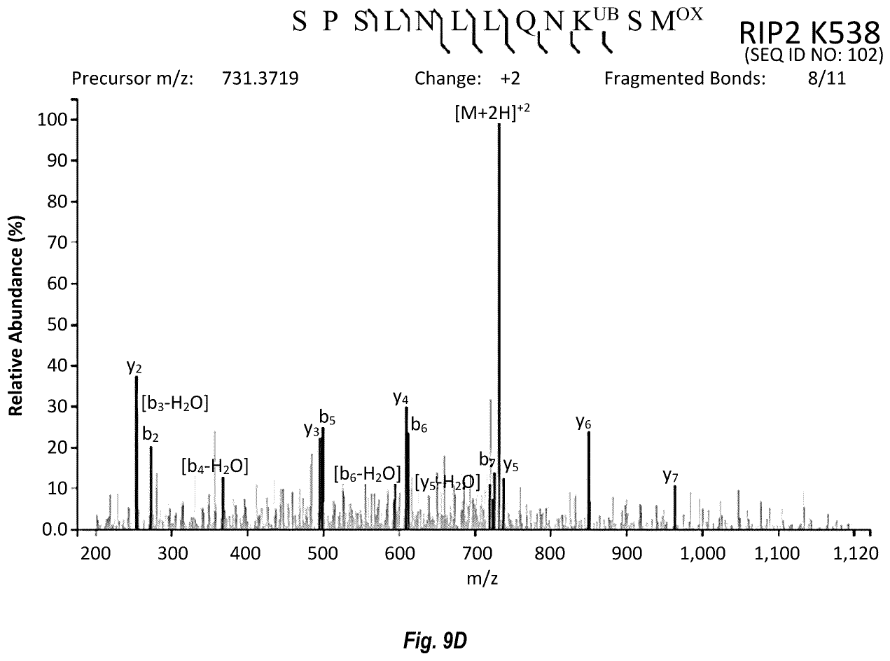

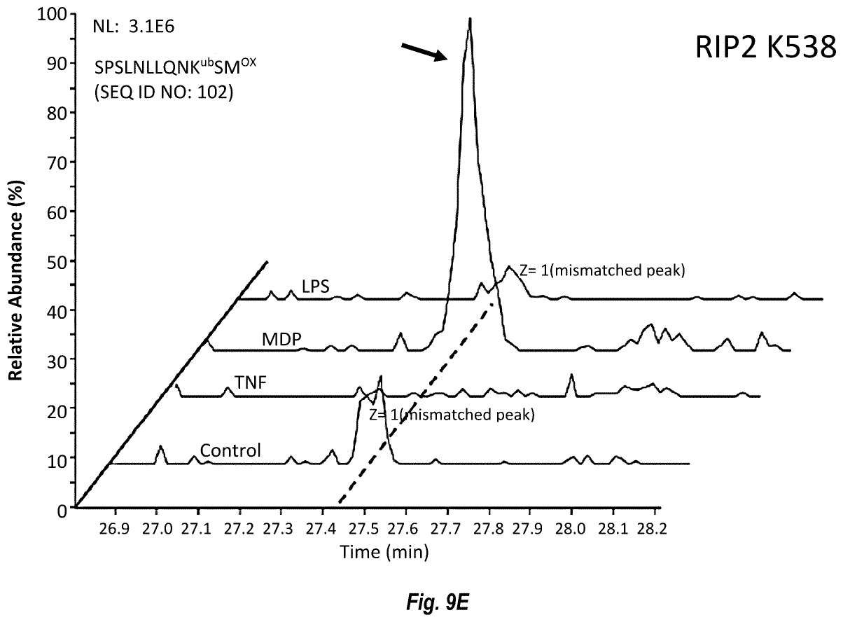

FIG. 9A provides a schematic of the experimental design scheme. THP1 cells were treated with PBS, TNF (100 ng/ml), MDP (1 μg/ml) or LPS (1 μg/ml) for indicated time periods, lysed in 6M urea lysis buffer, immunoprecipitated with K63-Lin bispecific antibody, and analyzed by mass spectroscopy.

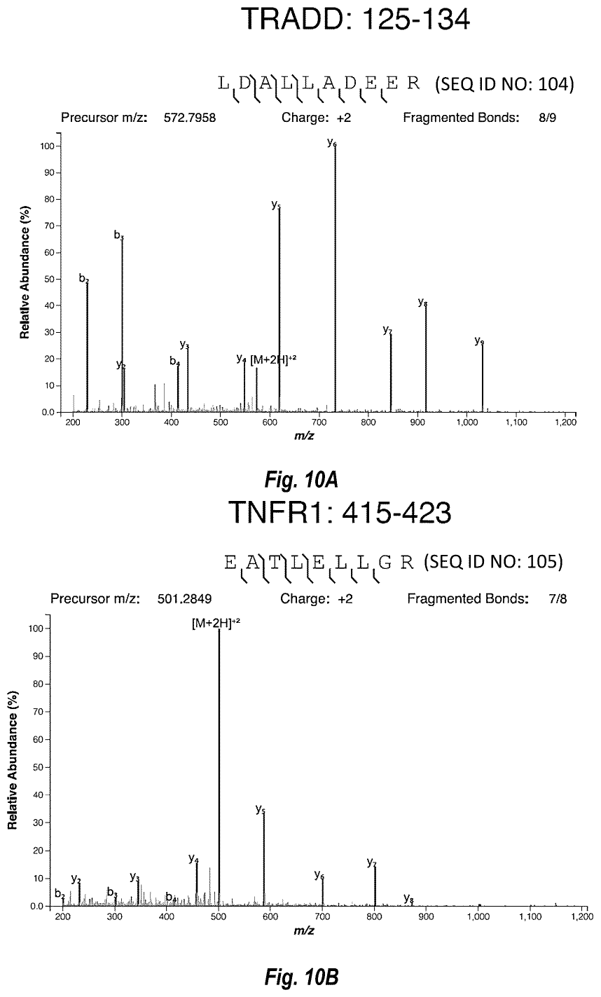

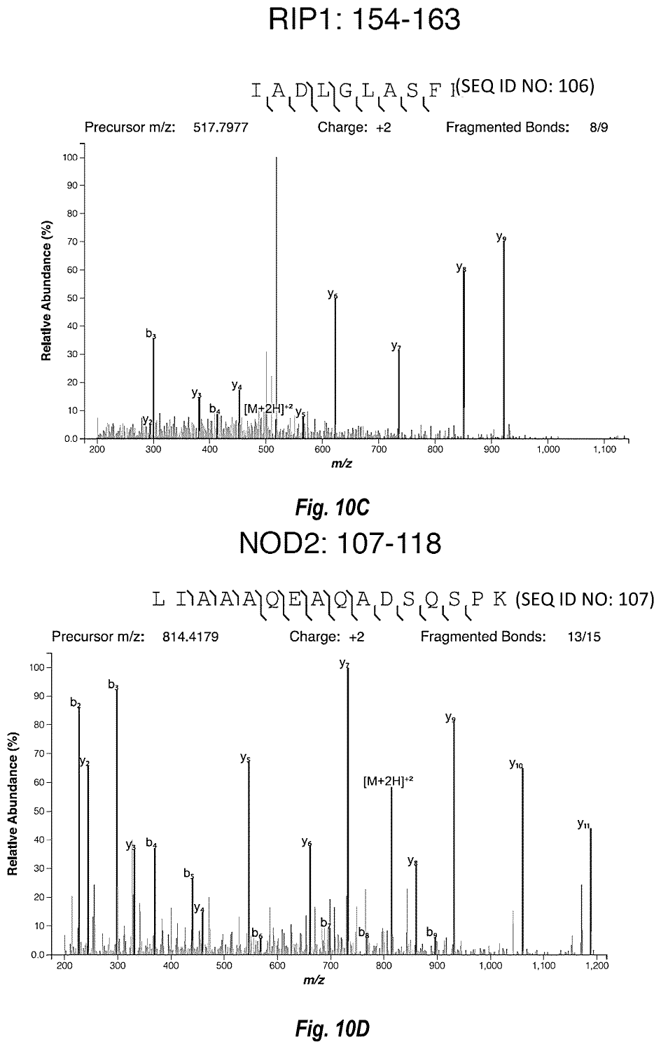

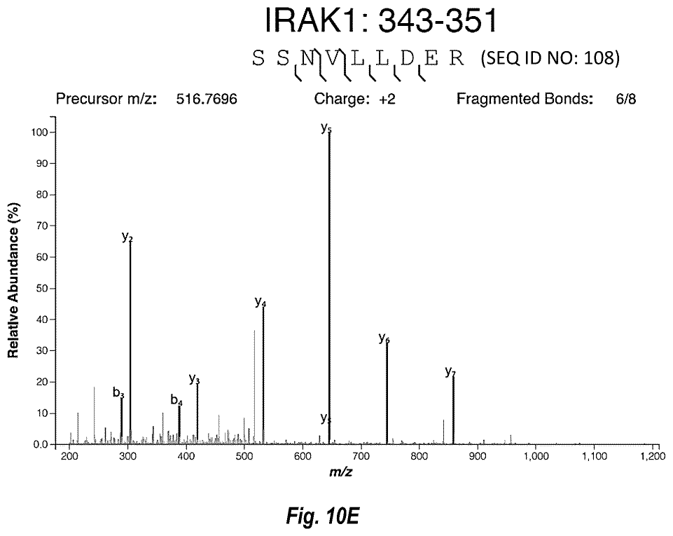

Immunoprecipitated proteins were separated on SDS-PAGE, excised and analyzed by mass spectrometry, which revealed distinct ubiquitination substrates dependent on stimulation. FIG. 9B provides the proteins identified by mass spectroscopy. Numbers indicate the total and unique (in parenthesis) number of identified peptides for each protein. TNF stimulation led to identification of known (RIP1, TNFR1) and novel (TRADD) ubiquitination substrates, while MDP and LPS treatments identified prominent K63-linked and linear ubiquitination of RIP2, NOD2 and IRAK1, respectively. This is also demonstrated in FIG. 10A-10E. The most robust identification signal from mass spectrometry analyses was for RIP2 following MDP treatment.

THP1 cells were treated with indicated stimuli as in FIG. 9A, lysed in 6M urea lysis buffer and immunoprecipitated with K63-Lin bispecific antibody. Immunoprecipitated proteins and total cell lysates proteins were probed with the indicated antibodies, as shown in FIG. 9C. This immunoprecipitation and western blot confirmed that proteins found to be ubiquitinated in mass spectrometry analyses were indeed modified by K63-linked and linear ubiquitination.

Selected ms/ms spectra matching to the RIP2 C-terminus SPSLNLLQNKSM (SEQ ID NO: 102) were annotated for matched fragment ions (FIG. 9D) and their extracted ion chromatography (XIC) from four treatment conditions are illustrated. Matched XIC peaks are highlighted by the arrow (FIG. 9E), to identify ubiquitinated RIP2 from the MDP-treated condition. K^{ub} indicates ubiquitination at RIP2 position K538. M^{ox} indicates oxidation at position M540 of the RIP2 polypeptide. This showed that K538 is the site of predominant RIP2 modification with K63-linked and/or linear ubiquitination. Together, these show that K63-Lin bispecific antibody can be used to identify the specific ubiquitination site, in addition to the general protein modification.

This demonstrates that K63-Lin bispecific antibody can be used to detect and identify proteins ubiquitinated with K63-linked and linear chains in different signaling pathways.

Although the foregoing invention has been described in some detail by way of illustration and example for purposes of clarity of understanding, the descriptions and examples should not be construed as limiting the scope of the invention.

TABLE OF
SEQUENCES

| SEQ | | |
|---|---|---|
| ID | | |
| NO | Description | Sequence |
| 1 | anti-K11 LC (with signal | MGWSCIILFLVATATGVHSDIQMTQSPSSLSASVGDRVTITCRASQI |
| | sequence underlined) | VGTFVAWYQQKPGKAPKLLIYSASFLYSGVPSRFSGSGSGTDFTLTI |
| | | SSLQPEDFATYYCQQSYTTPPTFGQGTKVEIKRTVAAPSVFIFPPSD |
| | | EQLKSGTASVVCLLNNFYPREAKVQWKVDNALQSGNSQESVTEQDSK |
| | | DSTYSLSSTLTLSKADYEKHKVYACEVTHQGLSSPVTKSFNRGEC |
| 2 | anti-K11 mature LC | DIQMTQSPSSLSASVGDRVTITCRASQIVGTFVAWYQQKPGKAPKLL |
| | | IYSASFLYSGVPSRFSGSGSGTDFTLTISSLQPEDFATYYCQQSYTT |
| | | PPTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 3 | anti-K11 knob HC (with | MGWSCIILFLVATATGAYAEVQLVESGGGLVQPGGSLRLSCAASGFT |
| | signal sequence | FSNSYISWVRQAPGKGLEWVAAINPAGGYTYYADSVKGRFTISADTS |
| | underlined) | KNTAYLQMNSLRAEDTAVYYCAREWYFGGYVMDYWGQGTLVTVSSAS |
| | | TKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLWCLVKGFYPSDIAVEWESNGQPENNYKTTPPVLDSD |
| | | GSFFLYSKLTVDKSRWQQGNVFSCSVMHEALHNHYTQKSLSLSPGK |
| 4 | anti-K11 mature knob HC | EVQLVESGGGLVQPGGSLRLSCAASGFTFSNSYISWVRQAPGKGLEW |
| | | VAAINPAGGYTYYADSVKGRFTISADTSKNTAYLQMNSLRAEDTAVY |
| | | YCAREWYFGGYVMDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNK |
| | | ALPAPIEKTISKAKGQPREPQVYTLPPSREEMTKNQVSLWCLVKGFY |
| | | PSDIAVEWESNGQPENNYKTTPPVLDSDGSFFLYSKLTVDKSRWQQG |
| | | NVFSCSVMHEALHNHYTQKSLSLSPGK |
| 5 | anti-K11 hole HC (with | MGWSCIILFLVATATGAYAEVQLVESGGGLVQPGGSLRLSCAASGFT |
| | signal sequence | FSNSYISWVRQAPGKGLEWVAAINPAGGYTYYADSVKGRFTISADTS |
| | underlined) | KNTAYLQMNSLRAEDTAVYYCAREWYFGGYVMDYWGQGTLVTVSSAS |
| | | TKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSPGK |
| 6 | anti-K11 mature hole HC | EVQLVESGGGLVQPGGSLRLSCAASGFTFSNSYISWVRQAPGKGLEW |
| | | VAAINPAGGYTYYADSVKGRFTISADTSKNTAYLQMNSLRAEDTAVY |
| | | YCAREWYFGGYVMDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNK |
| | | ALPAPIEKTISKAKGQPREPQVYTLPPSREEMTKNQVSLSCAVKGFY |
| | | PSDIAVEWESNGQPENNYKTTPPVLDSDGSFFLVSKLTVDKSRWQQG |
| | | NVFSCSVMHEALHNHYTQKSLSLSPGK |
| 7 | anti-K11 LCVR (HVRs | DIQMTQSPSSLSASVGDRVTITCRASQIVGTFVAWYQQKPGKAPKLL |
| | underlined) | IYSASFLYSGVPSRFSGSGSGTDFTLTISSLQPEDFATYYCQQSYTT |
| | | PPTFGQGTKVEIK |
| 8 | anti-K11 HCVR (HVRs | EVQLVESGGGLVQPGGSLRLSCAASGFTFSNSYISWVRQAPGKGLEW |
| | underlined) | VAAINPAGGYTYYADSVKGRFTISADTSKNTAYLQMNSLRAEDTAVY |
| | | YCAREWYFGGYVMDYWGQGTLVTVSS |
| 9 | anti-K11 HVR-L1 | RASQIVGTFVA |
| 10 | anti-K11 HVR-L2 | SASFLYS |
| 11 | anti-K11 HVR-L3 | QQSYTTPPT |
| 12 | anti-K11 HVR-H1 | GFTFSNSYIS |
| 13 | anti-K11 HVR-H2 | AINPAGGYTYYADSVKG |
| 14 | anti-K11 HVR-H3 | AREWYFGGYVMDY |
| 15 | anti-K48 LC (with signal | MGWSCIILFLVATATGVHSDIQMTQSPSSLSASVGDRVTITCRASQS |
| | sequence underlined) | VSSAVAWYQQKPGKAPKLLIYSASSLYSGVPSRFSGSRSGTDFTLTI |
| | | SSLQPEDFATYYCQQSSYSSLITFGQGTKVEIKRTVAAPSVFIFPPS |
| | | DEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 16 | anti-K48 mature LC | DIQMTQSPSSLSASVGDRVTITCRASQSVSSAVAWYQQKPGKAPKLL |
| | | IYSASSLYSGVPSRFSGSRSGTDFTLTISSLQPEDFATYYCQQSSYS |
| | | SLITFGQGTKVEIKRTVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEC |
| 17 | anti-K48 knob HC (with | MGWSCIILFLVATATGAYAEVQLVESGGGLVQPGGSLRLSCAASGEN |
| | signal sequence | ISYSSMHWVRQAPGKGLEWVASIYSYYSYTSYADSVKGRFTISADTS |
| | underlined) | KNTAYLQMNSLRAEDTAVYYCARSYSYHLGMDYWGQGTLVTVSSAST |
| | | KGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLWCLVKGFYPSDIAVEWESNGQPENNYKTTPPVLDSDG |
| | | SFFLYSKLTVDKSRWQQGNVFSCSVMHEALHNHYTQKSLSLSPGK |
| 18 | anti-K48 mature knob HC | EVQLVESGGGLVQPGGSLRLSCAASGENISYSSMHWVRQAPGKGLEW |
| | | VASIYSYYSYTSYADSVKGRFTISADTSKNTAYLQMNSLRAEDTAVY |
| | | YCARSYSYHLGMDYWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSREEMTKNQVSLWCLVKGFYP |
| | | SDIAVEWESNGQPENNYKTTPPVLDSDGSFFLYSKLTVDKSRWQQGN |
| | | VFSCSVMHEALHNHYTQKSLSLSPGK |
| 19 | anti-K48 hole HC (with | MGWSCIILFLVATATGAYAEVQLVESGGGLVQPGGSLRLSCAASGEN |
| | signal sequence | ISYSSMHWVRQAPGKGLEWVASIYSYYSYTSYADSVKGRFTISADTS |
| | underlined) | KNTAYLQMNSLRAEDTAVYYCARSYSYHLGMDYWGQGTLVTVSSAST |
| | | KGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLSCAVKGFYPSDIAVEWESNGQPENNYKTTPPVLDSDG |
| | | SFFLVSKLTVDKSRWQQGNVFSCSVMHEALHNHYTQKSLSLSPGK |
| 20 | anti-K48 mature hole HC | EVQLVESGGGLVQPGGSLRLSCAASGENISYSSMHWVRQAPGKGLEW |
| | | VASIYSYYSYTSYADSVKGRFTISADTSKNTAYLQMNSLRAEDTAVY |
| | | YCARSYSYHLGMDYWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSREEMTKNQVSLSCAVKGFYP |
| | | SDIAVEWESNGQPENNYKTTPPVLDSDGSFFLVSKLTVDKSRWQQGN |
| | | VFSCSVMHEALHNHYTQKSLSLSPGK |
| 21 | anti-K48 LCVR (HVRs | DIQMTQSPSSLSASVGDRVTITCRASQSVSSAVAWYQQKPGKAPKLL |
| | underlined) | IYSASSLYSGVPSRFSGSRSGTDFTLTISSLQPEDFATYYCQQSSYS |
| | | SLITFGQGTKVEIK |
| 22 | anti-K48 HCVR (HVRs | EVQLVESGGGLVQPGGSLRLSCAASGFNISYSSMHWVRQAPGKGLEW |
| | underlined) | VASIYSYYSYTSYADSVKGRFTISADTSKNTAYLQMNSLRAEDTAVY |
| | | YCARSYSYHLGMDYWGQGTLVTVSS |
| 23 | anti-K48 HVR-L1 | RASQSVSSAVA |
| 24 | anti-K48 HVR-L2 | SASSLYS |
| 25 | anti-K48 HVR-L3 | QQSSYSSLIT |
| 26 | anti-K48 HVR-H1 | GFNISYSSMH |
| 27 | anti-K48 HVR-H2 | SIYSYYSYTSYADSVKG |
| 28 | anti-K48 HVR-H3 | ARSYSYHLGMDY |
| 29 | anti-K63 LC (with signal | MGWSCIILFLVATATGVHSDIQMTQSPSSLSASVGDRVTITCRASQS |
| | sequence underlined) | VSSAVAWYQQKPGEAPKLLIYSARSLYSGVPSRFSGSRSGTDFTLTI |
| | | SSLQPEDFATYYCQQYSSYSSLFTFGQGTKVEIKRTVAAPSVFIFPP |
| | | SDEQLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 30 | anti-K63 mature LC | DIQMTQSPSSLSASVGDRVTITCRASQSVSSAVAWYQQKPGEAPKLL |
| | | IYSARSLYSGVPSRFSGSRSGTDFTLTISSLQPEDFATYYCQQYSSY |
| | | SSLFTFGQGTKVEIKRTVAAPSVFIFPPSDEQLKSGTASVVCLLNNF |
| | | YPREAKVQWKVDNALQSGNSQESVTEQDSKDSTYSLSSTLTLSKADY |
| | | EKHKVYACEVTHQGLSSPVTKSFNRGEC |
| 31 | anti-K63 knob HC (with | MGWSCIILFLVATATGAYAEVQLVESGGGLVQPGGSLRLSCAASGEN |
| | signal sequence | VKTGLIHWVRQAPGKGLEWVAYITPYYGSTSYADSVKGRFTISADTS |
| | underlined) | KNTAYLQMNSLRAEDTAVYYCAREYYRWYTAIDYWGQGTLVTVSSAS |
| | | TKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLWCLVKGFYPSDIAVEWESNGQPENNYKTTPPVLDSD |
| | | GSFFLYSKLTVDKSRWQQGNVFSCSVMHEALHNHYTQKSLSLSPGK |
| 32 | anti-K63 mature knob HC | EVQLVESGGGLVQPGGSLRLSCAASGFNVKTGLIHWVRQAPGKGLEW |
| | | VAYITPYYGSTSYADSVKGRFTISADTSKNTAYLQMNSLRAEDTAVY |
| | | YCAREYYRWYTAIDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNK |
| | | ALPAPIEKTISKAKGQPREPQVYTLPPSREEMTKNQVSLWCLVKGFY |
| | | PSDIAVEWESNGQPENNYKTTPPVLDSDGSFFLYSKLTVDKSRWQQG |
| | | NVFSCSVMHEALHNHYTQKSLSLSPGK |
| 33 | anti-K63 hole HC (with | MGWSCIILFLVATATGAYAEVQLVESGGGLVQPGGSLRLSCAASGEN |
| | signal sequence | VKTGLIHWVRQAPGKGLEWVAYITPYYGSTSYADSVKGRFTISADTS |
| | underlined) | KNTAYLQMNSLRAEDTAVYYCAREYYRWYTAIDYWGQGTLVTVSSAS |
| | | TKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSPGK |
| 34 | anti-K63 mature hole HC | EVQLVESGGGLVQPGGSLRLSCAASGENVKTGLIHWVRQAPGKGLEW |
| | | VAYITPYYGSTSYADSVKGRFTISADTSKNTAYLQMNSLRAEDTAVY |
| | | YCAREYYRWYTAIDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNK |
| | | ALPAPIEKTISKAKGQPREPQVYTLPPSREEMTKNQVSLSCAVKGFY |
| | | PSDIAVEWESNGQPENNYKTTPPVLDSDGSFFLVSKLTVDKSRWQQG |
| | | NVFSCSVMHEALHNHYTQKSLSLSPGK |
| 35 | anti-K63 LCVR (HVRs | DIQMTQSPSSLSASVGDRVTITCRASQSVSSAVAWYQQKPGEAPKLL |
| | underlined) | IYSARSLYSGVPSRFSGSRSGTDFTLTISSLQPEDFATYYCQQYSSY |
| | | SSLFTFGQGTKVEIK |
| 36 | anti-K63 HCVR (HVRs | EVQLVESGGGLVQPGGSLRLSCAASGFNVKTGLIHWVRQAPGKGLEW |
| | underlined) | VAYITPYYGSTSYADSVKGRFTISADTSKNTAYLQMNSLRAEDTAVY |
| | | YCAREYYRWYTAIDYWGQGTLVTVSS |
| 37 | anti-K63 HVR-L1 | RASQSVSSAVA |
| 38 | anti-K63 HVR-L2 | SARSLYS |
| 39 | anti-K63 HVR-L3 | QQYSSYSSLFT |
| 40 | anti-K63 HVR-H1 | GFNVKTGLIH |
| 41 | anti-K63 HVR-H2 | YITPYYGSTSYADSVKG |
| 42 | anti-K63 HVR-H3 | AREYYRWYTAIDY |
| 43 | anti-M1 LC (with signal | MGWSCIILFLVATATGVHSDIQMTQSPSSLSASVGDRVTITCRASQD |
| | sequence underlined) | VSTAVAWYQQKPGKAPKLLIYSAKFLYSGVPSRFSGSGSGTDFTLTI |
| | | SSLQPEDFATYYCQQSYTTPPTFGQGTKVEIKRTVAAPSVFIFPPSD |
| | | EQLKSGTASVVCLLNNFYPREAKVQWKVDNALQSGNSQESVTEQDSK |
| | | DSTYSLSSTLTLSKADYEKHKVYACEVTHQGLSSPVTKSFNRGEC |
| 44 | anti-M1 mature LC | DIQMTQSPSSLSASVGDRVTITCRASQDVSTAVAWYQQKPGKAPKLL |
| | | IYSAKFLYSGVPSRFSGSGSGTDFTLTISSLQPEDFATYYCQQSYTT |
| | | PPTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 45 | anti-M1 knob HC (with | MGWSCIILFLVATATGAYAEVQLVESGGGLVQPGGSLRLSCAASGFT |
| | signal sequence | FSNTYISWVRQAPGKGLEWVASITPSSGQTDYADSVKGRFTISADTS |
| | underlined) | KNTAYLQMNSLRAEDTAVYYCARTWLLRWVMDLWGQGTLVTVSSAST |
| | | KGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLWCLVKGFYPSDIAVEWESNGQPENNYKTTPPVLDSDG |
| | | SFFLYSKLTVDKSRWQQGNVFSCSVMHEALHNHYTQKSLSLSPGK |
| 46 | anti-M1 mature knob HC | EVQLVESGGGLVQPGGSLRLSCAASGFTFSNTYISWVRQAPGKGLEW |
| | | VASITPSSGQTDYADSVKGRFTISADTSKNTAYLQMNSLRAEDTAVY |
| | | YCARTWLLRWVMDLWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSREEMTKNQVSLWCLVKGFYP |
| | | SDIAVEWESNGQPENNYKTTPPVLDSDGSFFLYSKLTVDKSRWQQGN |
| | | VFSCSVMHEALHNHYTQKSLSLSPGK |
| 47 | anti-M1 hole HC (with | MGWSCIILFLVATATGAYAEVQLVESGGGLVQPGGSLRLSCAASGFT |
| | signal sequence | FSNTYISWVRQAPGKGLEWVASITPSSGQTDYADSVKGRFTISADTS |
| | underlined) | KNTAYLQMNSLRAEDTAVYYCARTWLLRWVMDLWGQGTLVTVSSAST |
| | | KGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLSCAVKGFYPSDIAVEWESNGQPENNYKTTPPVLDSDG |
| | | SFFLVSKLTVDKSRWQQGNVFSCSVMHEALHNHYTQKSLSLSPGK |
| 48 | anti-M1 mature hole HC | EVQLVESGGGLVQPGGSLRLSCAASGFTFSNTYISWVRQAPGKGLEW |
| | | VASITPSSGQTDYADSVKGRFTISADTSKNTAYLQMNSLRAEDTAVY |
| | | YCARTWLLRWVMDLWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSREEMTKNQVSLSCAVKGFYP |
| | | SDIAVEWESNGQPENNYKTTPPVLDSDGSFFLVSKLTVDKSRWQQGN |
| | | VFSCSVMHEALHNHYTQKSLSLSPGK |
| 49 | anti-M1 LCVR (HVRs | DIQMTQSPSSLSASVGDRVTITCRASQDVSTAVAWYQQKPGKAPKLL |
| | underlined) | IYSAKFLYSGVPSRFSGSGSGTDFTLTISSLQPEDFATYYCQQSYTT |
| | | PPTFGQGTKVEIK |
| 50 | anti-M1 HCVR (HVRs | EVQLVESGGGLVQPGGSLRLSCAASGFTFSNTYISWVRQAPGKGLEW |
| | underlined) | VASITPSSGQTDYADSVKGRFTISADTSKNTAYLQMNSLRAEDTAVY |
| | | YCARTWLLRWVMDLWGQGTLVTVSS |
| 51 | anti-M1 HVR-L1 | RASQDVSTAVA |
| 52 | anti-M1 HVR-L2 | SAKFLYS |
| 53 | anti-M1 HVR-L3 | QQSYTTPPT |
| 54 | anti-M1 HVR-H1 | GFTFSNTYIS |
| 55 | anti-M1 HVR-H2 | SITPSSGQTDYADSVKG |
| 56 | anti-M1 HVR-H3 | ARTWLLRWVMDL |
| 57 | anti-RIP1 LC1 (with | MGWSCIILFLVATATGVHSNIMMTQSPSSLAVSAGQKVTMNCKSSQN |
| | signal sequence | VIYTSNQRNYLAWYQQKPGQSPKLLIYWASTRVSGVPDRFTGSGSGT |
| | underlined) | DFTLTISSVEVEDLAVYYCHQYLSSWTFGGGTKLEIKRADAAPTVSI |
| | | FPPSSEQLTSGGASVVCFLNNFYPKDINVKWKIDGSERQNGVLNSWT |
| | | DQDSKDSTYSMSSTLTLTKDEYERHNSYTCEATHKTSTSPIVKSFNR |
| | | NEC |
| 58 | anti-RIP1 mature LC1 | NIMMTQSPSSLAVSAGQKVTMNCKSSQNVIYTSNQRNYLAWYQQKPG |
| | (from de novo | QSPKLLIYWASTRVSGVPDRFTGSGSGTDFTLTISSVEVEDLAVYYC |
| | sequencing) | HQYLSSWTFGGGTKLEIKRADAAPTVSIFPPSSEQLTSGGASVVCFL |
| | | NNFYPKDINVKWKIDGSERQNGVLNSWTDQDSKDSTYSMSSTLTLTK |
| | | DEYERHNSYTCEATHKTSTSPIVKSFNRNEC |
| 59 | anti-RIP1 LC1 (with | MGWSCIILFLVATATGVHSNIMMTQSPSSLAVSAGQKVTMNCKSSQN |
| | signal sequence | VIYTSNQRNYLAWYQQKPGQSPKLLIYWASTRVSGVPDRFTGSGSGT |
| | underlined) having one | DFTLTISSVEVEDLAVYYCHQYLSSWTFGGGTKVEIKRTVAAPSVFI |
| | amino acid difference | FPPSDEQLKSGTASVVCLLNNFYPREAKVQWKVDNALQSGNSQESVT |
| | L104V | EQDSKDSTYSLSSTLTLSKADYEKHKVYACEVTHQGLSSPVTKSFNR |
| | | GEC |
| 60 | anti-RIP1 mature LC1 | NIMMTQSPSSLAVSAGQKVTMNCKSSQNVIYTSNQRNYLAWYQQKPG |
| | having one amino acid | QSPKLLIYWASTRVSGVPDRFTGSGSGTDFTLTISSVEVEDLAVYYC |
| | difference L104V | HQYLSSWTFGGGTKVEIKRTVAAPSVFIFPPSDEQLKSGTASVVCLL |
| | | NNFYPREAKVQWKVDNALQSGNSQESVTEQDSKDSTYSLSSTLTLSK |
| | | ADYEKHKVYACEVTHQGLSSPVTKSFNRGEC |
| 61 | anti-RIP1 LC2 (with | MGWSCIILFLVATATGVHSNIMMTQSPSSLAVSAGQKVTMNCKSSQN |
| | signal sequence | VIYTSNQRNYLAWYQQKPGQSPKLLIYGASNRYTGVPDRFTGSGSGT |
| | underlined) | DFTLTISSVEVEDLAVYYCHQYLSSWTFGGGTKLEIKRADAAPTVSI |
| | | FPPSSEQLTSGGASVVCFLNNFYPKDINVKWKIDGSERQNGVLNSWT |
| | | DQDSKDSTYSMSSTLTLTKDEYERHNSYTCEATHKTSTSPIVKSFNR |
| | | NEC |
| 62 | anti-RIP1 mature LC2 | NIMMTQSPSSLAVSAGQKVTMNCKSSQNVIYTSNQRNYLAWYQQKPG |
| | | QSPKLLIYGASNRYTGVPDRFTGSGSGTDFTLTISSVEVEDLAVYYC |
| | | HQYLSSWTFGGGTKLEIKRADAAPTVSIFPPSSEQLTSGGASVVCFL |
| | | NNFYPKDINVKWKIDGSERQNGVLNSWTDQDSKDSTYSMSSTLTLTK |
| | | DEYERHNSYTCEATHKTSTSPIVKSFNRNEC |
| 63 | anti-RIP1 HC (with signal | MGWSCIILFLVATATGAYADVQLVESGGGLVQPGGSRKLSCAASGFT |
| | sequence underlined) | FGSFGMHWVRQTPEKRLEWIAYISSGGSTIYYADTVKGRFTISRDTP |
| | | KNTLFLQMTSLRSEDTAMYFCARSIMISTEFDYWGQGTTLTVSSAKT |
| | | TAPSVYPLAPVCGDTTGSSVTLGCLVKGYFPEPVTLTWNSGSLSSGV |
| | | HTFPAVLQSDLYTLSSSVTVTSSTWPSQSITCNVAHPASSTKVDKKI |
| | | EPRGPTIKPCPPCKCPAPNLLGGPSVFIFPPKIKDVLMISLSPIVTC |
| | | VVVDVSEDDPDVQISWFVNNVEVHTAQTQTHREDYNSTLRVVSALPI |
| | | QHQDWMSGKEFKCKVNNKDLPAPIERTISKPKGSVRAPQVYVLPPPE |
| | | EEMTKKQVTLTCMVTDFMPEDIYVEWTDNGKTELNYKNTEPVLDSDG |
| | | SYFMYSKLRVEKKNWVERNSYSCSVVHEGLHNHHTTKSFSRTPG |
| 64 | anti-RIP1 mature HC | DVQLVESGGGLVQPGGSRKLSCAASGFTFGSFGMHWVRQTPEKRLEW |
| | (from de novo | IAYISSGGSTIYYADTVKGRFTISRDTPKNTLFLQMTSLRSEDTAMY |
| | sequencing) | FCARSIMISTEFDYWGQGTTLTVSSAKTTAPSVYPLAPVCGDTTGSS |
| | | VTLGCLVKGYFPEPVTLTWNSGSLSSGVHTFPAVLQSDLYTLSSSVT |
| | | VTSSTWPSQSITCNVAHPASSTKVDKKIEPRGPTIKPCPPCKCPAPN |
| | | LLGGPSVFIFPPKIKDVLMISLSPIVTCVVVDVSEDDPDVQISWFVN |
| | | NVEVHTAQTQTHREDYNSTLRVVSALPIQHQDWMSGKEFKCKVNNKD |
| | | LPAPIERTISKPKGSVRAPQVYVLPPPEEEMTKKQVTLTCMVTDEMP |
| | | EDIYVEWTDNGKTELNYKNTEPVLDSDGSYFMYSKLRVEKKNWVERN |
| | | SYSCSVVHEGLHNHHTTKSFSRTPG |
| 65 | anti-RIP1 HC (with signal | MGWSCIILFLVATATGAYADVQLVESGGGLVQPGGSRKLSCAASGFT |
| | sequence underlined) | FGSFGMHWVRQTPEKRLEWIAYISSGGSTIYYADTVKGRFTISRDTP |
| | having two amino acid | KNTLFLQMTSLRSEDTAMYFCARSIMISTEFDYWGQGTTLTVSSAKT |
| | differences in constant | TGPSVYPLAPVCGDTTGSSVTLGCLVKGYFPEPVTLTWNSGSLSSGV |
| | region | HTFPAVLQSDLYTLSSSVTVTSSTWPSQSITCNVAHPASSTKVDKKI |
| | | EPRGPTIKPCPPCKCPAPNLLGGPSVFIFPPKIKDVLMISLSPIVTC |
| | | VVVDVSEDDPDVQISWFVNNVEVHTAQTQTHREDYNSTLRVVSALPI |
| | | QHQDWMSGKEFKCKVNNKDLPAPIERTISKPKGSVRAPQVYVLPPPE |
| | | EEMTKKQVTLTCMVTDFMPEDIYVEWTNNGKTELNYKNTEPVLDSDG |
| | | SYFMYSKLRVEKKNWVERNSYSCSVVHEGLHNHHTTKSFSRTPGK |
| 66 | anti-RIP1 mature HC | DVQLVESGGGLVQPGGSRKLSCAASGFTFGSFGMHWVRQTPEKRLEW |
| | having two amino acid | IAYISSGGSTIYYADTVKGRFTISRDTPKNTLFLQMTSLRSEDTAMY |
| | differences in constant | FCARSIMISTEFDYWGQGTTLTVSSAKTTGPSVYPLAPVCGDTTGSS |
| | region | VTLGCLVKGYFPEPVTLTWNSGSLSSGVHTFPAVLQSDLYTLSSSVT |
| | | VTSSTWPSQSITCNVAHPASSTKVDKKIEPRGPTIKPCPPCKCPAPN |
| | | LLGGPSVFIFPPKIKDVLMISLSPIVTCVVVDVSEDDPDVQISWFVN |
| | | NVEVHTAQTQTHREDYNSTLRVVSALPIQHQDWMSGKEFKCKVNNKD |
| | | LPAPIERTISKPKGSVRAPQVYVLPPPEEEMTKKQVTLTCMVTDFMP |
| | | EDIYVEWTNNGKTELNYKNTEPVLDSDGSYFMYSKLRVEKKNWVERN |
| | | SYSCSVVHEGLHNHHTTKSFSRTPGK |
| 67 | anti-RIP1 knob HC (with | MGWSCIILFLVATATGAYADVQLVESGGGLVQPGGSRKLSCAASGFT |
| | signal sequence | FGSFGMHWVRQTPEKRLEWIAYISSGGSTIYYADTVKGRFTISRDTP |
| | underlined) | KNTLFLQMTSLRSEDTAMYFCARSIMISTEFDYWGQGTTLTVSSAKT |
| | | TGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLWCLVKGFYPSDIAVEWESNGQPENNYKTTPPVLDSDG |
| | | SFFLYSKLTVDKSRWQQGNVFSCSVMHEALHNHYTQKSLSLSPGK |
| 68 | anti-RIP1 mature knob | DVQLVESGGGLVQPGGSRKLSCAASGFTFGSFGMHWVRQTPEKRLEW |
| | HC | IAYISSGGSTIYYADTVKGRFTISRDTPKNTLFLQMTSLRSEDTAMY |
| | | FCARSIMISTEFDYWGQGTTLTVSSAKTTGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSREEMTKNQVSLWCLVKGFYP |
| | | SDIAVEWESNGQPENNYKTTPPVLDSDGSFFLYSKLTVDKSRWQQGN |
| | | VFSCSVMHEALHNHYTQKSLSLSPGK |
| 69 | anti-RIP1 hole HC (with | MGWSCIILFLVATATGAYADVQLVESGGGLVQPGGSRKLSCAASGFT |
| | signal sequence | FGSFGMHWVRQTPEKRLEWIAYISSGGSTIYYADTVKGRFTISRDTP |
| | underlined) | KNTLFLQMTSLRSEDTAMYFCARSIMISTEFDYWGQGTTLTVSSAKT |
| | | TGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLSCAVKGFYPSDIAVEWESNGQPENNYKTTPPVLDSDG |
| | | SFFLVSKLTVDKSRWQQGNVFSCSVMHEALHNHYTQKSLSLSPGK |
| 70 | anti-RIP1 mature hole HC | DVQLVESGGGLVQPGGSRKLSCAASGFTFGSFGMHWVRQTPEKRLEW |
| | | IAYISSGGSTIYYADTVKGRFTISRDTPKNTLFLQMTSLRSEDTAMY |
| | | FCARSIMISTEFDYWGQGTTLTVSSAKTTGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSREEMTKNQVSLSCAVKGFYP |
| | | SDIAVEWESNGQPENNYKTTPPVLDSDGSFFLVSKLTVDKSRWQQGN |
| | | VFSCSVMHEALHNHYTQKSLSLSPGK |
| 71 | anti-RIP1 LC1 LCVR | NIMMTQSPSSLAVSAGQKVTMNCKSSQNVIYTSNQRNYLAWYQQKPG |
| | (HVRs underlined) having | QSPKLLIYWASTRVSGVPDRFTGSGSGTDFTLTISSVEVEDLAVYYC |
| | one amino acid difference | HQYLSSWTFGGGTKVEIK |
| | L104V | |
| 72 | anti-RIP1 LC2 LCVR | NIMMTQSPSSLAVSAGQKVTMNCKSSQNVIYTSNQRNYLAWYQQKPG |
| | (HVRs underlined) | QSPKLLIYGASNRYTGVPDRFTGSGSGTDFTLTISSVEVEDLAVYYC |
| | | HQYLSSWTFGGGTKLEIK |
| 73 | anti-RIP1 HCVR (HVRs | DVQLVESGGGLVQPGGSRKLSCAASGFTFGSFGMHWVRQTPEKRLEW |
| | underlined) | IAYISSGGSTIYYADTVKGRFTISRDTPKNTLFLQMTSLRSEDTAMY |
| | | FCARSIMISTEFDYWGQGTTLTVSS |
| 74 | anti-RIP1 LC1 HVR-L1 | KSSQNVIYTSNQRNYLA |
| 75 | anti-RIP1 LC1 HVR-L2 | WASTRVS |
| 76 | anti-RIP1 LC1 HVR-L3 | HQYLSSWT |
| 74 | anti-RIP1 LC2 HVR-L1 | KSSQNVIYTSNQRNYLA |
| 77 | anti-RIP1 LC2 HVR-L2 | GASNRYT |
| 76 | anti-RIP1 LC2 HVR-L3 | HQYLSSWT |
| 78 | anti-RIP1 HVR-H1 | GFTFGSFGMH |
| 79 | anti-RIP1 HVR-H2 | YISSGGSTIYYADTVKG |
| 80 | anti-RIP1 HVR-H3 | ARSIMISTEFDY |
| 81 | anti-RIP2 LC (with signal | MGWSCIILFLVATATGVHSNIMMTQSPSSLAVSAGEKVTMNCKSSQS |
| | sequence underlined) | VLYSSNQRNYLAWYQQKPGQSPKLLIYWASIRESGVPDRFTGSGSGT |
| | | DFTLTISSVQAEDLAVYYCHQYLSSYTFGGGTKLEIKRADAAPTVSI |
| | | FPPSSEQLTSGGASVVCFLNNFYPKDINVKWKIDGSERQNGVLNSWT |
| | | DQDSKDSTYSMSSTLTLTKDEYERHNSYTCEATHKTSTSPIVKSFNR |
| | | NEC |
| 82 | anti-RIP2 mature LC | NIMMTQSPSSLAVSAGEKVTMNCKSSQSVLYSSNQRNYLAWYQQKPG |
| | (from de novo | QSPKLLIYWASIRESGVPDRFTGSGSGTDFTLTISSVQAEDLAVYYC |
| | sequencing) | HQYLSSYTFGGGTKLEIKRADAAPTVSIFPPSSEQLTSGGASVVCFL |
| | | NNFYPKDINVKWKIDGSERQNGVLNSWTDQDSKDSTYSMSSTLTLTK |
| | | DEYERHNSYTCEATHKTSTSPIVKSFNRNEC |
| 83 | anti-RIP2 LC (with signal | MGWSCIILFLVATATGVHSNIMMTQSPSSLAVSAGEKVTMNCKSSQS |
| | sequence underlined) | VLYSSNQRNYLAWYQQKPGQSPKLLIYWASIRESGVPDRFTGSGSGT |
| | having one amino acid | DFTLTISSVQAEDLAVYYCHQYLSSYTFGGGTKVEIKRTVAAPSVFI |
| | difference L104V | FPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNR |
| | | GEC |
| 84 | anti-RIP2 mature LC | NIMMTQSPSSLAVSAGEKVTMNCKSSQSVLYSSNQRNYLAWYQQKPG |
| | having one amino acid | QSPKLLIYWASIRESGVPDRFTGSGSGTDFTLTISSVQAEDLAVYYC |
| | difference L104V | HQYLSSYTFGGGTKVEIKRTVAAPSVFIFPPSDEQLKSGTASVVCLL |
| | | NNFYPREAKVQWKVDNALQSGNSQESVTEQDSKDSTYSLSSTLTLSK |
| | | ADYEKHKVYACEVTHQGLSSPVTKSFNRGEC |
| 85 | anti-RIP2 HC (with signal | MGWSCIILFLVATATGAYAEVQLQQSGPELEKPGASVKISCKASGYS |
| | sequence underlined) | FTAYNMNWVKQSNGKSLEWIGNIDPFYGDLNYSQKFKGKATLTVDKS |
| | | SSTAYMQLMSLTSEDSAVYYCVRSNYYASVYGAWFAHWGQGTLVTVS |
| | | AAKTTPPSVYPLAPGSAAQTNSMVTLGCLVKGYFPEPVTVTWNSGSL |
| | | SSGVHTFPAVLQSDLYTLSSSVTVPSSTWPSETVTCNVAHPASSTKV |
| | | DKKIVPRDCGCKPCICTVPEVSSVFIFPPKPKDVLTITLTPKVTCVV |
| | | VDISKDDPEVQFSWFVDDVEVHTAQTQPREEQFNSTERSVSELPIMH |
| | | QDWLNGKEFKCRVNSAAFPAPIEKTISKTKGRPKAPQVYTIPPPKEQ |
| | | MAKDKVSLTCMITDFFPEDITVEWQWNGQPAENYKNTQPIMDTDGSY |
| | | FVYSKLNVQKSNWEAGNTFTCSVLHEGLHNHHTEKSLSHSPGK |
| 86 | anti-RIP2 mature HC | EVQLQQSGPELEKPGASVKISCKASGYSFTAYNMNWVKQSNGKSLEW |
| | (from de novo | IGNIDPFYGDLNYSQKFKGKATLTVDKSSSTAYMQLMSLTSEDSAVY |
| | sequencing) | YCVRSNYYASVYGAWFAHWGQGTLVTVSAAKTTPPSVYPLAPGSAAQ |
| | | TNSMVTLGCLVKGYFPEPVTVTWNSGSLSSGVHTFPAVLQSDLYTLS |
| | | SSVTVPSSTWPSETVTCNVAHPASSTKVDKKIVPRDCGCKPCICTVP |
| | | EVSSVFIFPPKPKDVLTITLTPKVTCVVVDISKDDPEVQFSWFVDDV |
| | | EVHTAQTQPREEQFNSTFRSVSELPIMHQDWLNGKEFKCRVNSAAFP |
| | | APIEKTISKTKGRPKAPQVYTIPPPKEQMAKDKVSLTCMITDFFPED |
| | | ITVEWQWNGQPAENYKNTQPIMDTDGSYFVYSKLNVQKSNWEAGNTF |
| | | TCSVLHEGLHNHHTEKSLSHSPGK |
| 87 | anti-RIP2 HC (with signal | MGWSCIILFLVATATGAYAEVQLQQSGPELEKPGASVKISCKASGYS |
| | sequence underlined) | FTAYNMNWVKQSNGKSLEWIGNIDPFYGDLNYSQKFKGKATLTVDKS |
| | having one amino acid | SSTAYMQLMSLTSEDSAVYYCVRSNYYASVYGAWFAHWGQGTLVTVS |
| | difference A113S | SAKTTPPSVYPLAPGSAAQTNSMVTLGCLVKGYFPEPVTVTWNSGSL |
| | | SSGVHTFPAVLQSDLYTLSSSVTVPSSTWPSETVTCNVAHPASSTKV |
| | | DKKIVPRDCGCKPCICTVPEVSSVFIFPPKPKDVLTITLTPKVTCVV |
| | | VDISKDDPEVQFSWFVDDVEVHTAQTQPREEQFNSTFRSVSELPIMH |
| | | QDWLNGKEFKCRVNSAAFPAPIEKTISKTKGRPKAPQVYTIPPPKEQ |
| | | MAKDKVSLTCMITDFFPEDITVEWQWNGQPAENYKNTQPIMDTDGSY |
| | | FVYSKLNVQKSNWEAGNTFTCSVLHEGLHNHHTEKSLSHSPGK |
| 88 | anti-RIP2 mature HC | EVQLQQSGPELEKPGASVKISCKASGYSFTAYNMNWVKQSNGKSLEW |
| | having one amino acid | IGNIDPFYGDLNYSQKFKGKATLTVDKSSSTAYMQLMSLTSEDSAVY |
| | difference A113S | YCVRSNYYASVYGAWFAHWGQGTLVTVSSAKTTPPSVYPLAPGSAAQ |
| | | TNSMVTLGCLVKGYFPEPVTVTWNSGSLSSGVHTFPAVLQSDLYTLS |
| | | SSVTVPSSTWPSETVTCNVAHPASSTKVDKKIVPRDCGCKPCICTVP |
| | | EVSSVFIFPPKPKDVLTITLTPKVTCVVVDISKDDPEVQFSWFVDDV |
| | | EVHTAQTQPREEQFNSTFRSVSELPIMHQDWLNGKEFKCRVNSAAFP |
| | | APIEKTISKTKGRPKAPQVYTIPPPKEQMAKDKVSLTCMITDFFPED |
| | | ITVEWQWNGQPAENYKNTQPIMDTDGSYFVYSKLNVQKSNWEAGNTF |
| | | TCSVLHEGLHNHHTEKSLSHSPGK |
| 89 | anti-RIP2 knob HC | MGWSCIILFLVATATGAYAEVQLQQSGPELEKPGASVKISCKASGYS |
| | having one amino acid | FTAYNMNWVKQSNGKSLEWIGNIDPFYGDLNYSQKFKGKATLTVDKS |
| | difference A113S (with | SSTAYMQLMSLTSEDSAVYYCVRSNYYASVYGAWFAHWGQGTLVTVS |
| | signal sequence | SASTKGPSVFPLAPSSKSTSGGTAALGCLVKDYFPEPVTVSWNSGAL |
| | underlined) | TSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTL |
| | | PPSREEMTKNQVSLWCLVKGFYPSDIAVEWESNGQPENNYKTTPPVL |
| | | DSDGSFFLYSKLTVDKSRWQQGNVFSCSVMHEALHNHYTQKSLSLSP |
| | | GK |
| 90 | anti-RIP2 mature knob | EVQLQQSGPELEKPGASVKISCKASGYSFTAYNMNWVKQSNGKSLEW |
| | HC having one amino acid | IGNIDPFYGDLNYSQKFKGKATLTVDKSSSTAYMQLMSLTSEDSAVY |
| | difference A113S | YCVRSNYYASVYGAWFAHWGQGTLVTVSSASTKGPSVFPLAPSSKST |
| | | SGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKV |
| | | SNKALPAPIEKTISKAKGQPREPQVYTLPPSREEMTKNQVSLWCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 91 | anti-RIP2 hole HC having | MGWSCIILFLVATATGAYAEVQLQQSGPELEKPGASVKISCKASGYS |
| | one amino acid difference | FTAYNMNWVKQSNGKSLEWIGNIDPFYGDLNYSQKFKGKATLTVDKS |
| | A113S (with signal | SSTAYMQLMSLTSEDSAVYYCVRSNYYASVYGAWFAHWGQGTLVTVS |
| | sequence underlined) | SASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTL |
| | | PPSREEMTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| | | GK |
| 92 | anti-RIP2 mature hole HC | EVQLQQSGPELEKPGASVKISCKASGYSFTAYNMNWVKQSNGKSLEW |
| | having one amino acid | IGNIDPFYGDLNYSQKFKGKATLTVDKSSSTAYMQLMSLTSEDSAVY |
| | difference A113S | YCVRSNYYASVYGAWFAHWGQGTLVTVSSASTKGPSVFPLAPSSKST |
| | | SGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKV |
| | | SNKALPAPIEKTISKAKGQPREPQVYTLPPSREEMTKNQVSLSCAVK |
| | | GFYPSDIAVEWESNGQPENNYKTTPPVLDSDGSFFLVSKLTVDKSRW |
| | | QQGNVFSCSVMHEALHNHYTQKSLSLSPGK |
| 93 | anti-RIP2 LCVR (HVRs | NIMMTQSPSSLAVSAGEKVTMNCKSSQSVLYSSNQRNYLAWYQQKPG |
| | underlined) having one | QSPKLLIYWASIRESGVPDRFTGSGSGTDFTLTISSVQAEDLAVYYC |
| | amino acid difference | HQYLSSYTFGGGTKVEIK |
| | L104V | |
| 94 | anti-RIP2 HCVR (HVRs | EVQLQQSGPELEKPGASVKISCKASGYSFTAYNMNWVKQSNGKSLEW |
| | underlined) having one | IGNIDPFYGDLNYSQKFKGKATLTVDKSSSTAYMQLMSLTSEDSAVY |
| | amino acid difference | YCVRSNYYASVYGAWFAHWGQGTLVTVSS |
| | A113S | |
| 95 | anti-RIP2 HVR-L1 | KSSQSVLYSSNQRNYLA |
| 96 | anti-RIP2 HVR-L2 | WASIRES |
| 97 | anti-RIP2 HVR-L3 | HQYLSSYT |
| 98 | anti-RIP2 HVR-H1 | GYSFTAYNMN |
| 99 | anti-RIP2 HVR-H2 | NIDPFYGDLNYSQKFKG |
| 100 | anti-RIP2 HVR-H3 | VRSNYYASVYGAWFAH |
| 101 | RIP2 polypeptide | MNGEAICSALPTIPYHKLADLRYLSRGASGTVSSARHADWRVQVAVK |
| | sequence (UniProt | HLHIHTPLLDSERKDVLREAEILHKARFSYILPILGICNEPEFLGIV |
| | 043353) | TEYMPNGSLNELLHRKTEYPDVAWPLRFRILHEIALGVNYLHNMTPP |
| | | LLHHDLKTQNILLDNEFHVKIADFGLSKWRMMSLSQSRSSKSAPEGG |
| | | TIIYMPPENYEPGQKSRASIKHDIYSYAVITWEVLSRKQPFEDVTNP |
| | | LQIMYSVSQGHRPVINEESLPYDIPHRARMISLIESGWAQNPDERPS |
| | | FLKCLIELEPVLRTFEEITFLEAVIQLKKTKLQSVSSAIHLCDKKKM |
| | | ELSLNIPVNHGPQEESCGSSQLHENSGSPETSRSLPAPQDNDFLSRK |
| | | AQDCYFMKLHHCPGNHSWDSTISGSQRAAFCDHKTTPCSSAIINPLS |
| | | TAGNSERLQPGIAQQWIQSKREDIVNQMTEACLNQSLDALLSRDLIM |
| | | KEDYELVSTKPTRTSKVRQLLDTTDIQGEEFAKVIVQKLKDNKQMGL |
| | | QPYPEILVVSRSPSLNLLQNKSM |
| 102 | C-terminal residues 529- | SPSLNLLQNKSM |
| | 540 of RIP2 polypeptide | |
| 103 | RIP1 polypeptide | MQPDMSLNVIKMKSSDFLESAELDSGGFGKVSLCFHRTQGLMIMKTV |
| | (UniProt Q13546) | YKGPNCIEHNEALLEEAKMMNRLRHSRVVKLLGVIIEEGKYSLVMEY |
| | | MEKGNLMHVLKAEMSTPLSVKGRIILEIIEGMCYLHGKGVIHKDLKP |
| | | ENILVDNDFHIKIADLGLASFKMWSKLNNEEHNELREVDGTAKKNGG |
| | | TLYYMAPEHLNDVNAKPTEKSDVYSFAVVLWAIFANKEPYENAICEQ |
| | | QLIMCIKSGNRPDVDDITEYCPREIISLMKLCWEANPEARPTFPGIE |
| | | EKFRPFYLSQLEESVEEDVKSLKKEYSNENAVVKRMQSLQLDCVAVP |
| | | SSRSNSATEQPGSLHSSQGLGMGPVEESWFAPSLEHPQEENEPSLQS |
| | | KLQDEANYHLYGSRMDRQTKQQPRQNVAYNREEERRRRVSHDPFAQQ |
| | | RPYENFQNTEGKGTAYSSAASHGNAVHQPSGLTSQPQVLYQNNGLYS |
| | | SHGFGTRPLDPGTAGPRVWYRPIPSHMPSLHNIPVPETNYLGNTPTM |
| | | PFSSLPPTDESIKYTIYNSTGIQIGAYNYMEIGGTSSSLLDSTNTNF |
| | | KEEPAAKYQAIFDNTTSLTDKHLDPIRENLGKHWKNCARKLGFTQSQ |
| | | IDEIDHDYERDGLKEKVYQMLQKWVMREGIKGATVGKLAQALHQCSR |
| | | IDLLSSLIYVSQN |
| 104 | TRADD: 125-134 | LDALLADEER |
| 105 | TNFR1: 415-423 | EATLELLGR |
| 106 | RIP1: 154-163 | IADLGLASFK |
| 107 | NQD2: 107-118 | LIAAAQEAQADSQSPK |
| 108 | IRAK1: 343-351 | SSNVLLDER |

HC = heavy chain; LC = light chain; HCVR = heavy chain variable region; LCVR = light chain variable region; HVR = hypervariable region.

1. A method of determining the presence of a polyubiquitinated protein in a sample suspected of containing a polyubiquitinated protein, wherein the polyubiquitinated protein is a pro-inflammatory protein and comprises a polyubiquitin,
comprising exposing the sample to at least one multispecific antibody comprising a first half antibody comprising a first antigen binding site that binds to a polyubiquitin; and a second half antibody comprising a second antigen binding site that binds the pro-inflammatory protein, and
determining the binding of the at least one antibody to a polyubiquitinated protein in the sample.

2. The method of claim 1, wherein the polyubiquitinated protein comprises a M1-linked polyubiquitin and/or a K63-linked polyubiquitin.

3. The method of claim 1, wherein the pro-inflammatory protein is a component of one or more signaling complexes, or wherein the pro-inflammatory protein has an elevated level of ubiquitination in the inflammatory state relative to the level of ubiquitination when not in the inflammatory state.

4. The method of claim 1, wherein the pro-inflammatory protein is receptor-interacting protein kinase 1 (RIP1), receptor-interacting protein kinase 2 (RIP2), cellular inhibitors of apoptosis 1 and 2 (c-IAP1/2), Tumor Necrosis Factor Receptor 1 (TNFR1), linear ubiquitin chain assembly complex (LUBAC), and/or nuclear factor-kappa B (NF-κB) essential modulator (NEMO).

5-7. (canceled)

8. The method of claim 1, wherein the pro-inflammatory protein has a level of ubiquitination of at least 1-fold, 2-fold, 3-fold, 4-fold, 5-fold, 6-fold, 7-fold, 8-fold, 9-fold, 10-fold, 11-fold, 12-fold, 1-fold to 12-fold, 2-fold to 12-fold, 3-fold to 12-fold, 4-fold to 12-fold, 5-fold to 12-fold, 6-fold to 12-fold, 7-fold to 12-fold, 8-fold to 12-fold, 9-fold to 12-fold, 10-fold to 12-fold, or 11-fold to 12-fold in the inflammatory state relative to the level of ubiquitination when not in the inflammatory state.

9. The method of claim 3, wherein an elevated level of ubiquitination correlates to an increase in severity of an inflammatory disease state.

10. The method of claim 9, wherein the pro-inflammatory protein is associated with an inflammatory disease, such as inflammatory bowel disease, Crohn's disease, diverticulitis, and ulcerative colitis.

11-12. (canceled)

13. A multispecific antibody comprising a first half antibody comprising a first antigen binding site that binds to a polyubiquitin; and a second half antibody comprising a second antigen binding site that binds receptor-interacting protein kinase 1 (RIP1) or receptor-interacting protein kinase 2 (RIP2).

14. The multispecific antibody of claim 13, which selectively recognizes polyubiquitinated RIP1.

15. The multispecific antibody of claim 13, which does not recognize receptor-interacting protein kinase 2 (RIP2) and/or does not recognize non-ubiquitinated RIP1.

16. (canceled)

17. The multispecific antibody of claim 13, which selectively recognizes polyubiquitinated RIP2.

18. The multispecific antibody of claim 17, wherein the multispecific antibody does not recognize RIP1, XIAP, and/or c-IAP1, and/or does not recognize non-ubiquitinated RIP2.

19. (canceled)

20. The multispecific antibody of claim 13, wherein the polyubiquitin comprises a K11 linkage, a K48 linkage, a K63 linkage, or a M1 linkage.

21-23. (canceled)

24. The multispecific antibody of claim 13, comprising a first half antibody that comprises:
a) (i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14; or
b) (i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28; or
c) (i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42; or
d) (i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56.

25. The multispecific antibody of claim 13, comprising a second half antibody that comprises:
a) (i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
b) (i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
(c) (i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100.

26. (canceled)

27. The multispecific antibody of claim 13, comprising first and second half antibodies, wherein
a) the first half antibody comprises
(i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14,
and the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
b) the first half antibody comprises
(i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14,
and the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
c) the first half antibody comprises
(i) HVR-L1 comprising the amino acid sequence of SEQ ID NO: 9,
(ii) HVR-L2 comprising the amino acid sequence of SEQ ID NO: 10,
(iii) HVR-L3 comprising the amino acid sequence of SEQ ID NO: 11,
(iv) HVR-H1 comprising the amino acid sequence of SEQ ID NO: 12,
(v) HVR-H2 comprising the amino acid sequence of SEQ ID NO: 13, and
(vi) HVR-H3 comprising the amino acid sequence of SEQ ID NO: 14,
and the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100; or
d) the first half antibody comprises
(i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28,
and the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
e) the first half antibody comprises
(i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28,
and the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
f) the first half antibody comprises
(i) HVR-L1 comprising the amino acid sequence of SEQ ID NO: 23,
(ii) HVR-L2 comprising the amino acid sequence of SEQ ID NO: 24,
(iii) HVR-L3 comprising the amino acid sequence of SEQ ID NO: 25,
(iv) HVR-H1 comprising the amino acid sequence of SEQ ID NO: 26,
(v) HVR-H2 comprising the amino acid sequence of SEQ ID NO: 27, and
(vi) HVR-H3 comprising the amino acid sequence of SEQ ID NO: 28,
and the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100; or
g) the first half antibody comprises
(i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42,
and the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
h) the first half antibody comprises
(i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42,
and the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
i) the first half antibodies comprises
(i) HVR-L1 comprising the amino acid sequence of SEQ ID NO: 37,
(ii) HVR-L2 comprising the amino acid sequence of SEQ ID NO: 38,
(iii) HVR-L3 comprising the amino acid sequence of SEQ ID NO: 39,
(iv) HVR-H1 comprising the amino acid sequence of SEQ ID NO: 40,
(v) HVR-H2 comprising the amino acid sequence of SEQ ID NO: 41, and
(vi) HVR-H3 comprising the amino acid sequence of SEQ ID NO: 42,
and the second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100; or
j) the first half antibody comprises
(i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56,
and the second half antibody comprises
(i) HVR-L1 comprising the amino acid sequence of SEQ ID NO: 74,
(ii) HVR-L2 comprising the amino acid sequence of SEQ ID NO: 75,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
k) the first half antibody comprises
(i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56,
and the second half antibody comprises
(i) HVR-L1 comprising the amino acid sequence of SEQ ID NO: 74,
(ii) HVR-L2 comprising the amino acid sequence of SEQ ID NO: 77,
(iii) HVR-L3 comprising the amino acid sequence of SEQ ID NO: 76,
(iv) HVR-H1 comprising the amino acid sequence of SEQ ID NO: 78,
(v) HVR-H2 comprising the amino acid sequence of SEQ ID NO: 79, and
(vi) HVR-H3 comprising the amino acid sequence of SEQ ID NO: 80; or
1) the first half antibody comprises
(i) HVR-L1 comprising the amino acid sequence of SEQ ID NO: 51,
(ii) HVR-L2 comprising the amino acid sequence of SEQ ID NO: 52,
(iii) HVR-L3 comprising the amino acid sequence of SEQ ID NO: 53,
(iv) HVR-H1 comprising the amino acid sequence of SEQ ID NO: 54,
(v) HVR-H2 comprising the amino acid sequence of SEQ ID NO: 55, and
(vi) HVR-H3 comprising the amino acid sequence of SEQ ID NO: 56,
and the other second half antibody comprises
(i) HVR-L1 comprising the amino acid sequence of SEQ ID NO: 95,
(ii) HVR-L2 comprising the amino acid sequence of SEQ ID NO: 96,
(iii) HVR-L3 comprising the amino acid sequence of SEQ ID NO: 97,
(iv) HVR-H1 comprising the amino acid sequence of SEQ ID NO: 98,
(v) HVR-H2 comprising the amino acid sequence of SEQ ID NO: 99, and
(vi) HVR-H3 comprising the amino acid sequence of SEQ ID NO: 100.

28-30. (canceled)

31. The multispecific antibody of claim 13, wherein:
a) one of the first and second half antibodies comprises a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8,
and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73; or
b) one of the first and second half antibodies comprises a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8,
and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73; or
c) one of the first and second half antibodies comprises a VL sequence with at least about 95%, such as 96%, 97%, 98%, 99%, or 100% sequence identity to SEQ ID NO: 7 and a VH sequence with at least about 95%, such as 96%, 97%, 98%, 99%, or 100% sequence identity to SEQ ID NO: 8,
and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94; or
d) one of the first and second half antibodies comprises a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22,
and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73; or
e) one of the first and second half antibodies comprises a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22,
and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73; or
f) one of the first and second half antibodies comprises a VL sequence with at least about 95%, such as 96%, 97%, 98%, 99%, or 100% sequence identity to SEQ ID NO: 21 and a VH sequence with at least about 95%, such as 96%, 97%, 98%, 99%, or 100% sequence identity to SEQ ID NO: 22,
and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94; or
g) one of the first and second half antibodies comprises a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36,
and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73; or
h) one of the first and second half antibodies comprises a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36,
and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73; or
i) one of the first and second half antibodies comprises a VL sequence with at least about 95%, such as 96%, 97%, 98%, 99%, or 100% sequence identity to SEQ ID NO: 35 and a VH sequence with at least about 95%, such as 96%, 97%, 98%, 99%, or 100% sequence identity to SEQ ID NO: 36,
and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94; or
j) one of the first and second half antibodies comprises a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50,
and the other of the first and second half antibodies comprises a VL sequence with at least about 95%, such as 96%, 97%, 98%, 99%, or 100% sequence identity to SEQ ID NO: 71 and a VH sequence with at least about 95%, such as 96%, 97%, 98%, 99%, or 100% sequence identity to SEQ ID NO: 73; or
k) one of the first and second half antibodies comprises a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50,
and the other of the first and second half antibodies comprises a VL sequence with at least about 95%, such as 96%, 97%, 98%, 99%, or 100% sequence identity to SEQ ID NO: 72 and a VH sequence with at least about 95%, such as 96%, 97%, 98%, 99%, or 100% sequence identity to SEQ ID NO: 73; or
l) one of the first and second half antibodies comprises a VL sequence with at least about 95%, such as 96%, 97%, 98%, 99%, or 100% sequence identity to SEQ ID NO: 49 and a VH sequence with at least about 95%, such as 96%, 97%, 98%, 99%, or 100% sequence identity to SEQ ID NO: 50,
and the other of the first and second half antibodies comprises a VL sequence with at least about 95%, such as 96%, 97%, 98%, 99%, or 100% sequence identity to SEQ ID NO: 93 and a VH sequence with at least about 95%, such as 96%, 97%, 98%, 99%, or 100% sequence identity to SEQ ID NO: 94.

32-34. (canceled)

35. The multispecific antibody of claim 13, wherein:
a) one of the first and second half antibodies comprises a VL sequence of SEQ ID NO: 7 and a VH sequence of SEQ ID NO: 8,
and the other of the first and second half antibodies comprises a VL sequence of SEQ ID NO: 71 and a VH sequence of SEQ ID NO: 73; or
b) one of the first and second half antibodies comprises a VL sequence of SEQ ID NO: 7 and a VH sequence of SEQ ID NO: 8,
and the other of the first and second half antibodies comprises a VL sequence of SEQ ID NO: 72 and a VH sequence of SEQ ID NO: 73; or
c) one of the first and second half antibodies comprises a VL sequence of SEQ ID NO: 7 and a VH sequence of SEQ ID NO: 8,
and the other of the first and second half antibodies comprises a VL sequence of SEQ ID NO: 93 and a VH sequence of SEQ ID NO: 94; or
d) one of the first and second half antibodies comprises a VL sequence of SEQ ID NO: 21 and a VH sequence of SEQ ID NO: 22,
and the other of the first and second half antibodies comprises a VL sequence of SEQ ID NO: 71 and a VH sequence of SEQ ID NO: 73; or
e) one of the first and second half antibodies comprises a VL sequence of SEQ ID NO: 21 and a VH sequence of SEQ ID NO: 22,
and the other of the first and second half antibodies comprises a VL sequence of SEQ ID NO: 72 and a VH sequence of SEQ ID NO: 73; or
f) one of the first and second half antibodies comprises a VL sequence of SEQ ID NO: 21 and a VH sequence of SEQ ID NO: 22,
and the other of the first and second half antibodies comprises a VL sequence of SEQ ID NO: 93 and a VH sequence of SEQ ID NO: 94; or
g) one of the first and second half antibodies comprises a VL sequence of SEQ ID NO: 35 and a VH sequence of SEQ ID NO: 36,
and the other of the first and second half antibodies comprises a VL sequence of SEQ ID NO: 71 and a VH sequence of SEQ ID NO: 73; or
h) one of the first and second half antibodies comprises a VL sequence of SEQ ID NO: 35 and a VH sequence of SEQ ID NO: 36,
and the other of the first and second half antibodies comprises a VL sequence of SEQ ID NO: 72 and a VH sequence of SEQ ID NO: 73; or
i) one of the first and second half antibodies comprises a VL sequence of SEQ ID NO: 35 and a VH sequence of SEQ ID NO: 36,
and the other of the first and second half antibodies comprises a VL sequence of SEQ ID NO: 93 and a VH sequence of SEQ ID NO: 94; or
j) one of the first and second half antibodies comprises a VL sequence of SEQ ID NO: 49 and a VH sequence of SEQ ID NO: 50,
and the other of the first and second half antibodies comprises a VL sequence of SEQ ID NO: 71 and a VH sequence of SEQ ID NO: 73; or
k) one of the first and second half antibodies comprises a VL sequence of SEQ ID NO: 49 and a VH sequence of SEQ ID NO: 50,
and the other of the first and second half antibodies comprises a VL sequence of SEQ ID NO: 72 and a VH sequence of SEQ ID NO: 73; or
l) one of the first and second half antibodies comprises a VL sequence of SEQ ID NO: 49 and a VH sequence of SEQ ID NO: 50,
and the other of the first and second half antibodies comprises a VL sequence of SEQ ID NO: 93 and a VH sequence of SEQ ID NO: 94.

36-79. (canceled)

80. The multispecific antibody of claim 13, wherein:
a) the first half antibody comprises a light chain sequence of SEQ ID NO: 2 and a heavy chain sequence of SEQ ID NO: 4,
and the second half antibody comprises a light chain sequence of SEQ ID NO: 60 and a heavy chain sequence of SEQ ID NO: 70; or
b) the first half antibody comprises a light chain sequence of SEQ ID NO: 2 and a heavy chain sequence of SEQ ID NO: 6,
and the second half antibody comprises a light chain sequence of SEQ ID NO: 60 and a heavy chain sequence of SEQ ID NO: 68; or
c) the first half antibody comprises a light chain sequence of SEQ ID NO: 16 and a heavy chain sequence of SEQ ID NO: 18,
and the second half antibody comprises a light chain sequence of SEQ ID NO: 60 and a heavy chain sequence of SEQ ID NO: 70; or
d) the first half antibody comprises a light chain sequence of SEQ ID NO: 16 and a heavy chain sequence of SEQ ID NO: 20,
and the second half antibody comprises a light chain sequence of SEQ ID NO: 60 and a heavy chain sequence of SEQ ID NO: 68; or
e) the first half antibody comprises a light chain sequence of SEQ ID NO: 30 and a heavy chain sequence of SEQ ID NO: 32,
and the second half antibody comprises a light chain sequence of SEQ ID NO: 60 and a heavy chain sequence of SEQ ID NO: 70; or
f) the first half antibody comprises a light chain sequence of SEQ ID NO: 30 and a heavy chain sequence of SEQ ID NO: 34,
and the second half antibody comprises a light chain sequence of SEQ ID NO: 60 and a heavy chain sequence of SEQ ID NO: 68; or
g) the first half antibody comprises a light chain sequence of SEQ ID NO: 44 and a heavy chain sequence of SEQ ID NO: 46,
and the second half antibody comprises a light chain sequence of SEQ ID NO: 60 and a heavy chain sequence of SEQ ID NO: 70; or
h) the first half antibody comprises a light chain sequence of SEQ ID NO: 44 and a heavy chain sequence of SEQ ID NO: 48,
and the second half antibody comprises a light chain sequence of SEQ ID NO: 60 and a heavy chain sequence of SEQ ID NO: 68, optionally wherein a C-terminal lysine is missing from one or more heavy chains.

81-103. (canceled)

104. An immunoconjugate comprising the multispecific antibody of claim 13 and a cytotoxic agent or an anti-inflammatory agent.

105. A pharmaceutical formulation comprising a pharmaceutically acceptable carrier and
the immunoconjugate of claim 104;
optionally wherein the pharmaceutical formulation is substantially free of monospecific antibodies, unassembled half antibodies, or both monospecific antibodies and unassembled half antibodies.

106. (canceled)

107. An isolated nucleic acid encoding the multispecific antibody of claim 13.

108. A vector comprising the nucleic acid of claim 107.

109. A host cell comprising the nucleic acid of claim 107.

110. A method of producing an antibody comprising culturing the host cell of claim 109 under conditions wherein the antibody is produced.

111. The method of claim 110, further comprising recovering the antibody from the host cell.

112. A method of making the multispecific antibody of claim 13, comprising forming the antibody from a first half antibody and a second half antibody.

113. (canceled)

114. A method of determining the presence of a polyubiquitinated protein in a sample suspected of containing a polyubiquitin or polyubiquitinated protein, comprising exposing the sample to the multispecific antibody of claim 13 and determining the binding of the antibody to a polyubiquitinated protein in the sample.

115. A method of separating K11-linked polyubiquitinated protein from non-K11-linked polyubiquitinated protein in a sample, or separating K48-linked polyubiquitinated protein from non-K48-linked polyubiquitinated protein in a sample, or separating K63-linked polyubiquitinated protein from non-K63-linked polyubiquitinated protein in a sample, or separating M1-linked polyubiquitinated protein from non-M1-linked polyubiquitinated protein in a sample, comprising contacting the sample with the multispecific antibody of claim 13.

116-118. (canceled)

119. A method of determining the function and/or activity of a polyubiquitinated protein in a cell or sample comprising contacting the cell or sample with the multispecific antibody of claim 13 and assessing the effect of said contacting step on the cell or sample.

120-132. (canceled)

133. A pharmaceutical formulation comprising a pharmaceutically acceptable carrier and the multispecific antibody of claim 13;
optionally wherein the pharmaceutical formulation is substantially free of monospecific antibodies, unassembled half antibodies, or both monospecific antibodies and unassembled half antibodies.
